SEQUENCE LISTING (64 sequences)

SEQ ID NO: 1 - protein, 540 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 532 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 540 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 535 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 532 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 6 - protein, 530 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - protein, 540 aa, synthetic construct
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - protein, 540 aa, synthetic construct
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 9 - protein, 540 aa, synthetic construct
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - DNA, 1623 bp, synthetic construct
  source 1..1623: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggatctggcggtggcggttctggcggtggaggctctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctga

SEQ ID NO: 11 - DNA, 1599 bp, synthetic construct
  source 1..1599: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggcggaggtggaggcggttctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctga

SEQ ID NO: 12 - DNA, 1623 bp, synthetic construct
  source 1..1623: mol_type = other DNA
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggtggcggtggatctggcggtggcggttctggcggtggaggctctatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttga

SEQ ID NO: 13 - DNA, 1608 bp, synthetic construct
  source 1..1608: mol_type = other DNA
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggtggcggtggatctggcggtggcggttctatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttga

SEQ ID NO: 14 - DNA, 1599 bp, synthetic construct
  source 1..1599: mol_type = other DNA
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggcggaggtggaggcggttctatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttga

SEQ ID NO: 15 - DNA, 1593 bp, synthetic construct
  source 1..1593: mol_type = other DNA
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggcggaggtggatctatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttga

SEQ ID NO: 16 - DNA, 1623 bp, synthetic construct
  source 1..1623: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagctgccggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggatctggcggtggcggttctggcggtggaggctctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctga

SEQ ID NO: 17 - DNA, 1623 bp, synthetic construct
  source 1..1623: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagctgccggagatgctggccagtacacctgtcacaaaggaggcgaggttctaagccattcgctcctgctgcttcacgctaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggatctggcggtggcggttctggcggtggaggctctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctga

SEQ ID NO: 18 - DNA, 1623 bp, synthetic construct
  source 1..1623: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagctgccggagatgctggccagtacacctgtcacaaaggaggcgaggttctaagccattcgctcctgctgcttcacgctaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcgcttatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggatctggcggtggcggttctggcggtggaggctctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctga

SEQ ID NO: 19 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 20 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 21 - protein, 219 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 22 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 23 - protein, 15 aa, synthetic construct
GGGGSGGGGSGGGGS

SEQ ID NO: 24 - protein, 10 aa, synthetic construct
GGGGSGGGGS

SEQ ID NO: 25 - protein, 7 aa, synthetic construct
GGGGGGS

SEQ ID NO: 26 - protein, 5 aa, synthetic construct
GGGGS

SEQ ID NO: 27 - DNA, 984 bp, synthetic construct
  source 1..984: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 28 - DNA, 921 bp, synthetic construct
  source 1..921: mol_type = other DNA
atatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttga

SEQ ID NO: 29 - DNA, 657 bp, synthetic construct
  source 1..657: mol_type = other DNA
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 30 - protein, 594 aa, synthetic construct
AGAAACCTCCCCGTGGCCACTCCAGACCCAGGAATGTTCCCATGCCTTCACCACTCCCAAAACCTGCTGAGGGCCGTCAGCAACATGCTCCAGAAGGCCAGACAAACTCTAGAATTTTACCCTTGCACTTCTGAAGAGATTGATCATGAAGATATCACAAAAGATAAAACCAGCACAGTGGAGGCCTGTTTACCATTGGAATTAACCAAGAATGAGAGTTGCCTAAATTCCAGAGAGACCTCTTTCATAACTAATGGGAGTTGCCTGGCCTCCAGAAAGACCTCTTTTATGATGGCCCTGTGCCTTAGTAGTATTTATGAAGACTTGAAGATGTACCAGGTGGAGTTCAAGACCATGAATGCAAAGCTTCTGATGGATCCTAAGAGGCAGATCTTTCTAGATCAAAACATGCTGGCAGTTATTGATGAGCTGATGCAGGCCCTGAATTTCAACAGTGAGACTGTGCCACAAAAATCCTCCCTTGAAGAACCGGATTTTTATAAAACTAAAATCAAGCTCTGCATACTTCTTCATGCTTTCAGAATTCGGGCAGTGACTATTGATAGAGTGATGAGCTATCTGAATGCTTCCTGA

SEQ ID NO: 31 - DNA, 45 bp, synthetic construct
  source 1..45: mol_type = other DNA
ggtggcggtggatctggcggtggcggttctggcggtggaggctct

SEQ ID NO: 32 - DNA, 30 bp, synthetic construct
  source 1..30: mol_type = other DNA
ggtggcggtggatctggcggtggcggttct

SEQ ID NO: 33 - DNA, 21 bp, synthetic construct
  source 1..21: mol_type = other DNA
ggcggaggtggaggcggttct

SEQ ID NO: 34 - DNA, 15 bp, synthetic construct
  source 1..15: mol_type = other DNA
ggcggaggtggatct

SEQ ID NO: 35 - protein, 543 aa, synthetic construct
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHAKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 36 - protein, 543 aa, synthetic construct
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNAYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 37 - protein, 535 aa, synthetic construct
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 38 - protein, 1632 aa, synthetic construct
ATGTGTCCTCAGAAGCTAACCATCTCCTGGTTTGCCATCGTTTTGCTGGTGTCTCCACTCATGGCCATGTGGGAGCTGGAGAAAGACGTTTATGTTGTAGAGGTGGACTGGACTCCCGATGCCCCTGGAGAAACAGTGAACCTCACCTGTGACACGCCTGAAGAAGATGACATCACCTGGACCTCAGACCAGAGACATGGAGTCATAGGCTCTGGAAAGACCCTGACCATCACTGTCAAAGCTGCCCTAGATGCTGGCCAGTACACCTGCCACAAAGGAGGCGAGACTCTGAGCCACTCACATCTGCTGCTCCACAAGAAGGAAAATGGAATTTGGTCCACTGAAATTTTAAAAAATTTCAAAAACAAGACTTTCCTGAAGTGTGAAGCACCAAATTACTCCGGACGGTTCACGTGCTCATGGCTGGTGCAAAGAAACATGGACTTGAAGTTCAACATCAAGAGCAGTAGCAGTTCCCCTGACTCTCGGGCAGTGACATGTGGAATGGCGTCTCTGTCTGCAGAGAAGGTCACACTGGACCAAAGGGACTATGAGAAGTATTCAGTGTCCTGCCAGGAGGATGTCACCTGCCCAACTGCCGAGGAGACCCTGCCCATTGAACTGGCGTTGGAAGCACGGCAGCAGAATGCTTATGAGAACTACAGCACCAGCTTCTTCATCAGGGACATCATCAAACCAGACCCGCCCAAGAACTTGCAGATGAAGCCTTTGAAGAACTCACAGGTGGAGGTCAGCTGGGAGTACCCTGACTCCTGGAGCACTCCCCATTCCTACTTCTCCCTCAAGTTCTTTGTTCGAATCCAGCGCAAGAAAGAAAAGATGAAGGAGACAGAGGAGGGGTGTAACCAGAAAGGTGCGTTCCTCGTAGAGAAGACATCTACCGAAGTCCAATGCAAAGGCGGGAATGTCTGCGTGCAAGCTCAGGATCGCTATTACAATTCCTCATGCAGCAAGTGGGCATGTGTTCCCTGCAGGGTCCGATCCGGTGGCGGTGGATCTGGCGGTGGCGGTTCTGGCGGTGGAGGCTCTAGGGTCATTCCAGTCTCTGGACCTGCCAGGTGTCTTAGCCAGTCCCGAAACCTGCTGAAGACCACAGATGACATGGTGAAGACGGCCAGAGAAAAACTGAAACATTATTCCTGCACTGCTGAAGACATCGATCATGAAGACATCACACGGGACCAAACCAGCACATTGAAGACCTGTTTACCACTGGAACTACACAAGAACGAGAGTTGCCTGGCTACTAGAGAGACTTCTTCCACAACAAGAGGGAGCTGCCTGCCCCCACAGAAGACGTCTTTGATGATGACCCTGTGCCTTGGTAGCATCTATGAGGACTTGAAGATGTACCAGACAGAGTTCCAGGCCATCAACGCAGCACTTCAGAATCACAACCATCAGCAGATCATTCTAGACAAGGGCATGCTGGTGGCCATCGATGAGCTGATGCAGTCTCTGAATCATAATGGCGAGACTCTGCGCCAGAAACCTCCTGTGGGAGAAGCAGACCCTTACAGAGTGAAAATGAAGCTCTGCATCCTGCTTCACGCCTTCAGCACCCGCGTCGTGACCATCAACAGGGTGATGGGCTATCTGAGCTCCGCCTAA

SEQ ID NO: 39 - DNA, 1632 bp, synthetic construct
  source 1..1632: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggatctggcggtggcggttctggcggtggaggctctagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaa

SEQ ID NO: 40 - DNA, 1608 bp, synthetic construct
  source 1..1608: mol_type = other DNA
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggcggaggtggaggcggttctatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctaa

SEQ ID NO: 41 - protein, 335 aa, synthetic construct
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHAKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 42 - protein, 193 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 43 - protein, 335 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 44 - protein, 215 aa, synthetic construct
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 45 - protein, 313 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 46 - DNA, 1005 bp, synthetic construct
  source 1..1005: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagctgccctagatgctggccagtacacctgccacaaaggaggcgagactctgagccactcacatctgctgctccacgccaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcc

SEQ ID NO: 47 - DNA, 582 bp, synthetic construct
  source 1..582: mol_type = other DNA
agggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaa

SEQ ID NO: 48 - DNA, 1005 bp, synthetic construct
  source 1..1005: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcc

SEQ ID NO: 49 - protein, 645 aa, synthetic construct
ATGTGTCAATCACGCTACCTCCTCTTTTTGGCCACCCTTGCCCTCCTAAACCACCTCAGTTTGGCCAGGGTCATTCCAGTCTCTGGACCTGCCAGGTGTCTTAGCCAGTCCCGAAACCTGCTGAAGACCACAGATGACATGGTGAAGACGGCCAGAGAAAAACTGAAACATTATTCCTGCACTGCTGAAGACATCGATCATGAAGACATCACACGGGACCAAACCAGCACATTGAAGACCTGTTTACCACTGGAACTACACAAGAACGAGAGTTGCCTGGCTACTAGAGAGACTTCTTCCACAACAAGAGGGAGCTGCCTGCCCCCACAGAAGACGTCTTTGATGATGACCCTGTGCCTTGGTAGCATCTATGAGGACTTGAAGATGTACCAGACAGAGTTCCAGGCCATCAACGCAGCACTTCAGAATCACAACCATCAGCAGATCATTCTAGACAAGGGCATGCTGGTGGCCATCGATGAGCTGATGCAGTCTCTGAATCATAATGGCGAGACTCTGCGCCAGAAACCTCCTGTGGGAGAAGCAGACCCTTACAGAGTGAAAATGAAGCTCTGCATCCTGCTTCACGCCTTCAGCACCCGCGTCGTGACCATCAACAGGGTGATGGGCTATCTGAGCTCCGCC

SEQ ID NO: 50 - protein, 942 aa, synthetic construct
ATGTGGGAGCTGGAGAAAGACGTTTATGTTGTAGAGGTGGACTGGACTCCCGATGCCCCTGGAGAAACAGTGAACCTCACCTGTGACACGCCTGAAGAAGATGACATCACCTGGACCTCAGACCAGAGACATGGAGTCATAGGCTCTGGAAAGACCCTGACCATCACTGTCAAAGAGTTTCTAGATGCTGGCCAGTACACCTGCCACAAAGGAGGCGAGACTCTGAGCCACTCACATCTGCTGCTCCACAAGAAGGAAAATGGAATTTGGTCCACTGAAATTTTAAAAAATTTCAAAAACAAGACTTTCCTGAAGTGTGAAGCACCAAATTACTCCGGACGGTTCACGTGCTCATGGCTGGTGCAAAGAAACATGGACTTGAAGTTCAACATCAAGAGCAGTAGCAGTTCCCCTGACTCTCGGGCAGTGACATGTGGAATGGCGTCTCTGTCTGCAGAGAAGGTCACACTGGACCAAAGGGACTATGAGAAGTATTCAGTGTCCTGCCAGGAGGATGTCACCTGCCCAACTGCCGAGGAGACCCTGCCCATTGAACTGGCGTTGGAAGCACGGCAGCAGAATAAATATGAGAACTACAGCACCAGCTTCTTCATCAGGGACATCATCAAACCAGACCCGCCCAAGAACTTGCAGATGAAGCCTTTGAAGAACTCACAGGTGGAGGTCAGCTGGGAGTACCCTGACTCCTGGAGCACTCCCCATTCCTACTTCTCCCTCAAGTTCTTTGTTCGAATCCAGCGCAAGAAAGAAAAGATGAAGGAGACAGAGGAGGGGTGTAACCAGAAAGGTGCGTTCCTCGTAGAGAAGACATCTACCGAAGTCCAATGCAAAGGCGGGAATGTCTGCGTGCAAGCTCAGGATCGCTATTACAATTCCTCATGCAGCAAGTGGGCATGTGTTCCCTGCAGGGTCCGATCCTAA

SEQ ID NO: 51 - protein, 19 aa, synthetic construct
MGWSCIILFLVATATGVHS

SEQ ID NO: 52 - protein, 118 aa, synthetic construct
QVQLKESGPVLVAPSQTLSITCTVSGFSLASYNTHWVRQPPGKGLEWLGVIWAGGSTNYNSALMSRLSISKDNSKSQVFLQMNSLQTDDTAMYYCAKRSDDYSWFAYWGQGTLVTVSA

SEQ ID NO: 53 - protein, 108 aa, synthetic construct
ENVLTQSPAIMSASPGEKVTMTCRASSSVSSSYLHWYQQKSGASPKVWIYSTSNLASGVPGRFSGSGSGTSYSLTISSVEAEDAATYYCQQYSGYPITFGAGTKVEVK

SEQ ID NO: 54 - protein, 48 aa, synthetic construct
AKPTTTPAPRPPTPAPTIASQPLSLRPEACRPAAGGAVHTRGLDFACD

SEQ ID NO: 55 - protein, 27 aa, synthetic construct
FWVLVVVGGVLACYSLLVTVAFIIFWV

SEQ ID NO: 56 - protein, 41 aa, synthetic construct
RSKRSRLLHSDYMNMTPRRPGPTRKHYQPYAPPRDFAAYRS

SEQ ID NO: 57 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 57 bp, synthetic construct
  source 1..57: mol_type = other DNA
atgggctggagctgcatcatcctgttcctggtggccaccgccaccggcgtgcacagc

SEQ ID NO: 59 - DNA, 354 bp, synthetic construct
  source 1..354: mol_type = other DNA
caggttcagcttaaggaaagcgggcctgtccttgtagctccctcccagacactttcaatcacatgcaccgtatcagggttttccctcgctagctacaatacccattgggtacggcaaccacccggcaaaggacttgagtggttgggcgttatttgggctggcggttctacaaattacaatagtgcactgatgtcccgcctcagcataagtaaagacaactcaaaatctcaggtatttttgcagatgaacagtcttcagacagatgacaccgccatgtactattgcgcaaaacgatcagacgattactcttggttcgcttactgggggcagggcacattggttaccgtctctgct

SEQ ID NO: 60 - DNA, 324 bp, synthetic construct
  source 1..324: mol_type = other DNA
gaaaatgtcttgactcaatcccctgccataatgagtgcaagtccaggagaaaaggtcactatgacctgccgggcttcaagttcagttagttcaagttatttgcactggtatcaacaaaaaagtggggcctcacccaaggtatggatatacagcacatccaaccttgcttctggtgtgccaggtaggttttctggttccggtagcgggacctcttactccttgaccattagctccgtagaggcagaggacgctgctacctactactgtcaacagtactctggttatcccataacatttggagccggtactaaagttgaagtcaag

SEQ ID NO: 61 - DNA, 144 bp, synthetic construct
  source 1..144: mol_type = other DNA
gcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 62 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 63 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 64 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggcctcagtacagccaccaaggacacctacgacgcccttcacatgcaggccctgccccctcgc

(19) United States
(12) Patent Application Publication
(10) Pub. No.: US 2026/0327284 A1
(43) Pub. Date: Oct. 22, 2026

(54) CAR T-CELLS SECRETING SPECIFICITY DETUNED CYTOKINE IL-12

(71) Applicant: Chimeris UK Ltd., Sawston, Cambridge (GB)

(72) Inventors: Maurizio Mangolini, Sawston, Cambridge (GB); Saket Srivastava, Sawston, Cambridge (GB); Biao Ma, Sawston, Cambridge (GB); Shimobi Onuoha, Sawston, Cambridge (GB); Agnieszka Andres, Sawston, Cambridge (GB)

(21) Appl. No.: 19/483,184

(22) PCT Filed: May 7, 2024
(86) PCT No.: PCT/US2024/028194

§ 371 Date: Nov. 10, 2025

Related U.S. Application Data
(60) Provisional application No. 63/502,001, filed on May 12, 2023.

(51) Int. Cl. C07K 14/54; A61K 35/17; A61K 40/11; A61K 40/31; A61P 35/00; C12N 5/0783

(52) U.S. Cl. CPC C07K 14/5434; A61K 35/17; A61K 40/11; A61K 40/31; A61P 35/00; C12N 5/0636; A61K 2239/15; C12N 2510/00

(57) ABSTRACT

Embodiments of the compositions and methods described herein relate to modified IL-12 protein. The present disclosure also relates to CAR T-cells expressing the modified IL-12 protein, and methods of use of these CAR T-cells as part of T-cell-based immunotherapy.

### RELATED APPLICATIONS AND INCORPORATION BY REFERENCE

This application claims the benefit of U.S. Provisional Ser. No. 63/502,001, filed May 12, 2023, which is hereby incorporated by reference in its entirety.

### REFERENCE TO SEQUENCE LISTING

The present application is being filed along with a Sequence Listing in electronic format. The Sequence Listing is provided as a file entitled CHMRS.003WO.xml created on May 3, 2024 which is 93,736 bytes in size. The information in the electronic format of the Sequence Listing is incorporated herein by reference in its entirety.

### FIELD

The present disclosure relates to methods, cells, and compositions for enhancing the effectiveness of chimeric antigen receptor (CAR) bearing T-cells against solid tumors. In particular, methods, cells, and compositions for CAR T-cells that produce a detuned cytokine IL-12.

### BACKGROUND

Cancer cells express antigens. Despite the presence of such antigens, tumors are generally not readily recognized and eliminated by the host due to sub-optimal activation of immune responses.

Interleukin-12 (IL-12) is a heterodimeric cytokine with multiple biological effects on the immune system. It is composed of two subunits, p35 and p40. IL-12 acts on dendritic cells (DC), leading to increased maturation and antigen presentation, which can allow for the initiation of a T-cell response to tumor specific antigens. It also drives the secretion of IL-12 by DCs, creating a positive feedback mechanism to amplify the response. IL-12 is also a strong pro-inflammatory cytokine that leads to the secretion of other cytokines including tumor necrosis factor-alpha (TNF-α) which, combined with IFN-γ, is a prerequisite for the development of CD4+ cytotoxic T lymphocytes (CTL). Further, IL-12 can promote the activation of innate immune cells such as macrophages and eosinophils through its induction of IFN-γ and other cytokines.

The therapeutic potential of IL-12 has been demonstrated through the robust antitumor activity observed in preclinical studies and potent immune-stimulating potential in humans. However systemic administrations of IL-12 have been shown to be exceedingly toxic and methods to obtain the immunomodulatory effects while limiting toxicity are sought.

### SUMMARY

Disclosed herein is a modified IL-12 protein construct. In some embodiments, the modified IL-12 protein construct includes a p35 subdomain, a linker, and a p40 subdomain. In some embodiments, the linker is 4, 5, 6, 7, 8, 9, 10, 11, 12, 13, 14, or 15 amino acids in length. In some embodiments, the p35 subdomain is on the C terminus, and the p40 subdomain is on the N terminus. In some embodiments, the p35 subdomain is on the N terminus, and the p40 subdomain is on the C terminus. In some embodiments, the linker includes a sequence with at least 80% identity to any one of the sequences of SEQ ID NOS: 23-26. In some embodiments, the p40 subdomain includes a sequence with at least 80% identity to any one of the sequences of SEQ ID NOs: 19 or 20. In some embodiments, the p40 subdomain further includes one, two, three, or four mutations. In some embodiments, the mutations include mutations of E81A, F82A, K106A, or K219A. In some embodiments, the p35 subdomain includes a sequence with at least 80% identity to any one of the sequences of SEQ ID NOs: 21 or 22.

Also disclosed herein is a modified IL-12 protein construct. In some embodiments, the construct includes a sequence with at least 80% identity to any one of the sequences of SEQ ID NOs: 1-9. In some embodiments, a p40 subdomain of the IL-12 protein construct further includes one, two, three, or four mutations. In some embodiments, the mutations include mutations of E81A, F82A, K106A, or K219A.

Also disclosed herein is a nucleotide sequence encoding any one of the modified IL-12 protein constructs of the present disclosure.

Also disclosed herein is a nucleotide sequence expressing a modified IL-12 protein construct. In some embodiments, the nucleotide sequence includes a sequence with at least 80% identity to any one of the sequences of SEQ ID NOs: 10-18.

Also disclosed herein is a vector encoding any one of the modified IL-12 protein constructs of the present disclosure, and/or any one of the nucleotide sequences of the present disclosure.

Also disclosed herein is a cell containing and/or secreting any one of the modified IL-12 protein constructs of the present disclosure, any one of the nucleotide sequences of the present disclosure, and/or any one of the vectors of the present disclosure. In some embodiments, the cell expresses the modified IL-12 protein. In some embodiments, the cell is a lymphocyte and/or leukocyte. In some embodiments, the cell is a T lymphocyte or a cytotoxic T lymphocyte. In some embodiments, the modified IL-12 protein has a reduced affinity for IL-12 receptors compared to wild type IL-12. In some embodiments, the modified IL-12 protein has a reduced stability and/or half-life compared to wild type IL-12. In some embodiments, the modified IL-12 protein has a reduced expression compared to wild type IL-12. In some embodiments, the reduced expression includes reduced concentration of IL-12 in a cell, a tissue, an organ, a blood sample, a plasma sample, a system, and/or a subject. In some embodiments, the cell leads to decreased level of IFNγ production by effector cells when compared to a cell expressing wild type IL-12.

Also disclosed herein is a method of treating cancer in a subject. In some embodiments, the method includes administering to the subject the cell of any one of the embodiments of the present disclosure. In some embodiments, the subject is mammalian and/or human. In some embodiments, the cancer is pancreatic cancer, prostate cancer, breast cancer, ovarian cancer, colorectal cancer, lung cancer, renal cell carcinoma, bladder cancer, medulloblastoma or neuroblastoma.

Also disclosed herein is a method of treating cancer in a subject, the method including administering to the subject a modified IL-12 protein construct including a p35 subdomain, a linker, and a p40 subdomain. In some embodiments, the linker is 4, 5, 6, 7, 8, 9, 10, 11, 12, 13, 14, or 15 amino acids in length. In some embodiments, the p35 subdomain is on the C terminus, and the p40 subdomain is on the N terminus. In some embodiments, the p35 subdomain is on the N terminus, and the p40 subdomain is on the C terminus. In some embodiments, the linker includes a sequence with at least 80% identity to any one of the sequences of SEQ ID NOs: 23-26. In some embodiments, the p40 subdomain includes a sequence with at least 80% identity to any one of the sequences of SEQ ID NOs: 19 or 20. In some embodiments, the p40 subdomain further includes one, two, three, or four mutations. In some embodiments, the mutations include mutations of E81A, F82A, K106A, or K219A. In some embodiments, the p35 subdomain includes a sequence with at least 80% identity to any one of the sequences of SEQ ID NOs: 21 or 22. In some embodiments, the subject is mammalian and/or human. In some embodiments, the cancer is pancreatic cancer, prostate cancer, breast cancer, ovarian cancer, colorectal cancer, lung cancer, renal cell carcinoma, bladder cancer, medulloblastoma or neuroblastoma.

Also disclosed herein is a composition including a modified IL-12 protein construct. In some embodiments, the modified IL-12 protein construct includes a p35 subdomain, a linker, and a p40 subdomain. In some embodiments, the linker is 4, 5, 6, 7, 8, 9, 10, 11, 12, 13, 14, or 15 amino acids in length. In some embodiments, the p35 subdomain is on the C terminus, and the p40 subdomain is on the N terminus. In some embodiments, the p35 subdomain is on the N terminus, and the p40 subdomain is on the C terminus. In some embodiments, the linker includes a sequence with at least 80% identity to any one of the sequences of SEQ ID NOs: 23-26. In some embodiments, the p40 subdomain includes a sequence with at least 80% identity to any one of the sequences of SEQ ID NOs: 19 or 20. In some embodiments, the p40 subdomain further includes one, two, three, or four mutations. In some embodiments, the mutations include mutations of E81A, F82A, K106A, or K219A. In some embodiments, the p35 subdomain includes a sequence with at least 80% identity to any one of the sequences of SEQ ID NOs: 21 or 22. In some embodiments, the composition further includes a cell. In some embodiments, the cell is a T lymphocyte or a cytotoxic T lymphocyte.

Also disclosed herein is a pharmaceutical composition including an effective carrier and a modified IL-12 protein construct. In some embodiments, the modified IL-12 protein construct includes a p35 subdomain, a linker, and a p40 subdomain. In some embodiments, the linker is 4, 5, 6, 7, 8, 9, 10, 11, 12, 13, 14, or 15 amino acids in length. In some embodiments, the p35 subdomain is on the C terminus, and the p40 subdomain is on the N terminus. In some embodiments, the p35 subdomain is on the N terminus, and the p40 subdomain is on the C terminus. In some embodiments, the linker includes a sequence with at least 80% identity to any one of the sequences of SEQ ID NOs: 23-26. In some embodiments, the p40 subdomain includes a sequence with at least 80% identity to any one of the sequences of SEQ ID NOs: 19 or 20. In some embodiments, the p40 subdomain further includes one, two, three, or four mutations. In some embodiments, the mutations include mutations of E81A, F82A, K106A, or K219A. In some embodiments, the p35 subdomain includes a sequence with at least 80% identity to any one of the sequences of SEQ ID NOs: 21 or 22. In some embodiments, the composition further includes a cell. In some embodiments, the cell is a T lymphocyte or a cytotoxic T lymphocyte.

Also disclosed herein is method of treating a disease or disorder in a subject. In some embodiments, the method includes administering to the subject a modified IL-12 protein construct including a p35 subdomain, a linker, and a p40 subdomain. In some embodiments, the linker is 4, 5, 6, 7, 8, 9, 10, 11, 12, 13, 14, or 15 amino acids in length.

Also disclosed herein is a method of treating cancer in a subject, the method including administering to the subject a cell expressing a modified IL-12 protein construct including a p35 subdomain, a linker, and a p40 subdomain. In some embodiments, the linker is 4, 5, 6, 7, 8, 9, 10, 11, 12, 13, 14, or 15 amino acids in length. In some embodiments, the cell is a lymphocyte and/or leukocyte. In some embodiments, the cell is a T lymphocyte or a cytotoxic T lymphocyte.

Also disclosed herein is a composition including a modified IL-12 protein construct for use in the treatment of cancer. In some embodiments, the IL-12 protein construct includes a p35 subdomain, a linker, and a p40 subdomain. In some embodiments, the linker is 4, 5, 6, 7, 8, 9, 10, 11, 12, 13, 14, or 15 amino acids in length.

Also disclosed herein is a cell expressing a modified IL-12 protein construct for use in the treatment of cancer. In some embodiments, the IL-12 protein construct includes a p35 subdomain, a linker, and a p40 subdomain. In some embodiments, the linker is 4, 5, 6, 7, 8, 9, 10, 11, 12, 13, 14, or 15 amino acids in length. In some embodiments, the cell is a lymphocyte and/or leukocyte. In some embodiments, the cell is a T lymphocyte or a cytotoxic T lymphocyte.

### BRIEF DESCRIPTION OF THE DRAWINGS

In order to describe the manner in which the above-recited and other advantages and features of embodiments described herein can be obtained, a more particular description will be rendered by reference to specific embodiments thereof which are illustrated in the appended drawings. Understanding that these drawings depict only typical embodiments and are not therefore to be considered to be limiting of its scope, the disclosure will be described and explained with additional specificity and detail through the use of the accompanying drawings in which:

FIG. 1A depicts a nonlimiting example schematic of a CAR T-cell secreting wild type IL-12.

FIG. 1B depicts a nonlimiting example schematic of a CAR T-cell secreting a modified IL-12 protein of any one of the embodiments disclosed herein.

FIG. 2A depicts a non-limiting example schematic of a wild type IL-12.

FIG. 2B depicts a non-limiting example schematic of a modified IL-12 protein of any one of the embodiments disclosed herein. The circles shown in FIG. 2B represent mutations in IL-12 domains that can impact affinity to receptors, stability, or half-life.

FIG. 3A depicts a non-limiting schematic representation of a typical dose versus response relationship for wild type IL-12. Due to the toxicity of IL-12 when expressed at high levels, a low expression/low potency molecule is desired that facilitates activity while not exhibiting unwanted toxicity.

FIG. 3B depicts a non-limiting cartoon schematic representation of a wild type IL-12 composition and receptor usage. The cytokine-binding homology region (CHR) including tandem FNIII domains is shown with a double line in the upper domain and a single line in the lower domain.

FIG. 4A depicts SDS PAGE analysis of an scIL-12 construct that includes a p40 domain, a 15 amino acid linker, and a p35 domain (SEQ ID NO: 1).

FIG. 4B depicts Western blot analysis of an scIL-12 construct that includes a p40 domain, a 15 amino acid linker, and a p35 domain (SEQ ID NO: 1).

FIG. 4C depicts SDS PAGE analysis of an scIL-12 construct that includes a p40 domain, a 7 amino acid linker, and a p35 domain (SEQ ID NO: 2).

FIG. 4D depicts SDS PAGE analysis of an scIL-12 construct that includes a p35 domain, a 7 amino acid linker, and a p40 domain (SEQ ID NO: 5).

FIG. 4E depicts SDS PAGE analysis of an scIL-12 construct that includes a p35 domain, a 15 amino acid linker, and a p40 domain (SEQ ID NO: 3).

FIG. 5A depicts a non-limiting cartoon schematic representation of the IL-12 p40 and p35 domains.

FIG. 5B depicts a non-limiting cartoon schematic representation of a scIL-12 construct that includes p40, a 15 amino acid spacer, and p35 (SEQ ID NO: 1). As demonstrated in this cartoon schematic, the linker length in this orientation does not impact protein fold.

FIG. 5C depicts a non-limiting cartoon schematic representation of a scIL-12 construct that includes p40, a 7 amino acid spacer, and p35 (SEQ ID NO: 2). As demonstrated in this cartoon schematic, the linker length in this orientation does not impact protein fold.

FIG. 5D depicts a non-limiting cartoon schematic representation of a scIL-12 construct that includes p35, a 15 amino acid spacer, and p40 (SEQ ID NO: 3). As demonstrated in this cartoon schematic, the linker length in this orientation impacts protein fold.

FIG. 5E depicts a non-limiting cartoon schematic representation of a scIL-12 construct that includes p35, a 7 amino acid spacer, and p40 (SEQ ID NO: 5). As demonstrated in this cartoon schematic, the linker length in this orientation impacts protein fold.

FIG. 6 depicts a non-limiting cartoon schematic representation of a cell-based reporter assay.

FIG. 7 depicts a representative graph of the cell-based reporter assay as shown in FIG. 6, the scIL-12 variant constructs are assessed for their signaling capacity, as quantified by their ability to induce expression of alkaline phosphatase through the reporter gene STAT4.

FIG. 8 depicts a non-limiting cartoon schematic representation of an assay used for detecting scIL-12 secretion from T-cells.

FIG. 9 depicts a representative graph of GFP signaling given off in cells exposed to expressed scIL-12. In some embodiments, this graph is a marker for scIL-12 expression and secretion in cells.

FIG. 10 depicts a representative graph of the signaling capacity of scIL12 variants observed over 24, 48, and 72 hours after expression in Jurkat cells.

FIG. 11 depicts a representative graph of the signaling capacity of scIL12 variants observed over 24, 48, and 72 hours after expression in primary T cells.

FIG. 12 depicts a representative graph of the signaling capacity of scIL12 variants with varying linker lengths, observed over 24, 48, and 72 hours after expression in Jurkat cells.

FIG. 13A depicts a representative bar graph of phosphorylated STAT4 (pSTAT4) levels measured with an intracellular stain, following 96 hours of co-culturing NK cells with T cells expressing scIL-12 variants.

FIG. 13B depicts a representative bar graph of IFN-γ levels measured with an intracellular stain, following 96 hours of co-culturing NK cells with T cells expressing scIL-12 variants.

FIG. 13C depicts a representative bar graph of IFN-γ levels measured with ELISA of the co-culture media, following 96 hours of co-culturing NK cells with T cells expressing scIL-12 variants.

FIG. 14 depicts a non-limiting cartoon schematic representation of an assay used to measure pSTAT4 concentrations in T cells, as described in Example 7.

FIG. 15A depicts a representative bar and whiskers graph of intracellular pSTAT4 levels, following 30 minutes of culturing T cells with scIL variants.

FIG. 15B depicts a representative bar and whiskers graph of intracellular IFN-γ levels, following 24 hours of culturing T cells with scIL variants.

FIG. 16 depicts a representative bar and whiskers graph of intracellular pSTAT4 levels, following 30 minutes of culturing NK cells with scIL variants.

FIG. 17A depicts a representative graph of pSTAT4 levels produced by CD8⁺ primary CAR-T cells following exposure to scIL-12 p35-7aa-p40.

FIG. 17B depicts a representative graph of IFN-γ levels produced by CD4⁺ primary CAR-T cells following exposure to scIL-12 p35-7aa-p40.

FIG. 18 depicts a representative flow cytometry plot for GD2 staining in control cells (left curve), and B16.F10 melanoma cell lines engineered to express the enzymes GD2 and GD3 synthases (right curve).

FIGS. 19A-19B depict representative graphs for the change in tumour volume over time in mice transduced with CAR cells through intravenous (FIG. 19A) or intraperitoneal (FIG. 19B) administration. Mice were either non-transduced (“NT”), transduced with CD2 CAR cells, or by GD2 CAR in combination with various scIL-12 constructs (for example, p40-15aa-p35, 3Ala p40-15aa-p35, or p35-7aa-p40).

FIGS. 20A-20B depict representative graphs for the change of body weight in mice following CAR transduction through intravenous (FIG. 20A) or intraperitoneal (FIG. 20B) administration. Mice were either non-transduced (“NT”), transduced with CD2 CAR cells, or by GD2 CAR in combination with various scIL-12 constructs (for example, p40-15aa-p35, 3Ala p40-15aa-p35, or p35-7aa-p40).

### DETAILED DESCRIPTION

Although the disclosure is described in various exemplary alternatives and implementations as provided herein, it should be understood that the various features, aspects, and functionality described in one or more of the individual alternatives are not limited in their applicability to the particular alternative with which they are described. Instead, they can be applied alone or in various combinations to one or more of the other alternatives of the present disclosure, whether the alternatives are described or whether the features are presented as being a part of the described alternative. The breadth and scope of the present disclosure should not be limited by any exemplary alternatives described or shown herein.

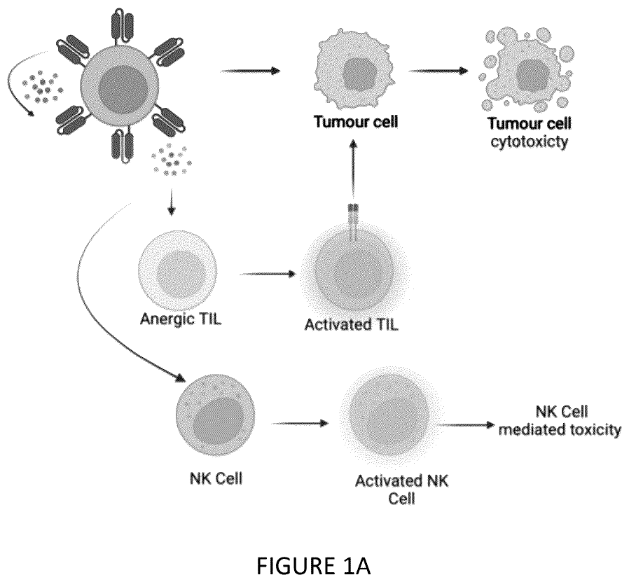

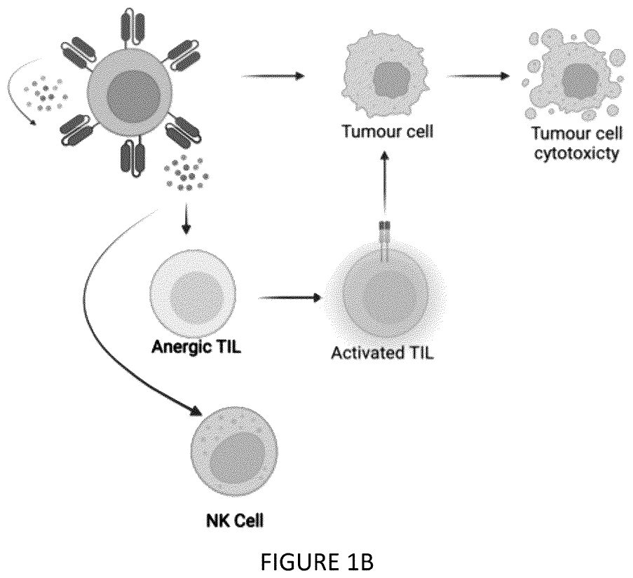

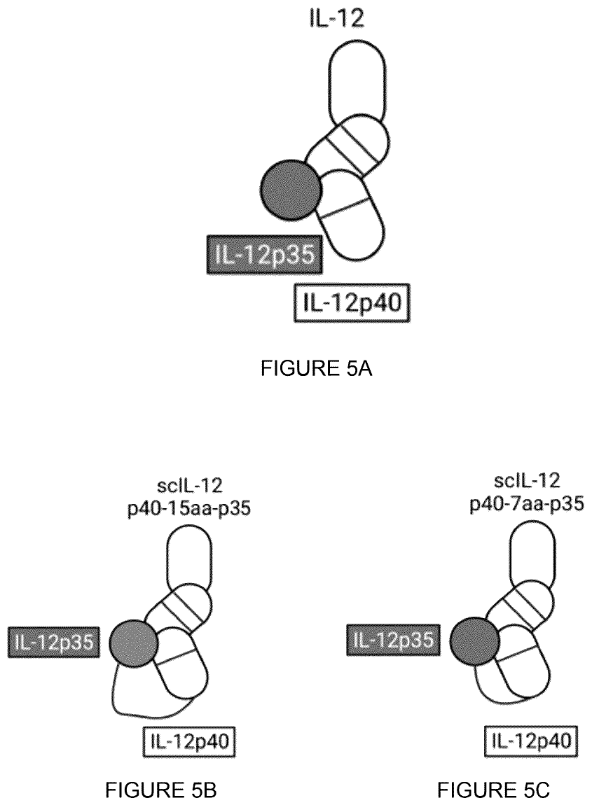

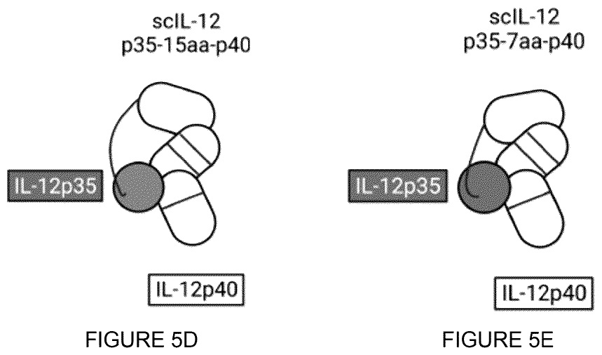

Disclosed herein is an IL-12 modified protein that is secreted from CAR T-cells to enhance the immunological effects of the cells and to modify the toxic metabolic encephalopathy (TME) (FIG. 1A-1B). In some embodiments, the IL-12 modified protein is a single chain version of IL-12 (“scIL-12”). In some embodiments, the IL-12 modified protein includes a p40 and a p35 subdomain (FIG. 5A). In some embodiments, the IL-12 modified protein has been engineered to optimize the affinity between cytokine and receptor. In some embodiments, the IL-12 modified protein has been engineered to reduce affinity to IL-12 receptors. In some embodiments, the IL-12 modified protein has been engineered to reduce its stability and/or half-life. In some embodiments, the IL-12 modified protein has been engineered to reduce its expression levels in a cell, tissue, organ, blood, plasma, and/or subject. In some embodiments, the IL-12 modified protein includes at least 1, 2, 3, 5, or 10 sequence mutations compared to the wildtype IL-12 protein. In some embodiments, the IL-12 modified protein includes at least one mutation at the interface of the interaction with IL-12 receptors. In some embodiments, the IL-12 modified protein includes at least one mutation in its p40 subunit. In some embodiments, the IL-12 modified protein includes at least one mutation in its p35 subunit.

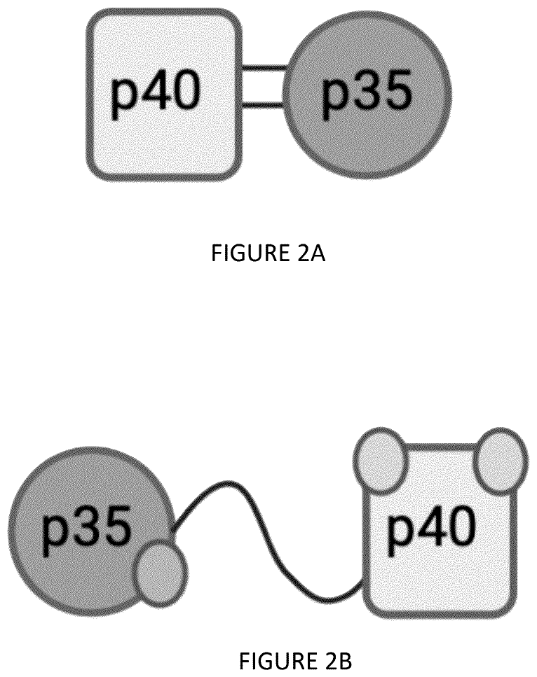

In some embodiments, the IL-12 modified protein is a single contiguous polypeptide chain. In some embodiments, the IL-12 modified protein has an optimized orientation. In some embodiments, the IL-12 modified protein has an optimized linker length. In some embodiments, the IL-12 modified protein has an optimized linker length between its p40 and p35 subunits (FIG. 2A-2B).

In some embodiments, the IL-12 modified protein has been engineered to provide the optimal therapeutic response of IL-12 while reducing the toxic effects of the molecule.

Some embodiments provided herein relate to a modified IL-12 protein construct that includes a p35 subdomain, a linker, and a p40 subdomain. In some embodiments, the linker is 4, 5, 6, 7, 8, 9, 10, 11, 12, 13, 14, or 15 amino acids in length. In some embodiments, the linker is 4 amino acids in length. In some embodiments, the linker is 5 amino acids in length. In some embodiments, the linker is 6 amino acids in length. In some embodiments, the linker is 7 amino acids in length. In some embodiments, the linker is 8 amino acids in length. In some embodiments, the linker is 9 amino acids in length. In some embodiments, the linker is 10 amino acids in length. In some embodiments, the linker is 11 amino acids in length. In some embodiments, the linker is 12 amino acids in length. In some embodiments, the linker is 13 amino acids in length. In some embodiments, the linker is 14 amino acids in length. In some embodiments, the linker is 15 amino acids in length. In some embodiments, the linker includes a sequence with at least 70%, 75%, 80%, 85%, 90%, 95%, 99%, 100%, or any integer that is between 70% and 100%, identity to any one of the sequences of SEQ ID NOs: 23-26. In some embodiments, the linker includes a sequence with at least 70%, 75%, 80%, 85%, 90%, 95%, 99%, 100%, or any integer that is between 70% and 100%, identity to the sequences of SEQ ID NO: 23. In some embodiments, the linker includes a sequence with at least 70%, 75%, 80%, 85%, 90%, 95%, 99%, 100%, or any integer that is between 70% and 100%, identity to the sequences of SEQ ID NO: 24. In some embodiments, the linker includes a sequence with at least 70%, 75%, 80%, 85%, 90%, 95%, 99%, 100%, or any integer that is between 70% and 100%, identity to the sequences of SEQ ID NO: 25. In some embodiments, the linker includes a sequence with at least 70%, 75%, 80%, 85%, 90%, 95%, 99%, 100%, or any integer that is between 70% and 100%, identity to the sequences of SEQ ID NO: 26.

In some embodiments, the p35 subdomain is on the C terminus, and the p40 subdomain is on the N terminus. In some embodiments, the p35 subdomain is on the N terminus, and the p40 subdomain is on the C terminus. In some embodiments, the p40 subdomain includes a sequence with at least 70%, 75%, 80%, 85%, 90%, 95%, 99%, 100%, or any integer that is between 70% and 100%, identity to any one of the sequences of SEQ ID NOs: 19 or 20. In some embodiments, the p40 subdomain includes a sequence with at least 70%, 75%, 80%, 85%, 90%, 95%, 99%, 100%, or any integer that is between 70% and 100%, identity to the sequence of SEQ ID NO: 19. In some embodiments, the p40 subdomain includes a sequence with at least 70%, 75%, 80%, 85%, 90%, 95%, 99%, 100%, or any integer that is between 70% and 100%, identity to the sequence of SEQ ID NO: 20. In some embodiments, the p40 subdomain further includes one, two, three, or four, including mutations of E81A, F82A, K106A, and/or K219A. In some embodiments, the p40 subdomain further includes at least one mutation, including at least one of E81A, F82A, K106A, or K219A. In some embodiments, the p40 subdomain further includes at least two mutations, including two or more of E81A, F82A, K106A, and/or K219A. In some embodiments, the p40 subdomain further includes at least three mutations, including three or more of E81A, F82A, K106A, and/or K219A. In some embodiments, the p40 subdomain further includes at least four mutations, including mutation of E81A, F82A, K106A, and K219A.

In some embodiments, the p35 subdomain includes a sequence with at least 70%, 75%, 80%, 85%, 90%, 95%, 99%, 100%, or any integer that is between 70% and 100%, identity to any one of the sequences of SEQ ID NOs: 21 or 22. In some embodiments, the p35 subdomain includes a sequence with at least 70%, 75%, 80%, 85%, 90%, 95%, 99%, 100%, or any integer that is between 70% and 100%, identity to the sequence of SEQ ID NO: 21. In some embodiments, the p35 subdomain includes a sequence with at least 70%, 75%, 80%, 85%, 90%, 95%, 99%, 100%, or any integer that is between 70% and 100%, identity to the sequence of SEQ ID NO: 22.

Some embodiments disclose a modified IL-12 protein construct. In some embodiments, the construct includes a sequence with at least 70%, 75%, 80%, 85%, 90%, 95%, 99%, 100%, or any integer that is between 70% and 100%, identity to any one of the sequences of SEQ ID NOs: 1-9. In some embodiments, the construct includes a sequence with at least 70%, 75%, 80%, 85%, 90%, 95%, 99%, 100%, or any integer that is between 70% and 100%, identity to the sequence of SEQ ID NO: 1. In some embodiments, the construct includes a sequence with at least 70%, 75%, 80%, 85%, 90%, 95%, 99%, 100%, or any integer that is between 70% and 100%, identity to the sequence of SEQ ID NO: 2. In some embodiments, the construct includes a sequence with at least 70%, 75%, 80%, 85%, 90%, 95%, 99%, 100%, or any integer that is between 70% and 100%, identity to the sequence of SEQ ID NO: 3. In some embodiments, the construct includes a sequence with at least 70%, 75%, 80%, 85%, 90%, 95%, 99%, 100%, or any integer that is between 70% and 100%, identity to the sequence of SEQ ID NO: 4. In some embodiments, the construct includes a sequence with at least 70%, 75%, 80%, 85%, 90%, 95%, 99%, 100%, or any integer that is between 70% and 100%, identity to the sequence of SEQ ID NO: 5. In some embodiments, the construct includes a sequence with at least 70%, 75%, 80%, 85%, 90%, 95%, 99%, 100%, or any integer that is between 70% and 100%, identity to the sequence of SEQ ID NO: 6. In some embodiments, the construct includes a sequence with at least 70%, 75%, 80%, 85%, 90%, 95%, 99%, 100%, or any integer that is between 70% and 100%, identity to the sequence of SEQ ID NO: 7. In some embodiments, the construct includes a sequence with at least 70%, 75%, 80%, 85%, 90%, 95%, 99%, 100%, or any integer that is between 70% and 100%, identity to the sequence of SEQ ID NO: 8. In some embodiments, the construct includes a sequence with at least 70%, 75%, 80%, 85%, 90%, 95%, 99%, 100%, or any integer that is between 70% and 100%, identity to the sequence of SEQ ID NO: 9. In some embodiments, the IL-12 protein construct further includes 1, 2, 3, or 4 mutations, including mutations of E81A, F82A, K106A, and/or K219A. In some embodiments, the p40 subdomain of the IL-12 protein construct further includes at least one mutation, including a mutation of E81A, F82A, K106A, or K219A. In some embodiments, the p40 subdomain further includes at least two mutations, including at least two mutations of E81A, F82A, K106A, and/or K219A. In some embodiments, the p40 subdomain further includes at least three mutations, including at least three mutations of E81A, F82A, K106A, and/or K219A. In some embodiments, the p40 subdomain further includes at least four mutations, including at least four mutations of E81A, F82A, K106A, and K219A.

Some embodiments disclose a nucleotide sequence encoding any one of the modified IL-12 protein constructs of any one of the embodiments of the present application.

Some embodiments disclose a nucleotide sequence expressing a modified IL-12 protein construct. In some embodiments, the nucleotide sequence includes a sequence with at least 70%, 75%, 80%, 85%, 90%, 95%, 99%, 100%, or any integer that is between 70% and 100%, identity to any one of the sequences of SEQ ID NOs: 10-18. In some embodiments, the nucleotide sequence includes a sequence with at least 70%, 75%, 80%, 85%, 90%, 95%, 99%, 100%, or any integer that is between 70% and 100%, identity to the sequences of SEQ ID NO: 10. In some embodiments, the nucleotide sequence includes a sequence with at least 70%, 75%, 80%, 85%, 90%, 95%, 99%, 100%, or any integer that is between 70% and 100%, identity to the sequences of SEQ ID NO: 11. In some embodiments, the nucleotide sequence includes a sequence with at least 70%, 75%, 80%, 85%, 90%, 95%, 99%, 100%, or any integer that is between 70% and 100%, identity to the sequences of SEQ ID NO: 12. In some embodiments, the nucleotide sequence includes a sequence with at least 70%, 75%, 80%, 85%, 90%, 95%, 99%, 100%, or any integer that is between 70% and 100%, identity to the sequences of SEQ ID NO: 13. In some embodiments, the nucleotide sequence includes a sequence with at least 70%, 75%, 80%, 85%, 90%, 95%, 99%, 100%, or any integer that is between 70% and 100%, identity to the sequences of SEQ ID NO: 14. In some embodiments, the nucleotide sequence includes a sequence with at least 70%, 75%, 80%, 85%, 90%, 95%, 99%, 100%, or any integer that is between 70% and 100%, identity to the sequences of SEQ ID NO: 15. In some embodiments, the nucleotide sequence includes a sequence with at least 70%, 75%, 80%, 85%, 90%, 95%, 99%, 100%, or any integer that is between 70% and 100%, identity to the sequences of SEQ ID NO: 16. In some embodiments, the nucleotide sequence includes a sequence with at least 70%, 75%, 80%, 85%, 90%, 95%, 99%, 100%, or any integer that is between 70% and 100%, identity to the sequences of SEQ ID NO: 17. In some embodiments, the nucleotide sequence includes a sequence with at least 70%, 75%, 80%, 85%, 90%, 95%, 99%, 100%, or any integer that is between 70% and 100%, identity to the sequences of SEQ ID NO: 18.

Some embodiments disclose a vector encoding any one of the modified IL-12 protein constructs of the present disclosure, and/or any one of the nucleotide sequences of the present disclosure.

Some embodiments disclose a cell containing any one of the modified IL-12 protein constructs of the pending disclosure, any one of the nucleotide sequences of the pending disclosure, and/or the vector of the pending disclosure. In some embodiments, the cell expresses the modified IL-12 protein. In some embodiments, the cell is a lymphocyte or leukocyte. In some embodiments, the cell is a T lymphocyte or a cytotoxic T lymphocyte. In some embodiments, the modified IL-12 protein has a reduced affinity for IL-12 receptors compared to wild type IL-12. In some embodiments, the modified IL-12 protein has a reduced stability and/or half-life compared to wild type IL-12. In some embodiments, the modified IL-12 protein has a reduced expression compared to wild type IL-12. In some embodiments, the reduced expression is monitored through the reduced concentration of IL-12 in the cell, tissue, organ, blood, plasma, system and/or subject. In some embodiments, the cell produces IFNγ compared to a cell expressing wild type IL-12.

Some embodiments disclose a method of treating a disease or disorder in a subject. In some embodiments, the disease or disorder is a cancer. In some embodiments, the disease or disorder is an infectious disease. In some embodiments, the disease or disorder is tuberculosis. In some embodiments, the disease or disorder is a viral infection. In some embodiments, the disease or disorder is hepatitis B or C. In some embodiments, the method includes administering to the subject the cell of any one of the cells of the present disclosure. In some embodiments, the subject is mammalian and/or human. In some embodiments, the cancer is selected from: renal cell carcinoma, bladder cancer, medulloblastoma, neuroblastoma, glioblastoma, mesothelioma, gastric cancer, blood cancers, lymphomas, multiple myeloma, leukemia diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), lung cancer, ovarian cancer, breast cancer, prostate cancer, liver cancer, kidney cancer, stomach cancer, pancreatic cancer, colorectal cancer, and colon cancer.

Chimeric antigen receptor (CAR)-based immunotherapy represents a promising therapeutic approach to multiple solid tumors, but it is often impeded by highly immunosuppressive tumor microenvironments (TME). Concomitant secretion of IL-12 by CAR T-cells may enable durable anti-tumor responses by boosting the cytotoxicity of CAR T-cells and further reshaping the TME, driving increased infiltration of proinflammatory CD4+ T-cells, decreased numbers of regulatory T-cells (Treg), and activation of the myeloid compartment. Importantly, engineering of the cytokine IL-12 will enable the immunotherapy-enabling benefits to be realized with minimal systemic effects that may lead to toxicity.

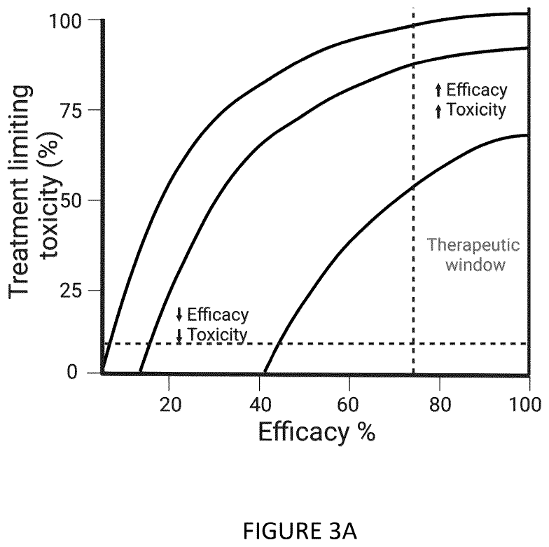

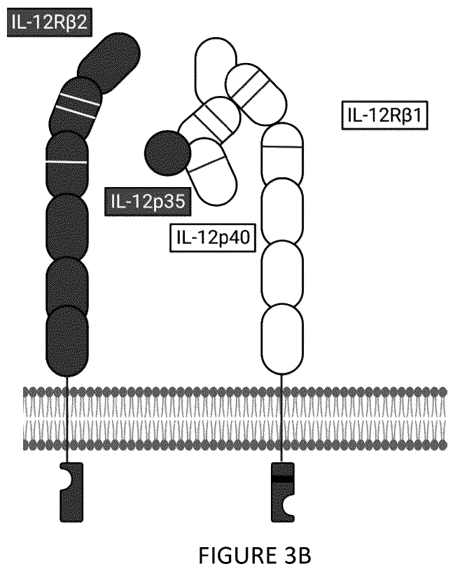

In some embodiments, the CAR T-cell secreting the modified IL-12 results in an improved antitumor efficacy while reducing the potentially toxic side effects of the cytokine driven by other immune cells such as NK cells (FIG. 3A). By utilizing a single chain molecule IL-12 can be precisely secreted from CAR T-cells that can be administered to patients while the potentially toxic effects are reduced through the engineering of the single chain construct and mutations of the individual domains of the polypeptide chain.

Wild type IL-12 is as shown in FIG. 3B. IL-12R β1 binds to the “back” of the IL-12 p40 subdomain, at the intersection between the N-terminal D1 Ig and D2 fibronectin domains of p40. IL-12R β1 binds p40 in a single, 1,532-A ° 2 interface that is characterized by a high degree of charge complementarity between the interacting proteins. Restricting the flexibility of the p40 protein may constrain it in such a way that the contiguous binding region to which the receptor interacts is interrupted.

### Definitions

The following definitions are provided to facilitate understanding of the alternatives or alternatives described herein.

As used herein, “a” or “an” may mean one or more.

As used herein, “about” in reference to a numeric value, including, for example, whole numbers, fractions, and percentages, generally refers to a range of numerical values (e.g., +/−5% to 10% of the recited value) that one of ordinary skill in the art would consider equivalent to the recited value (e.g., having the same function or result).

As used herein, “wild type” refers to how an entity occurs in nature. For example, “wild type IL-12” refers to the native, unmutated IL-12 that is expressed naturally in a subject. In some embodiments, “wild type IL-12” refers to the unmutated IL-12 that is expressed naturally in humans.

“IL-12” is given its standard scientific meaning, and thus refers to interleukin 12. In some embodiments, IL-12 is a heterodimeric cytokine encoded by two separate genes, IL-12A (p35) and IL-12B (p40). The active heterodimer, sometimes referred to as p70, and a homodimer of p40 are formed following protein synthesis. Native IL-12A (p35) is composed of four alpha helices, while native IL-12B (p40) is composed of three beta sheets.

“Chimeric antigen receptors” (CARs), as described herein, refers to genetically engineered protein receptors, which can confer specificity onto an immune effector cell, such as for example, a T-cell. Without being limiting, the use of CAR bearing T-cells can promote in vivo expansion and activation. The CARs can also be designed to redirect T-cells to target cells that express specific cell-surface antigens, where they can activate lymphocytes, such as T-cells, upon target recognition. The CARs graft the specificity of a monoclonal antibody or binding fragment thereof or scFv onto a T-cell, with the transfer of their coding sequence facilitated by vectors. In order to use CARs as a therapy for a subject in need, a technique called adoptive cell transfer is used in which T-cells are removed from a subject and modified so that they can express the CARs that are specific for an antigen. The T-cells, which can then recognize and target an antigen, are reintroduced into the patient. In some alternatives, CAR expressing lymphocytes are described. In some embodiments, the CAR expressing lymphocyte can be delivered to a subject to target specific cells. A TcR is a molecule on the surface of T lymphocytes or T-cells that can recognize antigens. As described herein, the CAR promotes in vivo expansion and activation of effector cells.

The structure of the CAR can include fusions of single-chain variable fragments (scFv) that are derived from monoclonal antibodies that are attached to transmembrane and cytoplasmic signaling domains. Most CARs can include an extracellular scFv that is linked to an intracellular CD32 domain (first generation CAR). Additionally, the scFv can be linked to a co-stimulatory domain, which can increase their efficacy in the therapy of a subject in need (second generation CAR). When T-cells express this molecule, they can recognize and kill target cells that express a specific antigen targeted by the CAR.

The chimeric antigen receptor can include a binding portion that is specific for a ligand. Without being limiting, the binding portion can include an antibody or binding fragment thereof or scFv, a receptor ligand or mutants thereof, peptide, and/or polypeptide affinity molecule or binding partner. In some alternatives of the first chimeric antigen receptor, the first chimeric antigen receptor includes a binding portion. In some embodiments, the binding portion includes an antibody or binding fragment thereof or scFv, a receptor ligand or mutants thereof, peptide, and/or polypeptide affinity molecule or binding partner. In some alternatives, the binding portion is specific for a ligand on a B-cell. In some alternatives of the second chimeric antigen receptor, the second chimeric antigen receptor includes a binding portion. In some embodiments, the binding portion includes an antibody or binding fragment thereof or scFv, a receptor ligand or mutants thereof, peptide, and/or polypeptide affinity molecule or binding partner. In some alternatives, the binding portion is specific for a ligand on a tumor cell. In some alternatives, the tumor is not a tumor of a B-cell related cancer.

“Ligand” as described herein, refers to a substance that can form a complex with a biomolecule. By way of example and not of limitation, ligands can include substrates, proteins, small molecules, inhibitors, activators, nucleic acids and neurotransmitters. Binding can occur through intermolecular forces, for example ionic bonds, hydrogen bonds, and van der walls interactions. Ligand binding to a receptor protein can alter the three dimensional structure and determine its functional state. The strength of binding of a ligand is referred to as the binding affinity and can be determined by direct interactions and solvent effects. A ligand can be bound by a “ligand binding domain.” A ligand binding domain, for example, can refer to a conserved sequence in a structure that can bind a specific ligand or a specific epitope on a protein. The ligand binding domain or ligand binding portion can include an antibody or binding fragment thereof or scFv, a receptor ligand or mutants thereof, peptide, and/or polypeptide affinity molecule or binding partner. Without being limiting, a ligand binding domain can be a specific protein domain or an epitope on a protein that is specific for a ligand or ligands.

In some alternatives, a chimeric antigen receptor is provided. In some embodiments, the ligand or target molecule is a cell surface molecule that is found on tumor cells and is not substantially found on normal tissues, or restricted in its expression to non-vital normal tissues. In some alternatives, the tumor does not originate from a B-cell related cancer. In some alternatives, the ligand or target molecule is found on a tumor cell as well as on normal tissues. In some alternatives the cells expressing a CAR that is specific for a ligand on tumor cells and normal tissue further includes a suicide gene to limit the time of therapy and increase their safety profile. Conditional suicide genes may also be applied to the donor T-cells to limit the attack on normal tissue that may express a tumor associated antigen or ligand.

“Effector cells” as described herein, refers to a lymphocyte that has been induced to differentiate into another cell type that can be capable of mounting a specific immune response, such as a terminally differentiated leukocyte that performs one or more specific functions. The main effector cells of the immune system, for example, are activated lymphocytes and phagocytes that are involved in destroying pathogens and removing them from the body. The effector cells can include large granular lymphocytes, such as, for example, natural killer cells and cytotoxic T lymphocytes. In some alternatives of the cells provided herein, the cell includes a first and second chimeric antigen receptor. In some embodiments, the first chimeric antigen receptor is specific for a ligand on a B-cell, which promotes the in vivo expansion and activation of an effector cell. In some embodiments, the second chimeric antigen receptor is specific for a ligand on a tumor. In some alternatives, the cells that undergo expansion and activation are lymphocytes, phagocytes, large granular lymphocytes, natural killer cells and/or cytotoxic T lymphocytes.

“Cancer antigen,” “tumor antigen” or “tumor marker” refers to an antigenic substance that is produced in a tumor cell, which can therefore trigger an immune response in the host. These cancer antigens can be useful as markers for identifying a tumor cell, which will be a potential candidate during treatment or therapy. There are several types of cancer or tumor antigens. There are tumor specific antigens (TSA) which are present only on tumor cells and not on healthy cells, as well as tumor associated antigens (TAA) which are present in tumor cells and also on some normal cells. In some alternatives of the methods and chimeric antigens provided herein, the chimeric antigen receptors are specific for tumor specific antigens. In some alternatives, the chimeric antigen receptors are specific for tumor associated antigens. In some alternatives described herein, the tumor does not originate from a B-cell related cancer. In some alternatives, cells expressing a CAR that is specific for a TAA is further modified by the introduction of a suicide gene to limit the time of the CAR T-cell therapy and to reduce the attack of normal tissues expressing the TAA.

In some alternatives of the methods provided herein, the cancer antigen is EGFR, HER2, Mesothelin, cancer testis antigens, L1CAM, o-acetylated GD2, GD2, neoantigens, Var2, glypican-2 (GPC2), HPV antigens, alpha fetoprotein, carcinoembryonic antigen, CA-125, MUC-1, epithelial tumor antigen, abnormal products of Ras or p53, EphA2, MAGE-A3, MAGE-A4, MAGE-C2, PRAME, SSX2, adipophilin, AIM2, ALDH1A1, BCLX, EpCAM, CS274, CPSF, cyclin D1, DKK1, ENAH, EpCAM, EphA3, EZH2, FGF5, glypican-3, G250, HLA-DOB, Hepsin, ID01, IGF2B3, IL13Ralpha2, Intestinal carboxylesterase, alpha-foetoprotein, kallikrein4, KIF20A, Lengsin, M-CSF, MCSP, mdm-2, Meloe, midkine, MMP-2, MMP-7, MUC1, MUC5AC, p53, PAX5, PBF, PRAME, PSMA, RAGE-1, RGS5, RhoC, RNF43, RUF43, FU2AS, secernin 1, SOX10, STEAP1, survivin, telomerase, TPBG, VEGF, WT1, NY-ESO-1 or ROR1. In some alternatives, the cell surface tumor specific molecule is ROR1. In some alternatives, the cancer antigen is expressed by a tumor. In some embodiments, the tumor is not a B-cell related cancer.

“Specific” or “Specificity” can refer to the characteristic of a ligand for the binding partner or alternatively, the binding partner for the ligand, and can include complementary shape, charge and hydrophobic specificity for binding. Specificity for binding can include stereospecificity, regioselectivity and chemoselectivity. In some alternatives, a chimeric antigen receptor is provided. In some embodiments, the chimeric antigen receptor is specific for a B-cell ligand. In some alternatives, a chimeric antigen receptor is provided. In some embodiments, the chimeric antigen receptor is specific for a tumor cell ligand.

A “regulatory element” as described herein, can refer to a regulatory sequence, which is any DNA sequence that is responsible for the regulation of gene expression, such as promoters and operators. The regulatory element can be a segment of a nucleic acid molecule which is capable of increasing or decreasing the expression of specific genes within an organism. In some alternatives described herein, a cell is provided. In some embodiments, the cell includes a first and second chimeric antigen receptor or TcR. In some embodiments, the first chimeric antigen receptor is specific for a ligand on a B-cell, which promotes the in vivo expansion and activation of an effector cell. In some embodiments, the second chimeric antigen receptor or TcR is specific for a ligand on a tumor. In some alternatives, the first chimeric antigen receptor and/or the second chimeric antigen receptor or TcR are inducibly expressed in said cell. In some alternatives, expression of the first chimeric antigen receptor and/or the second chimeric antigen receptor or TcR is under the control of a regulatory element.

“Transmembrane domain” as described herein is an integral protein that can span a cellular membrane.

A “promoter” is a nucleotide sequence that directs the transcription of a structural gene. In some alternatives, a promoter is located in the 5′ non-coding region of a gene, proximal to the transcriptional start site of a structural gene. Sequence elements within promoters that function in the initiation of transcription are often characterized by consensus nucleotide sequences. Without being limiting, these promoter elements can include RNA polymerase binding sites, TATA sequences, CAAT sequences, differentiation-specific elements (DSEs; McGehee et al., Mol. Endocrinol. 7:551 (1993); incorporated by reference in its entirety), cyclic AMP response elements (CREs), serum response elements (SREs; Treisman, Seminars in Cancer Biol. 1:47 (1990); incorporated by reference in its entirety), glucocorticoid response elements (GREs), and binding sites for other transcription factors, such as CRE/ATF (O'Reilly et al., J. Biol. Chem. 267:19938 (1992); incorporated by reference in its entirety), AP2 (Ye et al., J. Biol. Chem. 269:25728 (1994); incorporated by reference in its entirety), SP1, cAMP response element binding protein (CREB; Loeken, Gene Expr. 3:253 (1993); incorporated by reference in its entirety) and octamer factors (see, in general, Watson et al., eds., Molecular Biology of the Gene, 4th ed. (The Benjamin/Cummings Publishing Company, Inc. 1987; incorporated by reference in its entirety)), and Lemaigre and Rousseau, Biochem. J. 303:1 (1994); incorporated by reference in its entirety). As used herein, a promoter can be constitutively active, repressible or inducible. If a promoter is an inducible promoter, then the rate of transcription increases in response to an inducing agent. In contrast, the rate of transcription is not regulated by an inducing agent if the promoter is a constitutive promoter. Repressible promoters are also known. In some alternatives of the nucleic acid is provided, the nucleic acid includes a promoter sequence. In some alternatives of the chimeric antigen, the chimeric antigen is inducibly expressed in response to an inducing agent. In some alternatives, the TcR is inducibly expressed in response to an inducing agent.

In some alternatives, promoters used herein can be inducible or constitutive promoters. Without being limiting, inducible promoters can include, for example, a tamoxifen inducible promoter, tetracycline inducible promoter, and doxycycline inducible promoter (e.g. Tre) promoter. Constitutive promoters can include, for example, SV40, CMV, UBC, EF1 alpha, PGK, and CAGG. In some alternatives, the promoter is a regulatory promoter. In some alternatives, the promoter is a tamoxifen inducible promoter, a tetracycline inducible promoter, or a doxycycline inducible promoter (e.g. Tre) promoter. In some alternatives provided herein, expression of a chimeric antigen receptor or a TcR on a cell is induced by tamoxifen and/or its metabolites. Metabolites for tamoxifen are active metabolites such as 4-hydroxytamoxifen (afimoxifene) and N-desmethyl-4-hydroxytamoxifen (endoxifen), which can have 30-100 times more affinity with an estrogen receptor than tamoxifen itself. In some alternatives, the tamoxifen metabolites are 4-hydroxytamoxifen (afimoxifene) and/or N-desmethyl-4-hydroxytamoxifen (endoxifen). Some embodiments provided herein relate to vectors. In some embodiments, the vector has a first promoter for the CAR/TcR and a second promoter for the marker protein.

An “antibody” as described herein, refers to a large Y-shape protein produced by plasma cells that is used by the immune system to identify and neutralize foreign objects such as bacteria and viruses. The antibody protein can include four polypeptide chains; two identical heavy chains and two identical light chains connected by disulfide bonds. Each chain is composed of structural domains called immunoglobulin domains. These domains can contain about 70, 80, 90, 100, 110, 120, 130, 140, 150 amino acids or any number of amino acids in between in a range defined by any two of these values, and are classified into different categories according to their size and function. In some alternatives, the ligand binding domain includes an antibody or binding fragment thereof or scFv, a receptor ligand or mutants thereof, peptide, and/or polypeptide affinity molecule or binding partner. In some alternatives, the ligand binding domain is an antibody fragment, desirably, a binding portion thereof. In some alternatives, the antibody fragment or binding portion thereof present on a CAR is specific for a ligand on a B-cell. In some alternatives, the antibody fragment or binding portion thereof present on a CAR or TcR is specific for a ligand on a tumor cell. In some alternatives, the tumor is not derived from a B-cell related cancer. In some alternatives, the antibody fragment or binding portion thereof present on a CAR is specific for a ligand present on a tumor cell. In some alternatives, the ligand binding domain is an antibody fragment or a binding portion thereof, such as a single chain variable fragment (scFv). In some alternatives, the ligand includes a tumor specific mutation. In some alternatives, the antibody fragment or binding portion thereof present on a CAR includes one or more domains from a humanized antibody, or binding portion thereof.

As used herein, “polynucleotide,” “nucleic acid,” “nucleotides,” “nucleotide sequence,” “nucleic acid sequence,” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both.

Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which include naturally-occurring or modified nucleic acid bases attached to a polyamide backbone. The nucleic acid may be double stranded, single stranded, or contain portions of both double stranded or single stranded sequence. As will be appreciated by those in the art, the depiction of a single strand (“Watson”) also defines the sequence of the other strand (“Crick”). By the term “recombinant nucleic acid” herein is meant nucleic acid, originally formed in vitro, in general, by the manipulation of nucleic acid by endonucleases, in a form not normally found in nature. Thus, an isolated nucleic acid, in a linear form, or an expression vector formed in vitro by ligating DNA molecules that are not normally joined, are both considered recombinant for the purposes of this disclosure. It is understood that once a recombinant nucleic acid is made and reintroduced into a host cell or organism, it will replicate non-recombinantly, for example, using the in vivo cellular machinery of the host cell rather than in vitro manipulations; however, such nucleic acids, once produced recombinantly, although subsequently replicated non-recombinantly, are still considered recombinant for the purposes of the disclosure.

As used herein, “sequence identity” or “identity” in the context of two nucleic acid sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and, therefore, do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Any suitable means for making this adjustment may be used. This may involve scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (IntelliGenetics, Mountain View, Calif.).

As used herein, “percentage of sequence identity” means the value determined by comparing two optimally aligned sequences over a comparison window, the portion of the polynucleotide sequence in the comparison window may include additions or deletions (such as gaps) as compared to the reference sequence (which does not include additions or deletions) for optimal alignment of the two sequences. The percentage can be calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Any suitable methods of alignment of sequences for comparison may be employed. Thus, the determination of percent identity between any two sequences can be accomplished using a mathematical algorithm. Preferred, non-limiting examples of such mathematical algorithms are the algorithm of Myers and Miller, CABIOS, 4:11 (1988), which is hereby incorporated by reference in its entirety; the local homology algorithm of Smith et al, Adv. Appl. Math., 2:482 (1981), which is hereby incorporated by reference in its entirety; the homology alignment algorithm of Needleman and Wunsch, J M B, 48:443 (1970), which is hereby incorporated by reference in its entirety; the search-for-similarity-method of Pearson and Lipman, Proc. Natl. Acad. Sci. USA, 85:2444 (1988), which is hereby incorporated by reference in its entirety; the algorithm of Karlin and Altschul, Proc. Natl. Acad. Sci. USA, 87:2264 (1990), which is hereby incorporated by reference in its entirety; modified as in Karlin and Altschul, Proc. Natl. Acad. Sci. USA, 90:5873 (1993), which is hereby incorporated by reference in its entirety.

Computer implementations of these mathematical algorithms can be utilized for comparison of sequences to determine sequence identity. Such implementations include, but are not limited to: CLUSTAL in the PC/Gene program (available from IntelliGenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988), Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994), which are hereby incorporated by reference in their entirety. The ALIGN program is based on the algorithm of Myers and Miller, supra. The BLAST programs of Altschul et al., JMB, 215:403 (1990); Nucl. Acids Res., 25:3389 (1990), which are hereby incorporated by reference in their entirety, are based on the algorithm of Karlin and Altschul supra.

The terms “polypeptide”, “peptide”, “protein,” and “protein construct” are used interchangeably herein to refer to polymers of amino acids of any length. The polymer may be linear, cyclic, or branched, it may include modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. Standard amino acids can be written in their full name, three letter name, or one letter name; for example: Histidine, His, or H. Non-limiting examples of amino acids include: histidine, lysine, methionine, phenylalanine, threonine, tryptophane, asparagine, aspartic acid/aspartate, alanine, arginine, cysteine, glutamic acid/glutamate, glutamine, glycine, proline, serine, and tyrosine. Thus, exemplary polypeptides or proteins include gene products, naturally occurring proteins, homologs, orthologues, paralogs, fragments and other equivalents, variants, fragments, and analogs of the foregoing.

As used herein the term “amino acid” has its ordinary meaning as understood in light of the specification refers to either natural and/or unnatural or synthetic amino acids, including glycine and both the D or L optical isomers, and amino acid analogs and peptidomimetics. For purposes of classifying amino acids substitutions as conservative or nonconservative, amino acids are grouped as follows: Group I (hydrophobic side chains): Met, Ala, Val, Leu, Ile; Group II (neutral hydrophilic side chains): Cys, Ser, Thr; Group III (acidic side chains): Asp, Glu; Group IV (basic side chains): Asn, Gln, His, Lys, Arg; Group V (residues influencing chain orientation): Gly, Pro; and Group VI (aromatic side chains): Trp, Tyr, Phe. Conservative substitutions involve substitutions between amino acids in the same class. Non-conservative substitutions constitute exchanging a member of one of these classes for a member of another.

Amino acid substitutions in a native protein sequence may be “conservative” or “non-conservative” and such substituted amino acid residues may or may not be one encoded by the genetic code. A “conservative amino acid substitution” is one in which the amino acid residue is replaced with an amino acid residue having a chemically similar side chain (for example, replacing an amino acid possessing a basic side chain with another amino acid with a basic side chain). A “non-conservative amino acid substitution” is one in which the amino acid residue is replaced with an amino acid residue having a chemically different side chain (for example, replacing an amino acid having a basic side chain with an amino acid having an aromatic side chain). The standard twenty amino acid “alphabet” is divided into chemical families based on chemical properties of their side chains. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and side chains having aromatic groups (e.g., tyrosine, phenylalanine, tryptophan, histidine).

A standard amino acid includes a backbone with an amine group on one end, and a carboxylic group on the opposite end. Peptide bonds are formed through the attachment of one amino acid's carboxylic group to a second amino acid's amino group. This results in a backbone sequence of “NCC—NCC—NCC . . . ” etc. An “N-terminal” amino acid is therefore an amino acid within a polypeptide that is at one end of the polypeptide, and is bonded to the polypeptide through its carboxylic acid group alone. In other words, the amine group of an N-terminal amino acid is not bound to another amino acid. Conversely, a “C-terminal” amino acid is a terminal amino acid within a polypeptide that is bonded to the rest of the polypeptide through its amino group. In other words, the carboxylic acid group of a C-terminal amino acid is not bound to another amino acid.

An “N-terminal domain” or “N-terminal subdomain,” is therefore a domain or subdomain of a protein, respectively, that includes the N-terminal amino acid, and the “C-terminal domain” or “C-terminal subdomain” is the domain or subdomain of a protein, respectively, that includes the C-terminal amino acid. In nature, proteins are synthesized from the N terminus to the C terminus, so sequences are typically written N to C. For example, if a given protein includes Domain 1 and Domain 2, and the sequence of the protein's amino acid backbone is “NCC—NCC—NCC . . . ,” then Domain 1 would be the N-terminal domain, and Domain 2 would be the C-terminal domain.

A polypeptide or amino acid sequence that is “modified” or “derived from” a designated protein refers to the origin of the polypeptide. Preferably, the polypeptide has an amino acid sequence that is essentially identical to that of a polypeptide encoded in the sequence, or a portion thereof. In some embodiments, the portion includes at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence. Peptide sequences having at least 80%, 85%, 90%, 95%, 99%, or 100% homology to any one of the peptide sequences disclosed herein and having the same or similar functional properties are envisioned. The percent homology may be determined according to amino acid substitutions, deletions, or additions between two peptide sequences. Peptide sequences having some percent homology to any one of the peptide sequences disclosed herein may be produced and tested by one skilled in the art through conventional methods. The % homology or % identity of two sequences is well understood in the art and can be calculated by the number of conserved amino acids or nucleotides relative to the length of the sequences.

A protein “domain” is a select region of a protein. A domain may be conserved through related proteins. In some embodiments, the protein domain is self-stabilizing and forms independently from the rest of the protein. For example, in an IL-12 protein, p35 and p40 are both considered subdomains of IL-12. A “subdomain” is a smaller, distinct region within a domain. For example, a region within a p35 sequence would be a p35 subdomain.

“Polymer” refers to a series of monomer groups linked together. A polymer is composed of multiple units of a single monomer (a homopolymer) or different monomers (a heteropolymer). High MW polymers are prepared from monomers that include, but are not limited to, acrylates, methacrylates, acrylamides, methacrylamides, styrenes, vinyl-pyridine, vinyl-pyrrolidone and vinyl esters such as vinyl acetate. Additional monomers are useful in high MW polymers. When two different monomers are used, the two monomers are called “comonomers,” meaning that the different monomers are copolymerized to form a single polymer. The polymer can be linear or branched. When the polymer is branched, each polymer chain is referred to as a “polymer arm.” The end of the polymer arm linked to the initiator moiety is the proximal end, and the growing-chain end of the polymer arm is the distal end. On the growing chain-end of the polymer arm, the polymer arm end group can be the radical scavenger, or another group.

The term “construct” refers to an artificial protein or nucleotide sequence. For example, a “protein construct” is an engineered protein. A protein construct includes at least one polypeptide sequences. In some embodiments, the construct includes a functional element.

A “chemical linker” or “linker” refers to a chemical moiety that links two groups together, such as a half-life extending moiety and a protein. The linker can be cleavable or non-cleavable. Cleavable linkers can be hydrolyzable, enzymatically cleavable, pH sensitive, photolabile, or disulfide linkers, among others. Other linkers include homobifunctional and heterobifunctional linkers. A “linking group” is a functional group capable of forming a covalent linkage including one or more bonds to a bioactive agent. In some embodiments, the linker includes a series of nucleotides and/or amino acids.

The term “reactive group” refers to a group that is capable of reacting with another chemical group to form a covalent bond, for example, is covalently reactive under suitable reaction conditions, and generally represents a point of attachment for another substance. The reactive group is a moiety, such as maleimide or succinimidyl ester, capable of chemically reacting with a functional group on a different moiety to form a covalent linkage. Reactive groups generally include nucleophiles, electrophiles and photoactivatable groups.

“Molecular weight” in the context of the polymer can be expressed as either a number average molecular weight, or a weight average molecular weight or a peak molecular weight. Unless otherwise indicated, all references to molecular weight herein refer to the peak molecular weight. These molecular weight determinations, number average (Mn), weight average (Mw) and peak (Mp), can be measured using size exclusion chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight, or the use of light scattering techniques, ultracentrifugation or viscometry to determine weight average molecular weight. In some embodiments, the molecular weight is measured by SEC-MALS (size exclusion chromatography-multi angle light scattering). In some embodiments, the polymeric reagents are typically polydisperse (for example, number average molecular weight and weight average molecular weight of the polymers are not equal), and can possess low polydispersity values of, for example, less than about 1.5, as judged, for example, by the PDI value derived from the SEC-MALS measurement. In some embodiments, the polydispersities (PDI) are in the range of about 1.4 to about 1.2. In some embodiments the PDI is less than about 1.15, 1.10, 1.05, or 1.03.

“Block” or “inhibit” has their ordinary meaning as understood in light of the specification, and refer to reducing or alleviating the functional activity of a protein. For example, if a protein is capable of activating a cellular signal, then blocking that function reduces or eliminates the activation of that cellular signal.

“Vector” as described herein, is a nucleic acid vehicle that carries a generic material encoding a protein or mRNA of interest into another cell, such that it is replicated and/or expressed in the cell. There are several types of vectors. Without being limiting, a vector can be a plasmid, viral vector, cosmid, artificial chromosome, or an mRNA. The vector can be linear or circular. In some alternatives provided herein, a viral vector is used to carry the nucleic acid encoding a chimeric antigen receptor. In some alternatives, the viral vector is a lentiviral vector. In some alternatives, the viral vector is a retroviral vector. In some embodiments, the viral vector is a gammaretroviral vector. In some alternatives, the vector is a foamy viral vector. In some alternatives, the vector is a plasmid. In some alternatives, the vector is an mRNA. In some alternatives, the vector is linear and includes telomeres.

An “expression cassette” as described herein, has its ordinary meaning as understood in light of the specification, and refers to a gene operatively linked to a regulatory sequence. Without being limiting, transduction or transfection of an expression cassette into a cell may result in the successful expression of the gene's encoded protein.

“Express” or “expression” as described herein have their ordinary meaning as understood in light of the specification, and thus refer to the presence of a molecule in a living system. For example, “gene expression” refers to the transcription and translation of a DNA gene into first an RNA, and then a protein. Similarly, “protein expression” refers to the synthesis, and subsequent presence, of a protein within a system. It will be therefore understood that if a cell is said to “express” protein A, that cell is capable of producing protein A.

“Plasmid” as described herein, is a genetic structure in a cell that can replicate independently of the chromosomes. Without being limiting, the plasmid can be a small circular DNA strand in the cytoplasm of a bacterium or protozoan, or a linear nucleic acid.

“Signaling domain” as described herein is a domain on a chimeric antigen receptor that can promote cytokine release, in vivo T-cell survival and tumor elimination. In some alternatives herein, a signaling domain includes CD28, 4-1BB and/or CD3-zeta cytoplasmic domains.

The term “cell” includes those of prokaryotes and eukaryotes, and may further include bacterial cells, mycobacteria cells, fungal cells, yeast cells, plant cells, insect cells, non-human animal cells, human cells, or cell fusions such as, for example, hybridomas. In some embodiments, the cell is eukaryotic. In some embodiments, the cell is a mammalian cell. In some embodiments, the cell is derived from human, monkey, ape, hamster, rat, or mouse cells. In some embodiments, the cell is human. In some embodiments, the cell is an immune cell. In some embodiments, the cell is a lymphocyte. In some embodiments, the cell is a T cell. In some embodiments, the cell is a tumor infiltrating lymphocyte (TIL) cell, a natural killer (NK) cell, a CD8+ T cell, a CD4+ T cell, a regulatory T cell, or a memory T cell.

“Immune cells” as described herein, have their ordinary meaning as understood in light of the specification, and refer to cells that are part of the immune system. In some embodiments, the cell is part of the innate immune system. In some embodiments, the cell is part of the adaptive immune system. Non-limiting example of immune cells include blood cells, bone marrow cells, hematopoietic stem cells, lymphoid progenitor cells, myeloid progenitor cells, B cell progenitors, memory B cells, plasma cells, monocytes, macrophages, dendritic cells, basophils, neutrophils, eosinophils, mast cells, natural killer cells, T cell progenitors, memory T cell, cytotoxic T cells, and helper T cells.

“Solid Tumors” as described herein, refers to a malignant cancerous mass of tissue. In some alternatives of the methods of treating, ameliorating, or inhibiting a non-B-cell related disease in a subject provided herein, the method includes introducing, providing, or administering any one or more of the cells or compositions of any of the alternatives herein or the cells made by any one or more of the methods of the alternatives herein into a subject for therapy. In some alternatives, the subject has a cancer. In some alternatives, the cancer is a solid tumor. In some alternatives, the solid tumor is a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and ovarian cancer.

“Engraftment” as described herein, refers to the incorporation of grafted tissue into the body of the host. Several characteristics of effective CAR T-cells include showing signs of adequate engraftment, which is required for responses. For example, detection of the CAR transgene by polymerase chain reaction is not informative about the surface expression of the CAR, which is the only form that matters for efficacy. Thus, the availability of reagents to specifically detect CARs at the cell surface by flow cytometry or other methods known to those skilled in the art is crucial to understand the activity and engraftment of CAR T-cells. In the alternatives described herein, the therapeutic potency of the adoptively transferred CARs are improved by allowing a B-cell targeting CAR to drive the activation, proliferation and dispersion of infused CAR T-cells that have a second CAR that provides for redirected killing of the solid tumor. In some alternatives described herein, the methods and cells include including a CAR with B-cell specificity led to the surprising effect of having an improved level of engraftment compared to T-cells that only included CARs specific for a tumor ligand. As described in the alternatives herein, the obstacle of failure to exhibit engraftment is overcome by allowing a B-cell targeting CAR to drive the activation, proliferation and dispersion of infused CAR T-cells that have a CAR that provides for redirected killing of the solid tumor.

A “cytokine” as described herein, has its ordinary meaning as understood in light of the specification, and is a small molecule that is secreted by one cell and that has an effect on other cells. Cytokines, sometime considered as “stress proteins,” include chemokines, interferons, interleukins, lymphokines, and tumor necrosis factors. Cytokines are produced by many cells, including macrophages, B lymphocytes, T lymphocytes and mast cells, as well as endothelial cells, fibroblasts, and various stromal cells. A particular cytokine may be produced by more than one type of cell. Non-limiting examples of cytokines include members of the IL-1 family, TNF family, interferons, IL-6 family, IL-10 family, TGF-beta family, and chemokines. Common cytokines include IL-1, IL-2, IL-3, IL-4, IL-5, IL-6, IL-8, IL-9, IL-10, IL-11, IL-12, IL-13, IL-15, IL-16, IL-18, IL-21, IL-33, IFN-alpha, IFN-beta, IFN-gamma, TNF-alpha, and TNF-beta.

“Cytokine signaling” as described herein, has its ordinary meaning as understood in light of the specification, and refers to the process by which a cytokine is recognized by a cytokine receptor on the surface of a cell, and elicits a response. These signals may either be “autocrine” (for example, where the same cell both produces the cytokine and responds to it) or “paracrine” (for example, where the cytokine is made by one cell and acts on another). Cytokine receptors are grouped into six major families: class I cytokine receptors, class II cytokine receptors, IL-1 receptors, TNF receptors, tyrosine kinase receptors, and chemokine receptors. Cytokines activate many pathways; for example, the JAK-STAT pathway. In this pathway, JAK proteins phosphorylate a cytokine receptor once that receptor binds to its corresponding cytokine. This newly phosphorylated residue on the cytokine receptor then acts as a binding site for a STAT protein. Once the STAT is bound, it is phosphorylated by JAK and forms a homodimer with another STAT. This complex then dissociates from the receptor, travels to the nucleus, and induces transcription of crucial genes.

“Subject” or “patient,” as described herein, refers to any organism upon which the alternatives described herein may be used or administered, e.g., for experimental, diagnostic, prophylactic, and/or therapeutic purposes. Subjects or patients include, for example, animals. In some alternatives, the subject is mice, rats, rabbits, non-human primates, and humans. In some alternatives, the subject is a cow, sheep, pig, horse, dog, cat, primate or a human. The terms, “individual,” “patient” and “subject” are used interchangeably herein.

As used herein, the terms “treat,” “treatment,” or “treating,” refer to therapeutic treatments, where the object is to reverse, alleviate, ameliorate, inhibit, slow down or stop the progression or severity of a condition, e.g., a disease or disorder. The term “treating” includes reducing or alleviating at least one adverse effect or symptom of a condition, disease or disorder associated with, e.g., arteriosclerosis, gingivitis, etc. Treatment is generally “effective” if one or more local or systemic conditions, symptoms or clinical biomarkers of disease are reduced. Alternatively, treatment is “effective” if the progression of a disease is reduced or halted. That is, “treatment” includes not just the improvement of symptoms or biomarkers, but also a cessation of, or at least slowing of, progress or worsening of symptoms compared to what would be expected in the absence of treatment. Thus, a treatment is considered effective if one or more of the signs or symptoms of a condition described herein are altered in a beneficial manner, other clinically accepted symptoms are improved, or even ameliorated and/or reversed back to a more normal or normal state, or a desired response is induced e.g., by at least 10% following treatment according to the methods described herein. Beneficial or desired clinical results include, but are not limited to, alleviation of one or more symptom(s), diminishment of extent of disease, e.g., chronic inflammatory disease, stabilized (e.g., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the terms “ameliorate,” “ameliorating,” “amelioration,” or “ameliorated” in reference to cancer can mean reducing the symptoms of the cancer, reducing the size of a tumor, completely or partially removing the tumor (e.g., a complete or partial response), causing stable disease, preventing progression of the cancer (e.g., progression free survival), or any other effect on the cancer that would be considered by a physician to be a therapeutic, prophylactic, or preventative treatment of the cancer.

As used herein, the terms “administer,” administering,” or “administered” mean all means of introducing the compound, or pharmaceutically acceptable salt thereof, or CAR T-cell composition, where the CAR T-cell composition includes CAR T-cells and where the CAR includes an E2 anti-fluorescein antibody fragment, to the patient, including, but not limited to, oral, intravenous, intramuscular, subcutaneous, and transdermal. The term “effective amount” as used herein refers to the amount of an active agent or composition needed to alleviate at least one or more criteria of the disease or disorder, and relates to a sufficient amount of active agent or pharmacological composition to provide the desired effect. The term “therapeutically effective amount” therefore refers to an amount of active agent or composition that is sufficient to provide a particular anti-bacterial or anti-recolonization effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “transduction” and “transfection” are used equivalently and the terms mean introducing a nucleic acid into a cell by any artificial method, including viral and non-viral methods.

The term “in vitro” as used herein has its ordinary meaning as understood in light of the specification, and refers to a system or condition in a cell, tissue, or organ outside of a subject's body. In some embodiments, the cell, tissue, or organ is not a primary cell, tissue, or organ taken directly from the subject. In some embodiments, the cell is an established cell line. In some embodiments, the cell is derived from a primary cell.

The term “ex vivo” as used herein has its ordinary meaning as understood in light of the specification, and refers to a system or condition in a cell, tissue, or organ outside of a subject's body, which is later returned to the subject's body.

The term “in vivo” as used herein has its ordinary meaning as understood in light of the specification, and refers to a system or condition within a subject's body.

The terms “primary cell,” “primary tissue,” and “primary organ” have their ordinary meaning as understood in light of the specification, and refer to a cell, tissue, or organ, respectively, that has been directly taken from a subject.

The term “comprising” as used herein is synonymous with “including,” “containing,” or “characterized by,” and is inclusive or open-ended and does not exclude additional, unrecited elements or method steps.

The above description discloses several methods and materials. This disclosure is susceptible to modifications in the methods and materials, as well as alterations in the fabrication methods and equipment. Such modifications will become apparent to those skilled in the art from a consideration of the disclosure disclosed herein. Consequently, it is not intended that embodiments described herein be limited to the specific embodiments disclosed herein, but that it covers all modifications and alternatives coming within the true scope and spirit of the disclosure.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

In another embodiment of the methods described herein, any of the methods described herein can be used alone, or any of the methods described herein can be used in combination with any other method or methods described herein.

Some embodiments provided herein are described by way of the following provided numbered alternatives.
1. A modified IL-12 protein construct comprising a p35 subdomain, a linker, and a p40 subdomain.
2. The modified IL-12 protein construct of alternative 1, wherein the linker is 4, 5, 6, 7, 8, 9, 10, 11, 12, 13, 14, or 15 amino acids in length.
3. The modified IL-12 protein construct of any one of alternatives 1-2, wherein the p35 subdomain is on the C terminus, and the p40 subdomain is on the N terminus.
4. The modified IL-12 protein construct of any one of alternatives 1-2, wherein the p35 subdomain is on the N terminus, and the p40 subdomain is on the C terminus.
5. The modified IL-12 protein construct of any one of alternatives 1-4, wherein the linker comprises a sequence with at least 80% identity to any one of the sequences of SEQ ID NOs: 23-26.
6. The modified IL-12 protein construct of any one of alternatives 1-5, wherein the p40 subdomain comprises a sequence with at least 80% identity to any one of the sequences of SEQ ID NOs: 19 or 20.
7. The modified IL-12 protein construct of alternative 6, wherein the p40 subdomain further comprises one, two, three, or four mutations.
8. The modified IL-12 protein construct of alternative 7, wherein the mutations comprise mutations of E81A, F82A, K106A, or K219A.
9. The modified IL-12 protein construct of any one of alternatives 1-8, wherein the p35 subdomain comprises a sequence with at least 80% identity to any one of the sequences of SEQ ID NOs: 21 or 22.
10. A modified IL-12 protein construct, wherein the construct comprises a sequence with at least 80% identity to any one of the sequences of SEQ ID NOs: 1-9.
11. The modified IL-12 protein construct of alternative 10, wherein a p40 subdomain of the IL-12 protein construct further comprises one, two, three, or four mutations.
12. The modified IL-12 protein construct of alternative 11, wherein the mutations comprise mutations of E81A, F82A, K106A, or K219A.
13. A nucleotide sequence encoding any one of the modified IL-12 protein constructs of alternatives 1-12.
14. A nucleotide sequence expressing a modified IL-12 protein construct, wherein the nucleotide sequence comprises a sequence with at least 80% identity to any one of the sequences of SEQ ID NOs: 10-18.
15. A vector encoding any one of the modified IL-12 protein constructs of alternatives 1-9, and/or any one of the nucleotide sequences of alternatives 13-14.
16. A cell containing and/or secreting any one of the modified IL-12 protein constructs of alternatives 1-12, any one of the nucleotide sequences of alternatives 13-14, and/or the vector of alternative 15, wherein the cell expresses the modified IL-12 protein.
17. The cell of alternative 16, wherein the cell is a lymphocyte and/or leukocyte.
18. The cell of alternative 17, wherein the cell is a T lymphocyte or a cytotoxic T lymphocyte.
19. The cell of any one of alternatives 16-18, wherein the modified IL-12 protein has a reduced affinity for IL-12 receptors compared to wild type IL-12.
20. The cell of any one of alternatives 16-18, wherein the modified IL-12 protein has a reduced stability and/or half-life compared to wild type IL-12.
21. The cell of any one of alternatives 16-20, wherein the modified IL-12 protein has a reduced expression compared to wild type IL-12.
22. The cell of alternative 21, wherein the reduced expression comprises reduced concentration of IL-12 in a cell, a tissue, an organ, a blood sample, a plasma sample, a system, and/or a subject.
23. The cell of any one of alternatives 16-22, wherein the cell leads to decreased level of IFNγ production by effector cells when compared to a cell expressing wild type IL-12.
24. A method of treating cancer in a subject, the method comprising administering to the subject the cell of any one of alternatives 16-23.
25. The method of alternative 24, wherein the subject is mammalian and/or human.
26. The method of any one of alternatives 24-25, wherein the cancer is pancreatic cancer, prostate cancer, breast cancer, ovarian cancer, colorectal cancer, lung cancer, renal cell carcinoma, bladder cancer, medulloblastoma or neuroblastoma.
27. A method of treating cancer in a subject, the method comprising administering to the subject a modified IL-12 protein construct comprising a p35 subdomain, a linker, and a p40 subdomain.
28. The method of alternative 27, wherein the linker is 4, 5, 6, 7, 8, 9, 10, 11, 12, 13, 14, or 15 amino acids in length.
29. The method of any one of alternatives 27-28, wherein the p35 subdomain is on the C terminus, and the p40 subdomain is on the N terminus.
30. The method of any one of alternatives 27-28, wherein the p35 subdomain is on the N terminus, and the p40 subdomain is on the C terminus.
31. The method of any one of alternatives 27-30, wherein the linker comprises a sequence with at least 80% identity to any one of the sequences of SEQ ID NOs: 23-26.
32. The method of any one of alternatives 27-31, wherein the p40 subdomain comprises a sequence with at least 80% identity to any one of the sequences of SEQ ID NOs: 19 or 20.
33. The method of alternative 32, wherein the p40 subdomain further comprises one, two, three, or four mutations.
34. The method of alternative 33, wherein the mutations comprise mutations of E81A, F82A, K106A, or K219A.
35. The method of any one of alternatives 27-34, wherein the p35 subdomain comprises a sequence with at least 80% identity to any one of the sequences of SEQ ID NOs: 21 or 22.
36. The method of any one of alternatives 27-35, wherein the subject is mammalian and/or human.
37. The method of any one of alternatives 27-36, wherein the cancer is pancreatic cancer, prostate cancer, breast cancer, ovarian cancer, colorectal cancer, lung cancer, renal cell carcinoma, bladder cancer, medulloblastoma or neuroblastoma.
38. A composition comprising a modified IL-12 protein construct, wherein the modified IL-12 protein construct comprises a p35 subdomain, a linker, and a p40 subdomain.
39. The composition of alternative 38, wherein the linker is 4, 5, 6, 7, 8, 9, 10, 11, 12, 13, 14, or 15 amino acids in length.
40. The composition of any one of alternatives 38-39, wherein the p35 subdomain is on the C terminus, and the p40 subdomain is on the N terminus.
41. The composition of any one of alternatives 38-39, wherein the p35 subdomain is on the N terminus, and the p40 subdomain is on the C terminus.
42. The composition of any one of alternatives 38-41, wherein the linker comprises a sequence with at least 80% identity to any one of the sequences of SEQ ID NOs: 23-26.
43. The composition of any one of alternatives 38-42, wherein the p40 subdomain comprises a sequence with at least 80% identity to any one of the sequences of SEQ ID NOs: 19 or 20.
44. The composition of alternative 43, wherein the p40 subdomain further comprises one, two, three, or four mutations.
45. The composition of alternative 44, wherein the mutations comprise mutations of E81A, F82A, K106A, or K219A.
46. The composition of any one of alternatives 38-45, wherein the p35 subdomain comprises a sequence with at least 80% identity to any one of the sequences of SEQ ID NOs: 21 or 22.
47. The composition of any one of alternatives 38-46, wherein the composition further comprises a cell.
48. The composition of alternative 47, wherein the cell is a T lymphocyte or a cytotoxic T lymphocyte.
49. A pharmaceutical composition comprising an effective carrier and a modified IL-12 protein construct, wherein the modified IL-12 protein construct comprises a p35 subdomain, a linker, and a p40 subdomain.
50. The pharmaceutical composition of alternative 49, wherein the linker is 4, 5, 6, 7, 8, 9, 10, 11, 12, 13, 14, or 15 amino acids in length.
51. The pharmaceutical composition of any one of alternatives 49-50, wherein the p35 subdomain is on the C terminus, and the p40 subdomain is on the N terminus.
52. The pharmaceutical composition of any one of alternatives 49-50, wherein the p35 subdomain is on the N terminus, and the p40 subdomain is on the C terminus.
53. The pharmaceutical composition of any one of alternatives 49-52, wherein the linker comprises a sequence with at least 80% identity to any one of the sequences of SEQ ID NOs: 23-26.
54. The pharmaceutical composition of any one of alternatives 49-43, wherein the p40 subdomain comprises a sequence with at least 80% identity to any one of the sequences of SEQ ID NOs: 19 or 20.
55. The pharmaceutical composition of alternative 54, wherein the p40 subdomain further comprises one, two, three, or four mutations.
56. The pharmaceutical composition of alternative 55, wherein the mutations comprise mutations of E81A, F82A, K106A, or K219A.
57. The pharmaceutical composition of any one of alternatives 49-56, wherein the p35 subdomain comprises a sequence with at least 80% identity to any one of the sequences of SEQ ID NOs: 21 or 22.
58. The pharmaceutical composition of any one of alternatives 49-57, wherein the composition further comprises a cell.
59. The pharmaceutical composition of alternative 58, wherein the cell is a T lymphocyte or a cytotoxic T lymphocyte.
60. A method of treating a disease or disorder in a subject, the method comprising administering to the subject a modified IL-12 protein construct comprising a p35 subdomain, a linker, and a p40 subdomain.
61. The method of alternative 60, wherein the linker is 4, 5, 6, 7, 8, 9, 10, 11, 12, 13, 14, or 15 amino acids in length.
62. A method of treating cancer in a subject, the method comprising administering to the subject a cell expressing a modified IL-12 protein construct comprising a p35 subdomain, a linker, and a p40 subdomain.
63. The method of alternative 62, wherein the linker is 4, 5, 6, 7, 8, 9, 10, 11, 12, 13, 14, or 15 amino acids in length.
64. The method of any one of alternatives 62-63, wherein the cell is a lymphocyte and/or leukocyte.
65. The method of any one of alternatives 62-64, wherein the cell is a T lymphocyte or a cytotoxic T lymphocyte.
66. A composition comprising a modified IL-12 protein construct for use in the treatment of cancer, wherein the IL-12 protein construct comprises a p35 subdomain, a linker, and a p40 subdomain.
67. The composition for use of alternative 66, wherein the linker is 4, 5, 6, 7, 8, 9, 10, 11, 12, 13, 14, or 15 amino acids in length.
68. A cell expressing a modified IL-12 protein construct for use in the treatment of cancer, wherein the IL-12 protein construct comprises a p35 subdomain, a linker, and a p40 subdomain.
69. The cell for use of alternative 68, wherein the linker is 4, 5, 6, 7, 8, 9, 10, 11, 12, 13, 14, or 15 amino acids in length.
70. The cell for use of any one of alternatives 68-69, wherein the cell is a lymphocyte and/or leukocyte.
71. The cell for use of any one of alternatives 68-70, wherein the cell is a T lymphocyte or a cytotoxic T lymphocyte.

### EXAMPLES

While the present disclosure has been described in some detail for purposes of clarity and understanding, one skilled in the art will appreciate that various changes in form and detail can be made without departing from the true scope of the present disclosure.

### Example 1: Modulation of IL-12 Signaling Through Optimization of Linker in p35-p40 Orientation

Single chain versions of IL-12 (scIL-12) can be produced that enable the molecule to be placed in a single transgene and expressed (for example) from a retroviral vector that encodes a CAR. A series of modified scIL-12 protein constructs were generated with alternating lengths, sequences and orientations. The generated constructs are as summarized in the below Table 1, and specific sequences are as shown in Tables 2-5.

TABLE 1
IL-12 Protein Constructs

| | SEQ | |
|---|---|---|
| Construct: | ID NO: | Domains: |
| CM065 | 1 | p40 - 15aa linker - p35 |
| CM066 | 2 | p40 - 7aa linker - p35 |
| CM063 | 3 | p35 - 15aa linker - p40 |
| CM121 | 4 | p35 - 10aa linker - p40 |
| CM064 | 5 | p35 - 7aa linker - p40 |
| CM120 | 6 | p35 - 5aa linker - p40 |
| CM067 | 7 | Δp40 (E81A, F82A) - 15aa linker - p35 |
| CM068 | 8 | Δp40 (E81A, F82A, K106A) - 15aa linker - p35 |
| CM069 | 9 | Δp40 (E81A, F82A, K106A, K219A) - 15 aa |
| | | linker - p35 |

TABLE 2
Amino Acid Sequences of scIL-12 Variants

| SEQ ID | | |
|---|---|---|
| NO: | Description | Sequence |
| 1 | p40-15 aa | MCHQQLVISWFSLVFLASPLVAIWELKKDVYVVELDWYPDAPGEMVVLT |
| | linker-p35 | CDTPEEDGITWTLDQSSEVLGSGKTLTIQVKEFGDAGQYTCHKGGEVLSH |
| | | SLLLLHKKEDGIWSTDILKDQKEPKNKTFLRCEAKNYSGRFTCWWLTTIS |
| | | TDLTFSVKSSRGSSDPQGVTCGAATLSAERVRGDNKEYEYSVECQEDSAC |
| | | PAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICR |
| | | KNASISVRAQDRYYSSSWSEWASVPCSGGGGSGGGGSGGGGSRNLPVAT |
| | | PDPGMFPCLHHSQNLLRAVSNMLQKARQTLEFYPCTSEEIDHEDITKDKTS |
| | | TVEACLPLELTKNESCLNSRETSFITNGSCLASRKTSFMMALCLSSIYEDLK |
| | | MYQVEFKTMNAKLLMDPKRQIFLDQNMLAVIDELMQALNFNSETVPQKS |
| | | SLEEPDFYKTKIKLCILLHAFRIRAVTIDRVMSYLNAS |
| 2 | p40-7 aa | MCHQQLVISWFSLVFLASPLVAIWELKKDVYVVELDWYPDAPGEMVVLT |
| | linker-p35 | CDTPEEDGITWTLDQSSEVLGSGKTLTIQVKEFGDAGQYTCHKGGEVLSH |
| | | SLLLLHKKEDGIWSTDILKDQKEPKNKTFLRCEAKNYSGRFTCWWLTTIS |
| | | TDLTFSVKSSRGSSDPQGVTCGAATLSAERVRGDNKEYEYSVECQEDSAC |
| | | PAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICR |
| | | KNASISVRAQDRYYSSSWSEWASVPCSGGGGGGSRNLPVATPDPGMFPC |
| | | LHHSQNLLRAVSNMLQKARQTLEFYPCTSEEIDHEDITKDKTSTVEACLPL |
| | | ELTKNESCLNSRETSFITNGSCLASRKTSFMMALCLSSIYEDLKMYQVEFK |
| | | TMNAKLLMDPKRQIFLDQNMLAVIDELMQALNFNSETVPQKSSLEEPDFY |
| | | KTKIKLCILLHAFRIRAVTIDRVMSYLNAS |
| 3 | p35-15 aa | MCPARSLLLVATLVLLDHLSLARNLPVATPDPGMFPCLHHSQNLLRAVSN |
| | linker-p40 | MLQKARQTLEFYPCTSEEIDHEDITKDKTSTVEACLPLELTKNESCLNSRE |
| | | TSFITNGSCLASRKTSFMMALCLSSIYEDLKMYQVEFKTMNAKLLMDPKR |
| | | QIFLDQNMLAVIDELMQALNFNSETVPQKSSLEEPDFYKTKIKLCILLHAF |
| | | RIRAVTIDRVMSYLNASGGGGSGGGGSGGGGSIWELKKDVYVVELDWYP |
| | | DAPGEMVVLTCDTPEEDGITWTLDQSSEVLGSGKTLTIQVKEFGDAGQYT |
| | | CHKGGEVLSHSLLLLHKKEDGIWSTDILKDQKEPKNKTFLRCEAKNYSGR |
| | | FTCWWLTTISTDLTFSVKSSRGSSDPQGVTCGAATLSAERVRGDNKEYEY |
| | | SVECQEDSACPAAEESLPIEVMVDAVHKLKYENYTSSFFIRDIIKPDPPKNL |
| | | QLKPLKNSRQVEVSWEYPDTWSTPHSYFSLTFCVQVQGKSKREKKDRVF |
| | | TDKTSATVICRKNASISVRAQDRYYSSSWSEWASVPCS |
| 4 | p35-10 aa | MCPARSLLLVATLVLLDHLSLARNLPVATPDPGMFPCLHHSQNLLRAVSN |
| | linker-p40 | MLQKARQTLEFYPCTSEEIDHEDITKDKTSTVEACLPLELTKNESCLNSRE |
| | | TSFITNGSCLASRKTSFMMALCLSSIYEDLKMYQVEFKTMNAKLLMDPKR |
| | | QIFLDQNMLAVIDELMQALNFNSETVPQKSSLEEPDFYKTKIKLCILLHAF |
| | | RIRAVTIDRVMSYLNASGGGGSGGGGSIWELKKDVYVVELDWYPDAPGE |
| | | MVVLTCDTPEEDGITWTLDQSSEVLGSGKTLTIQVKEFGDAGQYTCHKG |
| | | GEVLSHSLLLLHKKEDGIWSTDILKDQKEPKNKTFLRCEAKNYSGRFTCW |
| | | WLTTISTDLTFSVKSSRGSSDPQGVTCGAATLSAERVRGDNKEYEYSVEC |
| | | QEDSACPAAEESLPIEVMVDAVHKLKYENYTSSFFIRDIIKPDPPKNLQLKP |
| | | LKNSRQVEVSWEYPDTWSTPHSYFSLTFCVQVQGKSKREKKDRVFTDKT |
| | | SATVICRKNASISVRAQDRYYSSSWSEWASVPCS |
| 5 | p35-7 aa | MCPARSLLLVATLVLLDHLSLARNLPVATPDPGMFPCLHHSQNLLRAVSN |
| | linker-p40 | MLQKARQTLEFYPCTSEEIDHEDITKDKTSTVEACLPLELTKNESCLNSRE |
| | | TSFITNGSCLASRKTSFMMALCLSSIYEDLKMYQVEFKTMNAKLLMDPKR |
| | | QIFLDQNMLAVIDELMQALNFNSETVPQKSSLEEPDFYKTKIKLCILLHAF |
| | | RIRAVTIDRVMSYLNASGGGGGGSIWELKKDVYVVELDWYPDAPGEMV |
| | | VLTCDTPEEDGITWTLDQSSEVLGSGKTLTIQVKEFGDAGQYTCHKGGEV |
| | | LSHSLLLLHKKEDGIWSTDILKDQKEPKNKTFLRCEAKNYSGRFTCWWLT |
| | | TISTDLTFSVKSSRGSSDPQGVTCGAATLSAERVRGDNKEYEYSVECQEDS |
| | | ACPAAEESLPIEVMVDAVHKLKYENYTSSFFIRDIIKPDPPKNLQLKPLKNS |
| | | RQVEVSWEYPDTWSTPHSYFSLTFCVQVQGKSKREKKDRVFTDKTSATVI |
| | | CRKNASISVRAQDRYYSSSWSEWASVPCS |
| 6 | p35-5 aa | MCPARSLLLVATLVLLDHLSLARNLPVATPDPGMFPCLHHSQNLLRAVSN |
| | linker-p40 | MLQKARQTLEFYPCTSEEIDHEDITKDKTSTVEACLPLELTKNESCLNSRE |
| | | TSFITNGSCLASRKTSFMMALCLSSIYEDLKMYQVEFKTMNAKLLMDPKR |
| | | QIFLDQNMLAVIDELMQALNFNSETVPQKSSLEEPDFYKTKIKLCILLHAF |
| | | RIRAVTIDRVMSYLNASGGGGSIWELKKDVYVVELDWYPDAPGEMVVLT |
| | | CDTPEEDGITWTLDQSSEVLGSGKTLTIQVKEFGDAGQYTCHKGGEVLSH |
| | | SLLLLHKKEDGIWSTDILKDQKEPKNKTFLRCEAKNYSGRFTCWWLTTIS |
| | | TDLTFSVKSSRGSSDPQGVTCGAATLSAERVRGDNKEYEYSVECQEDSAC |
| | | PAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICR |
| | | KNASISVRAQDRYYSSSWSEWASVPCS |
| 7 | p40 (E81A, | MCHQQLVISWFSLVFLASPLVAIWELKKDVYVVELDWYPDAPGEMVVLT |
| | F82A)-15 aa | CDTPEEDGITWTLDQSSEVLGSGKTLTIQVKAAGDAGQYTCHKGGEVLSH |
| | linker-p35 | SLLLLHKKEDGIWSTDILKDQKEPKNKTFLRCEAKNYSGRFTCWWLTTIS |
| | | TDLTFSVKSSRGSSDPQGVTCGAATLSAERVRGDNKEYEYSVECQEDSAC |
| | | PAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICR |
| | | KNASISVRAQDRYYSSSWSEWASVPCSGGGGSGGGGSGGGGS |
| | | RNLPVATPDPGMFPCLHHSQNLLRAVSNMLQKARQTLEFYPCTSEEIDHE |
| | | DITKDKTSTVEACLPLELTKNESCLNSRETSFITNGSCLASRKTSFMMALC |
| | | LSSIYEDLKMYQVEFKTMNAKLLMDPKRQIFLDQNMLAVIDELMQALNF |
| | | NSETVPQKSSLEEPDFYKTKIKLCILLHAFRIRAVTIDRVMSYLNAS |
| 8 | p40 (E81A, | MCHQQLVISWFSLVFLASPLVAIWELKKDVYVVELDWYPDAPGEMVVLT |
| | F82A, | CDTPEEDGITWTLDQSSEVLGSGKTLTIQVKAAGDAGQYTCHKGGEVLSH |
| | K106A)- | SLLLLHAKEDGIWSTDILKDQKEPKNKTFLRCEAKNYSGRFTCWWLTTIS |
| | 15 aa linker- | TDLTFSVKSSRGSSDPQGVTCGAATLSAERVRGDNKEYEYSVECQEDSAC |
| | p35 | PAAEESLPIEVMVDAVHKLKYENYTSSFFIRDIIKPDPPKNLQLKPLKNSRQ |
| | | VEVSWEYPDTWSTPHSYFSLTFCVQVQGKSKREKKDRVFTDKTSATVICR |
| | | KNASISVRAQDRYYSSSWSEWASVPCSGGGGSGGGGSGGGGSRNLPVAT |
| | | PDPGMFPCLHHSQNLLRAVSNMLQKARQTLEFYPCTSEEIDHEDITKDKTS |
| | | TVEACLPLELTKNESCLNSRETSFITNGSCLASRKTSFMMALCLSSIYEDLK |
| | | MYQVEFKTMNAKLLMDPKRQIFLDQNMLAVIDELMQALNFNSETVPQKS |
| | | SLEEPDFYKTKIKLCILLHAFRIRAVTIDRVMSYLNAS |
| 9 | p40 (E81A, | MCHQQLVISWFSLVFLASPLVAIWELKKDVYVVELDWYPDAPGEMVVLT |
| | F82A, | CDTPEEDGITWTLDQSSEVLGSGKTLTIQVKAAGDAGQYTCHKGGEVLSH |
| | K106A, | SLLLLHAKEDGIWSTDILKDQKEPKNKTFLRCEAKNYSGRFTCWWLTTIS |
| | K219A)- | TDLTFSVKSSRGSSDPQGVTCGAATLSAERVRGDNKEYEYSVECQEDSAC |
| | 15 aa linker- | PAAEESLPIEVMVDAVHKLAYENYTSSFFIRDIIKPDPPKNLQLKPLKNSRQ |
| | p35 | VEVSWEYPDTWSTPHSYFSLTFCVQVQGKSKREKKDRVFTDKTSATVICR |
| | | KNASISVRAQDRYYSSSWSEWASVPCSGGGGSGGGGSGGGGSRNLPVAT |
| | | PDPGMFPCLHHSQNLLRAVSNMLQKARQTLEFYPCTSEEIDHEDITKDKTS |
| | | TVEACLPLELTKNESCLNSRETSFITNGSCLASRKTSFMMALCLSSIYEDLK |
| | | MYQVEFKTMNAKLLMDPKRQIFLDQNMLAVIDELMQALNENSETVPQKS |
| | | SLEEPDFYKTKIKLCILLHAFRIRAVTIDRVMSYLNAS |

TABLE 3
DNA Sequences of scIL-12 Variants

| SEQ ID | | |
|---|---|---|
| NO: | Description | Sequence |
| 10 | p40-15 aa | ATGTGTCACCAGCAGTTGGTCATCTCTTGGTTTTCCCTGGTTTTTCTGG |
| | linker-p35 | CATCTCCCCTCGTGGCCATATGGGAACTGAAGAAAGATGTTTATGTCG |
| | | TAGAATTGGATTGGTATCCGGATGCCCCTGGAGAAATGGTGGTCCTC |
| | | ACCTGTGACACCCCTGAAGAAGATGGTATCACCTGGACCTTGGACCA |
| | | GAGCAGTGAGGTCTTAGGCTCTGGCAAAACCCTGACCATCCAAGTCA |
| | | AAGAGTTTGGAGATGCTGGCCAGTACACCTGTCACAAAGGAGGCGAG |
| | | GTTCTAAGCCATTCGCTCCTGCTGCTTCACAAAAAGGAAGATGGAATT |
| | | TGGTCCACTGATATTTTAAAGGACCAGAAAGAACCCAAAAATAAGAC |
| | | CTTTCTAAGATGCGAGGCCAAGAATTATTCTGGACGTTTCACCTGCTG |
| | | GTGGCTGACGACAATCAGTACTGATTTGACATTCAGTGTCAAAAGCA |
| | | GCAGAGGCTCTTCTGACCCCCAAGGGGTGACGTGCGGAGCTGCTACA |
| | | CTCTCTGCAGAGAGAGTCAGAGGGGACAACAAGGAGTATGAGTACTC |
| | | AGTGGAGTGCCAGGAGGACAGTGCCTGCCCAGCTGCTGAGGAGAGTC |
| | | TGCCCATTGAGGTCATGGTGGATGCCGTTCACAAGCTCAAGTATGAA |
| | | AACTACACCAGCAGCTTCTTCATCAGGGACATCATCAAACCTGACCC |
| | | ACCCAAGAACTTGCAGCTGAAGCCATTAAAGAATTCTCGGCAGGTGG |
| | | AGGTCAGCTGGGAGTACCCTGACACCTGGAGTACTCCACATTCCTACT |
| | | TCTCCCTGACATTCTGCGTTCAGGTCCAGGGCAAGAGCAAGAGAGAA |
| | | AAGAAAGATAGAGTCTTCACGGACAAGACCTCAGCCACGGTCATCTG |
| | | CCGCAAAAATGCCAGCATTAGCGTGCGGGCCCAGGACCGCTACTATA |
| | | GCTCATCTTGGAGCGAATGGGCATCTGTGCCCTGCAGT GGT GGC GGT |
| | | GGATCTGGCGGTGGCGGTTCTGGCGGTGGAGGCTCTAGAAACCTCCC |
| | | CGTGGCCACTCCAGACCCAGGAATGTTCCCATGCCTTCACCACTCCCA |
| | | AAACCTGCTGAGGGCCGTCAGCAACATGCTCCAGAAGGCCAGACAAA |
| | | CTCTAGAATTTTACCCTTGCACTTCTGAAGAGATTGATCATGAAGATA |
| | | TCACAAAAGATAAAACCAGCACAGTGGAGGCCTGTTTACCATTGGAA |
| | | TTAACCAAGAATGAGAGTTGCCTAAATTCCAGAGAGACCTCTTTCATA |
| | | ACTAATGGGAGTTGCCTGGCCTCCAGAAAGACCTCTTTTATGATGGCC |
| | | CTGTGCCTTAGTAGTATTTATGAAGACTTGAAGATGTACCAGGTGGAG |
| | | TTCAAGACCATGAATGCAAAGCTTCTGATGGATCCTAAGAGGCAGAT |
| | | CTTTCTAGATCAAAACATGCTGGCAGTTATTGATGAGCTGATGCAGGC |
| | | CCTGAATTTCAACAGTGAGACTGTGCCACAAAAATCCTCCCTTGAAG |
| | | AACCGGATTTTTATAAAACTAAAATCAAGCTCTGCATACTTCTTCATG |
| | | CTTTCAGAATTCGGGCAGTGACTATTGATAGAGTGATGAGCTATCTGA |
| | | ATGCTTCCTGA |
| 11 | p40-7 aa | ATGTGTCACCAGCAGTTGGTCATCTCTTGGTTTTCCCTGGTTTTTCTGG |
| | linker-p35 | CATCTCCCCTCGTGGCCATATGGGAACTGAAGAAAGATGTTTATGTCG |
| | | TAGAATTGGATTGGTATCCGGATGCCCCTGGAGAAATGGTGGTCCTC |
| | | ACCTGTGACACCCCTGAAGAAGATGGTATCACCTGGACCTTGGACCA |
| | | GAGCAGTGAGGTCTTAGGCTCTGGCAAAACCCTGACCATCCAAGTCA |
| | | AAGAGTTTGGAGATGCTGGCCAGTACACCTGTCACAAAGGAGGCGAG |
| | | GTTCTAAGCCATTCGCTCCTGCTGCTTCACAAAAAGGAAGATGGAATT |
| | | TGGTCCACTGATATTTTAAAGGACCAGAAAGAACCCAAAAATAAGAC |
| | | CTTTCTAAGATGCGAGGCCAAGAATTATTCTGGACGTTTCACCTGCTG |
| | | GTGGCTGACGACAATCAGTACTGATTTGACATTCAGTGTCAAAAGCA |
| | | GCAGAGGCTCTTCTGACCCCCAAGGGGTGACGTGCGGAGCTGCTACA |
| | | CTCTCTGCAGAGAGAGTCAGAGGGGACAACAAGGAGTATGAGTACTC |
| | | AGTGGAGTGCCAGGAGGACAGTGCCTGCCCAGCTGCTGAGGAGAGTC |
| | | TGCCCATTGAGGTCATGGTGGATGCCGTTCACAAGCTCAAGTATGAA |
| | | AACTACACCAGCAGCTTCTTCATCAGGGACATCATCAAACCTGACCC |
| | | ACCCAAGAACTTGCAGCTGAAGCCATTAAAGAATTCTCGGCAGGTGG |
| | | AGGTCAGCTGGGAGTACCCTGACACCTGGAGTACTCCACATTCCTACT |
| | | TCTCCCTGACATTCTGCGTTCAGGTCCAGGGCAAGAGCAAGAGAGAA |
| | | AAGAAAGATAGAGTCTTCACGGACAAGACCTCAGCCACGGTCATCTG |
| | | CCGCAAAAATGCCAGCATTAGCGTGCGGGCCCAGGACCGCTACTATA |
| | | GCTCATCTTGGAGCGAATGGGCATCTGTGCCCTGCAGTGGCGGAGGT |
| | | GGAGGCGGTTCTAGAAACCTCCCCGTGGCCACTCCAGACCCAGGAAT |
| | | GTTCCCATGCCTTCACCACTCCCAAAACCTGCTGAGGGCCGTCAGCAA |
| | | CATGCTCCAGAAGGCCAGACAAACTCTAGAATTTTACCCTTGCACTTC |
| | | TGAAGAGATTGATCATGAAGATATCACAAAAGATAAAACCAGCACAG |
| | | TGGAGGCCTGTTTACCATTGGAATTAACCAAGAATGAGAGTTGCCTA |
| | | AATTCCAGAGAGACCTCTTTCATAACTAATGGGAGTTGCCTGGCCTCC |
| | | AGAAAGACCTCTTTTATGATGGCCCTGTGCCTTAGTAGTATTTATGAA |
| | | GACTTGAAGATGTACCAGGTGGAGTTCAAGACCATGAATGCAAAGCT |
| | | TCTGATGGATCCTAAGAGGCAGATCTTTCTAGATCAAAACATGCTGGC |
| | | AGTTATTGATGAGCTGATGCAGGCCCTGAATTTCAACAGTGAGACTGT |
| | | GCCACAAAAATCCTCCCTTGAAGAACCGGATTTTTATAAAACTAAAA |
| | | TCAAGCTCTGCATACTTCTTCATGCTTTCAGAATTCGGGCAGTGACTA |
| | | TTGATAGAGTGATGAGCTATCTGAATGCTTCCTGA |
| 12 | p35-15 aa | ATGTGTCCAGCGCGCAGCCTCCTCCTTGTGGCTACCCTGGTCCTCCTG |
| | linker-p40 | GACCACCTCAGTTTGGCCAGAAACCTCCCCGTGGCCACTCCAGACCC |
| | | AGGAATGTTCCCATGCCTTCACCACTCCCAAAACCTGCTGAGGGCCGT |
| | | CAGCAACATGCTCCAGAAGGCCAGACAAACTCTAGAATTTTACCCTT |
| | | GCACTTCTGAAGAGATTGATCATGAAGATATCACAAAAGATAAAACC |
| | | AGCACAGTGGAGGCCTGTTTACCATTGGAATTAACCAAGAATGAGAG |
| | | TTGCCTAAATTCCAGAGAGACCTCTTTCATAACTAATGGGAGTTGCCT |
| | | GGCCTCCAGAAAGACCTCTTTTATGATGGCCCTGTGCCTTAGTAGTAT |
| | | TTATGAAGACTTGAAGATGTACCAGGTGGAGTTCAAGACCATGAATG |
| | | CAAAGCTTCTGATGGATCCTAAGAGGCAGATCTTTCTAGATCAAAAC |
| | | ATGCTGGCAGTTATTGATGAGCTGATGCAGGCCCTGAATTTCAACAGT |
| | | GAGACTGTGCCACAAAAATCCTCCCTTGAAGAACCGGATTTTTATAA |
| | | AACTAAAATCAAGCTCTGCATACTTCTTCATGCTTTCAGAATTCGGGC |
| | | AGTGACTATTGATAGAGTGATGAGCTATCTGAATGCTTCCGGTGGC |
| | | GGTGGATCTGGCGGTGGCGGTTCTGGCGGTGGAGGCTCTATATGGGA |
| | | ACTGAAGAAAGATGTTTATGTCGTAGAATTGGATTGGTATCCGGATG |
| | | CCCCTGGAGAAATGGTGGTCCTCACCTGTGACACCCCTGAAGAAGAT |
| | | GGTATCACCTGGACCTTGGACCAGAGCAGTGAGGTCTTAGGCTCTGG |
| | | CAAAACCCTGACCATCCAAGTCAAAGAGTTTGGAGATGCTGGCCAGT |
| | | ACACCTGTCACAAAGGAGGCGAGGTTCTAAGCCATTCGCTCCTGCTG |
| | | CTTCACAAAAAGGAAGATGGAATTTGGTCCACTGATATTTTAAAGGA |
| | | CCAGAAAGAACCCAAAAATAAGACCTTTCTAAGATGCGAGGCCAAGA |
| | | ATTATTCTGGACGTTTCACCTGCTGGTGGCTGACGACAATCAGTACTG |
| | | ATTTGACATTCAGTGTCAAAAGCAGCAGAGGCTCTTCTGACCCCCAA |
| | | GGGGTGACGTGCGGAGCTGCTACACTCTCTGCAGAGAGAGTCAGAGG |
| | | GGACAACAAGGAGTATGAGTACTCAGTGGAGTGCCAGGAGGACAGT |
| | | GCCTGCCCAGCTGCTGAGGAGAGTCTGCCCATTGAGGTCATGGTGGA |
| | | TGCCGTTCACAAGCTCAAGTATGAAAACTACACCAGCAGCTTCTTCAT |
| | | CAGGGACATCATCAAACCTGACCCACCCAAGAACTTGCAGCTGAAGC |
| | | CATTAAAGAATTCTCGGCAGGTGGAGGTCAGCTGGGAGTACCCTGAC |
| | | ACCTGGAGTACTCCACATTCCTACTTCTCCCTGACATTCTGCGTTCAG |
| | | GTCCAGGGCAAGAGCAAGAGAGAAAAGAAAGATAGAGTCTTCACGG |
| | | ACAAGACCTCAGCCACGGTCATCTGCCGCAAAAATGCCAGCATTAGC |
| | | GTGCGGGCCCAGGACCGCTACTATAGCTCATCTTGGAGCGAATGGGC |
| | | ATCTGTGCCCTGCAGTTGA |
| 13 | p35-10 aa | ATGTGTCCAGCGCGCAGCCTCCTCCTTGTGGCTACCCTGGTCCTCCTG |
| | linker-p40 | GACCACCTCAGTTTGGCCAGAAACCTCCCCGTGGCCACTCCAGACCC |
| | | AGGAATGTTCCCATGCCTTCACCACTCCCAAAACCTGCTGAGGGCCGT |
| | | CAGCAACATGCTCCAGAAGGCCAGACAAACTCTAGAATTTTACCCTT |
| | | GCACTTCTGAAGAGATTGATCATGAAGATATCACAAAAGATAAAACC |
| | | AGCACAGTGGAGGCCTGTTTACCATTGGAATTAACCAAGAATGAGAG |
| | | TTGCCTAAATTCCAGAGAGACCTCTTTCATAACTAATGGGAGTTGCCT |
| | | GGCCTCCAGAAAGACCTCTTTTATGATGGCCCTGTGCCTTAGTAGTAT |
| | | TTATGAAGACTTGAAGATGTACCAGGTGGAGTTCAAGACCATGAATG |
| | | CAAAGCTTCTGATGGATCCTAAGAGGCAGATCTTTCTAGATCAAAAC |
| | | ATGCTGGCAGTTATTGATGAGCTGATGCAGGCCCTGAATTTCAACAGT |
| | | GAGACTGTGCCACAAAAATCCTCCCTTGAAGAACCGGATTTTTATAA |
| | | AACTAAAATCAAGCTCTGCATACTTCTTCATGCTTTCAGAATTCGGGC |
| | | AGTGACTATTGATAGAGTGATGAGCTATCTGAATGCTTCCGGTGGC |
| | | GGTGGATCTGGCGGTGGCGGTTCTATATGGGAACTGAAGAAAGATGT |
| | | TTATGTCGTAGAATTGGATTGGTATCCGGATGCCCCTGGAGAAATGGT |
| | | GGTCCTCACCTGTGACACCCCTGAAGAAGATGGTATCACCTGGACCTT |
| | | GGACCAGAGCAGTGAGGTCTTAGGCTCTGGCAAAACCCTGACCATCC |
| | | AAGTCAAAGAGTTTGGAGATGCTGGCCAGTACACCTGTCACAAAGGA |
| | | GGCGAGGTTCTAAGCCATTCGCTCCTGCTGCTTCACAAAAAGGAAGA |
| | | TGGAATTTGGTCCACTGATATTTTAAAGGACCAGAAAGAACCCAAAA |
| | | ATAAGACCTTTCTAAGATGCGAGGCCAAGAATTATTCTGGACGTTTCA |
| | | CCTGCTGGTGGCTGACGACAATCAGTACTGATTTGACATTCAGTGTCA |
| | | AAAGCAGCAGAGGCTCTTCTGACCCCCAAGGGGTGACGTGCGGAGCT |
| | | GCTACACTCTCTGCAGAGAGAGTCAGAGGGGACAACAAGGAGTATGA |
| | | GTACTCAGTGGAGTGCCAGGAGGACAGTGCCTGCCCAGCTGCTGAGG |
| | | AGAGTCTGCCCATTGAGGTCATGGTGGATGCCGTTCACAAGCTCAAG |
| | | TATGAAAACTACACCAGCAGCTTCTTCATCAGGGACATCATCAAACCT |
| | | GACCCACCCAAGAACTTGCAGCTGAAGCCATTAAAGAATTCTCGGCA |
| | | GGTGGAGGTCAGCTGGGAGTACCCTGACACCTGGAGTACTCCACATT |
| | | CCTACTTCTCCCTGACATTCTGCGTTCAGGTCCAGGGCAAGAGCAAGA |
| | | GAGAAAAGAAAGATAGAGTCTTCACGGACAAGACCTCAGCCACGGTC |
| | | ATCTGCCGCAAAAATGCCAGCATTAGCGTGCGGGCCCAGGACCGCTA |
| | | CTATAGCTCATCTTGGAGCGAATGGGCATCTGTGCCCTGCAGTTGA |
| 14 | p35-7 aa | ATGTGTCCAGCGCGCAGCCTCCTCCTTGTGGCTACCCTGGTCCTCCTG |
| | linker-p40 | GACCACCTCAGTTTGGCCAGAAACCTCCCCGTGGCCACTCCAGACCC |
| | | AGGAATGTTCCCATGCCTTCACCACTCCCAAAACCTGCTGAGGGCCGT |
| | | CAGCAACATGCTCCAGAAGGCCAGACAAACTCTAGAATTTTACCCTT |
| | | GCACTTCTGAAGAGATTGATCATGAAGATATCACAAAAGATAAAACC |
| | | AGCACAGTGGAGGCCTGTTTACCATTGGAATTAACCAAGAATGAGAG |
| | | TTGCCTAAATTCCAGAGAGACCTCTTTCATAACTAATGGGAGTTGCCT |
| | | GGCCTCCAGAAAGACCTCTTTTATGATGGCCCTGTGCCTTAGTAGTAT |
| | | TTATGAAGACTTGAAGATGTACCAGGTGGAGTTCAAGACCATGAATG |
| | | CAAAGCTTCTGATGGATCCTAAGAGGCAGATCTTTCTAGATCAAAAC |
| | | ATGCTGGCAGTTATTGATGAGCTGATGCAGGCCCTGAATTTCAACAGT |
| | | GAGACTGTGCCACAAAAATCCTCCCTTGAAGAACCGGATTTTTATAA |
| | | AACTAAAATCAAGCTCTGCATACTTCTTCATGCTTTCAGAATTCGGGC |
| | | AGTGACTATTGATAGAGTGATGAGCTATCTGAATGCTTCCGGCGGA |
| | | GGTGGAGGCGGTTCTATATGGGAACTGAAGAAAGATGTTTATGTCGT |
| | | AGAATTGGATTGGTATCCGGATGCCCCTGGAGAAATGGTGGTCCTCA |
| | | CCTGTGACACCCCTGAAGAAGATGGTATCACCTGGACCTTGGACCAG |
| | | AGCAGTGAGGTCTTAGGCTCTGGCAAAACCCTGACCATCCAAGTCAA |
| | | AGAGTTTGGAGATGCTGGCCAGTACACCTGTCACAAAGGAGGCGAGG |
| | | TTCTAAGCCATTCGCTCCTGCTGCTTCACAAAAAGGAAGATGGAATTT |
| | | GGTCCACTGATATTTTAAAGGACCAGAAAGAACCCAAAAATAAGACC |
| | | TTTCTAAGATGCGAGGCCAAGAATTATTCTGGACGTTTCACCTGCTGG |
| | | TGGCTGACGACAATCAGTACTGATTTGACATTCAGTGTCAAAAGCAG |
| | | CAGAGGCTCTTCTGACCCCCAAGGGGTGACGTGCGGAGCTGCTACAC |
| | | TCTCTGCAGAGAGAGTCAGAGGGGACAACAAGGAGTATGAGTACTCA |
| | | GTGGAGTGCCAGGAGGACAGTGCCTGCCCAGCTGCTGAGGAGAGTCT |
| | | GCCCATTGAGGTCATGGTGGATGCCGTTCACAAGCTCAAGTATGAAA |
| | | ACTACACCAGCAGCTTCTTCATCAGGGACATCATCAAACCTGACCCAC |
| | | CCAAGAACTTGCAGCTGAAGCCATTAAAGAATTCTCGGCAGGTGGAG |
| | | GTCAGCTGGGAGTACCCTGACACCTGGAGTACTCCACATTCCTACTTC |
| | | TCCCTGACATTCTGCGTTCAGGTCCAGGGCAAGAGCAAGAGAGAAAA |
| | | GAAAGATAGAGTCTTCACGGACAAGACCTCAGCCACGGTCATCTGCC |
| | | GCAAAAATGCCAGCATTAGCGTGCGGGCCCAGGACCGCTACTATAGC |
| | | TCATCTTGGAGCGAATGGGCATCTGTGCCCTGCAGTTGA |
| 15 | p35-5 aa | ATGTGTCCAGCGCGCAGCCTCCTCCTTGTGGCTACCCTGGTCCTCCTG |
| | linker-p40 | GACCACCTCAGTTTGGCCAGAAACCTCCCCGTGGCCACTCCAGACCC |
| | | AGGAATGTTCCCATGCCTTCACCACTCCCAAAACCTGCTGAGGGCCGT |
| | | CAGCAACATGCTCCAGAAGGCCAGACAAACTCTAGAATTTTACCCTT |
| | | GCACTTCTGAAGAGATTGATCATGAAGATATCACAAAAGATAAAACC |
| | | AGCACAGTGGAGGCCTGTTTACCATTGGAATTAACCAAGAATGAGAG |
| | | TTGCCTAAATTCCAGAGAGACCTCTTTCATAACTAATGGGAGTTGCCT |
| | | GGCCTCCAGAAAGACCTCTTTTATGATGGCCCTGTGCCTTAGTAGTAT |
| | | TTATGAAGACTTGAAGATGTACCAGGTGGAGTTCAAGACCATGAATG |
| | | CAAAGCTTCTGATGGATCCTAAGAGGCAGATCTTTCTAGATCAAAAC |
| | | ATGCTGGCAGTTATTGATGAGCTGATGCAGGCCCTGAATTTCAACAGT |
| | | GAGACTGTGCCACAAAAATCCTCCCTTGAAGAACCGGATTTTTATAA |
| | | AACTAAAATCAAGCTCTGCATACTTCTTCATGCTTTCAGAATTCGGGC |
| | | AGTGACTATTGATAGAGTGATGAGCTATCTGAATGCTTCCGGCGGA |
| | | GGTGGATCTATATGGGAACTGAAGAAAGATGTTTATGTCGTAGAATT |
| | | GGATTGGTATCCGGATGCCCCTGGAGAAATGGTGGTCCTCACCTGTG |
| | | ACACCCCTGAAGAAGATGGTATCACCTGGACCTTGGACCAGAGCAGT |
| | | GAGGTCTTAGGCTCTGGCAAAACCCTGACCATCCAAGTCAAAGAGTT |
| | | TGGAGATGCTGGCCAGTACACCTGTCACAAAGGAGGCGAGGTTCTAA |
| | | GCCATTCGCTCCTGCTGCTTCACAAAAAGGAAGATGGAATTTGGTCCA |
| | | CTGATATTTTAAAGGACCAGAAAGAACCCAAAAATAAGACCTTTCTA |
| | | AGATGCGAGGCCAAGAATTATTCTGGACGTTTCACCTGCTGGTGGCTG |
| | | ACGACAATCAGTACTGATTTGACATTCAGTGTCAAAAGCAGCAGAGG |
| | | CTCTTCTGACCCCCAAGGGGTGACGTGCGGAGCTGCTACACTCTCTGC |
| | | AGAGAGAGTCAGAGGGGACAACAAGGAGTATGAGTACTCAGTGGAG |
| | | TGCCAGGAGGACAGTGCCTGCCCAGCTGCTGAGGAGAGTCTGCCCAT |
| | | TGAGGTCATGGTGGATGCCGTTCACAAGCTCAAGTATGAAAACTACA |
| | | CCAGCAGCTTCTTCATCAGGGACATCATCAAACCTGACCCACCCAAG |
| | | AACTTGCAGCTGAAGCCATTAAAGAATTCTCGGCAGGTGGAGGTCAG |
| | | CTGGGAGTACCCTGACACCTGGAGTACTCCACATTCCTACTTCTCCCT |
| | | GACATTCTGCGTTCAGGTCCAGGGCAAGAGCAAGAGAGAAAAGAAA |
| | | GATAGAGTCTTCACGGACAAGACCTCAGCCACGGTCATCTGCCGCAA |
| | | AAATGCCAGCATTAGCGTGCGGGCCCAGGACCGCTACTATAGCTCAT |
| | | CTTGGAGCGAATGGGCATCTGTGCCCTGCAGTTGA |
| 16 | p40 (E81A, | ATGTGTCACCAGCAGTTGGTCATCTCTTGGTTTTCCCTGGTTTTTCTGG |
| | F82A)-15 aa | CATCTCCCCTCGTGGCCATATGGGAACTGAAGAAAGATGTTTATGTCG |
| | linker-p35 | TAGAATTGGATTGGTATCCGGATGCCCCTGGAGAAATGGTGGTCCTC |
| | | ACCTGTGACACCCCTGAAGAAGATGGTATCACCTGGACCTTGGACCA |
| | | GAGCAGTGAGGTCTTAGGCTCTGGCAAAACCCTGACCATCCAAGTCA |
| | | AAGCTGCCGGAGATGCTGGCCAGTACACCTGTCACAAAGGAGGCGAG |
| | | GTTCTAAGCCATTCGCTCCTGCTGCTTCACAAAAAGGAAGATGGAATT |
| | | TGGTCCACTGATATTTTAAAGGACCAGAAAGAACCCAAAAATAAGAC |
| | | CTTTCTAAGATGCGAGGCCAAGAATTATTCTGGACGTTTCACCTGCTG |
| | | GTGGCTGACGACAATCAGTACTGATTTGACATTCAGTGTCAAAAGCA |
| | | GCAGAGGCTCTTCTGACCCCCAAGGGGTGACGTGCGGAGCTGCTACA |
| | | CTCTCTGCAGAGAGAGTCAGAGGGGACAACAAGGAGTATGAGTACTC |
| | | AGTGGAGTGCCAGGAGGACAGTGCCTGCCCAGCTGCTGAGGAGAGTC |
| | | TGCCCATTGAGGTCATGGTGGATGCCGTTCACAAGCTCAAGTATGAA |
| | | AACTACACCAGCAGCTTCTTCATCAGGGACATCATCAAACCTGACCC |
| | | ACCCAAGAACTTGCAGCTGAAGCCATTAAAGAATTCTCGGCAGGTGG |
| | | AGGTCAGCTGGGAGTACCCTGACACCTGGAGTACTCCACATTCCTACT |
| | | TCTCCCTGACATTCTGCGTTCAGGTCCAGGGCAAGAGCAAGAGAGAA |
| | | AAGAAAGATAGAGTCTTCACGGACAAGACCTCAGCCACGGTCATCTG |
| | | CCGCAAAAATGCCAGCATTAGCGTGCGGGCCCAGGACCGCTACTATA |
| | | GCTCATCTTGGAGCGAATGGGCATCTGTGCCCTGCAGTGGTGGCGGT |
| | | GGATCTGGCGGTGGCGGTTCTGGCGGTGGAGGCTCTAGAAACCTCCC |
| | | CGTGGCCACTCCAGACCCAGGAATGTTCCCATGCCTTCACCACTCCCA |
| | | AAACCTGCTGAGGGCCGTCAGCAACATGCTCCAGAAGGCCAGACAAA |
| | | CTCTAGAATTTTACCCTTGCACTTCTGAAGAGATTGATCATGAAGATA |
| | | TCACAAAAGATAAAACCAGCACAGTGGAGGCCTGTTTACCATTGGAA |
| | | TTAACCAAGAATGAGAGTTGCCTAAATTCCAGAGAGACCTCTTTCATA |
| | | ACTAATGGGAGTTGCCTGGCCTCCAGAAAGACCTCTTTTATGATGGCC |
| | | CTGTGCCTTAGTAGTATTTATGAAGACTTGAAGATGTACCAGGTGGAG |
| | | TTCAAGACCATGAATGCAAAGCTTCTGATGGATCCTAAGAGGCAGAT |
| | | CTTTCTAGATCAAAACATGCTGGCAGTTATTGATGAGCTGATGCAGGC |
| | | CCTGAATTTCAACAGTGAGACTGTGCCACAAAAATCCTCCCTTGAAG |
| | | AACCGGATTTTTATAAAACTAAAATCAAGCTCTGCATACTTCTTCATG |
| | | CTTTCAGAATTCGGGCAGTGACTATTGATAGAGTGATGAGCTATCTGA |
| | | ATGCTTCCTGA |
| 17 | p40 (E81A, | ATGTGTCACCAGCAGTTGGTCATCTCTTGGTTTTCCCTGGTTTTTCTGG |
| | F82A, | CATCTCCCCTCGTGGCCATATGGGAACTGAAGAAAGATGTTTATGTCG |
| | K106A)-15 aa | TAGAATTGGATTGGTATCCGGATGCCCCTGGAGAAATGGTGGTCCTC |
| | linker-p35 | ACCTGTGACACCCCTGAAGAAGATGGTATCACCTGGACCTTGGACCA |
| | | GAGCAGTGAGGTCTTAGGCTCTGGCAAAACCCTGACCATCCAAGTCA |
| | | AAGCTGCCGGAGATGCTGGCCAGTACACCTGTCACAAAGGAGGCGAG |
| | | GTTCTAAGCCATTCGCTCCTGCTGCTTCACGCTAAGGAAGATGGAATT |
| | | TGGTCCACTGATATTTTAAAGGACCAGAAAGAACCCAAAAATAAGAC |
| | | CTTTCTAAGATGCGAGGCCAAGAATTATTCTGGACGTTTCACCTGCTG |
| | | GTGGCTGACGACAATCAGTACTGATTTGACATTCAGTGTCAAAAGCA |
| | | GCAGAGGCTCTTCTGACCCCCAAGGGGTGACGTGCGGAGCTGCTACA |
| | | CTCTCTGCAGAGAGAGTCAGAGGGGACAACAAGGAGTATGAGTACTC |
| | | AGTGGAGTGCCAGGAGGACAGTGCCTGCCCAGCTGCTGAGGAGAGTC |
| | | TGCCCATTGAGGTCATGGTGGATGCCGTTCACAAGCTCAAGTATGAA |
| | | AACTACACCAGCAGCTTCTTCATCAGGGACATCATCAAACCTGACCC |
| | | ACCCAAGAACTTGCAGCTGAAGCCATTAAAGAATTCTCGGCAGGTGG |
| | | AGGTCAGCTGGGAGTACCCTGACACCTGGAGTACTCCACATTCCTACT |
| | | TCTCCCTGACATTCTGCGTTCAGGTCCAGGGCAAGAGCAAGAGAGAA |
| | | AAGAAAGATAGAGTCTTCACGGACAAGACCTCAGCCACGGTCATCTG |
| | | CCGCAAAAATGCCAGCATTAGCGTGCGGGCCCAGGACCGCTACTATA |
| | | GCTCATCTTGGAGCGAATGGGCATCTGTGCCCTGCAGT GGT GGC GGT |
| | | GGATCTGGCGGTGGCGGTTCTGGCGGTGGAGGCTCTAGAAACCTCCC |
| | | CGTGGCCACTCCAGACCCAGGAATGTTCCCATGCCTTCACCACTCCCA |
| | | AAACCTGCTGAGGGCCGTCAGCAACATGCTCCAGAAGGCCAGACAAA |
| | | CTCTAGAATTTTACCCTTGCACTTCTGAAGAGATTGATCATGAAGATA |
| | | TCACAAAAGATAAAACCAGCACAGTGGAGGCCTGTTTACCATTGGAA |
| | | TTAACCAAGAATGAGAGTTGCCTAAATTCCAGAGAGACCTCTTTCATA |
| | | ACTAATGGGAGTTGCCTGGCCTCCAGAAAGACCTCTTTTATGATGGCC |
| | | CTGTGCCTTAGTAGTATTTATGAAGACTTGAAGATGTACCAGGTGGAG |
| | | TTCAAGACCATGAATGCAAAGCTTCTGATGGATCCTAAGAGGCAGAT |
| | | CTTTCTAGATCAAAACATGCTGGCAGTTATTGATGAGCTGATGCAGGC |
| | | CCTGAATTTCAACAGTGAGACTGTGCCACAAAAATCCTCCCTTGAAG |
| | | AACCGGATTTTTATAAAACTAAAATCAAGCTCTGCATACTTCTTCATG |
| | | CTTTCAGAATTCGGGCAGTGACTATTGATAGAGTGATGAGCTATCTGA |
| | | ATGCTTCCTGA |
| 18 | p40 (E81A, | ATGTGTCACCAGCAGTTGGTCATCTCTTGGTTTTCCCTGGTTTTTCTGG |
| | F82A, K106A, | CATCTCCCCTCGTGGCCATATGGGAACTGAAGAAAGATGTTTATGTCG |
| | K219A)-15 aa | TAGAATTGGATTGGTATCCGGATGCCCCTGGAGAAATGGTGGTCCTC |
| | linker-p35 | ACCTGTGACACCCCTGAAGAAGATGGTATCACCTGGACCTTGGACCA |
| | | GAGCAGTGAGGTCTTAGGCTCTGGCAAAACCCTGACCATCCAAGTCA |
| | | AAGCTGCCGGAGATGCTGGCCAGTACACCTGTCACAAAGGAGGCGAG |
| | | GTTCTAAGCCATTCGCTCCTGCTGCTTCACGCTAAGGAAGATGGAATT |
| | | TGGTCCACTGATATTTTAAAGGACCAGAAAGAACCCAAAAATAAGAC |
| | | CTTTCTAAGATGCGAGGCCAAGAATTATTCTGGACGTTTCACCTGCTG |
| | | GTGGCTGACGACAATCAGTACTGATTTGACATTCAGTGTCAAAAGCA |
| | | GCAGAGGCTCTTCTGACCCCCAAGGGGTGACGTGCGGAGCTGCTACA |
| | | CTCTCTGCAGAGAGAGTCAGAGGGGACAACAAGGAGTATGAGTACTC |
| | | AGTGGAGTGCCAGGAGGACAGTGCCTGCCCAGCTGCTGAGGAGAGTC |
| | | TGCCCATTGAGGTCATGGTGGATGCCGTTCACAAGCTCGCTTATGAAA |
| | | ACTACACCAGCAGCTTCTTCATCAGGGACATCATCAAACCTGACCCAC |
| | | CCAAGAACTTGCAGCTGAAGCCATTAAAGAATTCTCGGCAGGTGGAG |
| | | GTCAGCTGGGAGTACCCTGACACCTGGAGTACTCCACATTCCTACTTC |
| | | TCCCTGACATTCTGCGTTCAGGTCCAGGGCAAGAGCAAGAGAGAAAA |
| | | GAAAGATAGAGTCTTCACGGACAAGACCTCAGCCACGGTCATCTGCC |
| | | GCAAAAATGCCAGCATTAGCGTGCGGGCCCAGGACCGCTACTATAGC |
| | | TCATCTTGGAGCGAATGGGCATCTGTGCCCTGCAGT GGT GGC GGT |
| | | GGA TCT GGC GGT GGC GGT TCT GGC GGT GGA GGC |
| | | TCTAGAAACCTCCCCGTGGCCACTCCAGACCCAGGAATGTTCCCATGC |
| | | CTTCACCACTCCCAAAACCTGCTGAGGGCCGTCAGCAACATGCTCCA |
| | | GAAGGCCAGACAAACTCTAGAATTTTACCCTTGCACTTCTGAAGAGA |
| | | TTGATCATGAAGATATCACAAAAGATAAAACCAGCACAGTGGAGGCC |
| | | TGTTTACCATTGGAATTAACCAAGAATGAGAGTTGCCTAAATTCCAGA |
| | | GAGACCTCTTTCATAACTAATGGGAGTTGCCTGGCCTCCAGAAAGAC |
| | | CTCTTTTATGATGGCCCTGTGCCTTAGTAGTATTTATGAAGACTTGAA |
| | | GATGTACCAGGTGGAGTTCAAGACCATGAATGCAAAGCTTCTGATGG |
| | | ATCCTAAGAGGCAGATCTTTCTAGATCAAAACATGCTGGCAGTTATTG |
| | | ATGAGCTGATGCAGGCCCTGAATTTCAACAGTGAGACTGTGCCACAA |
| | | AAATCCTCCCTTGAAGAACCGGATTTTTATAAAACTAAAATCAAGCTC |
| | | TGCATACTTCTTCATGCTTTCAGAATTCGGGCAGTGACTATTGATAGA |
| | | GTGATGAGCTATCTGAATGCTTCCTGA |

TABLE 4
Amino Acid Sequences of scIL-12 Variant Domains

| SEQ ID | | |
|---|---|---|
| NO: | Description | Sequence |
| 19 | N terminus | MCHQQLVISWFSLVFLASPLVAIWELKKDVYVVELDWYPDAPGEMVVL |
| | p40 | TCDTPEEDGITWTLDQSSEVLGSGKTLTIQVKEFGDAGQYTCHKGGEVLS |
| | | HSLLLLHKKEDGIWSTDILKDQKEPKNKTFLRCEAKNYSGRFTCWWLTT |
| | | ISTDLTFSVKSSRGSSDPQGVTCGAATLSAERVRGDNKEYEYSVECQEDS |
| | | ACPAAEESLPIEVMVDAVHKLKYENYTSSFFIRDIIKPDPPKNLQLKPLKN |
| | | SRQVEVSWEYPDTWSTPHSYFSLTFCVQVQGKSKREKKDRVFTDKTSAT |
| | | VICRKNASISVRAQDRYYSSSWSEWASVPCS |
| 20 | C terminus | IWELKKDVYVVELDWYPDAPGEMVVLTCDTPEEDGITWTLDQSSEVLG |
| | p40 | SGKTLTIQVKEFGDAGQYTCHKGGEVLSHSLLLLHKKEDGIWSTDILKD |
| | | QKEPKNKTFLRCEAKNYSGRFTCWWLTTISTDLTFSVKSSRGSSDPQGVT |
| | | CGAATLSAERVRGDNKEYEYSVECQEDSACPAAEESLPIEVMVDAVHKL |
| | | KYENYTSSFFIRDIIKPDPPKNLQLKPLKNSRQVEVSWEYPDTWSTPHSYF |
| | | SLTFCVQVQGKSKREKKDRVFTDKTSATVICRKNASISVRAQDRYYSSS |
| | | WSEWASVPCS |
| 21 | N terminus | MCPARSLLLVATLVLLDHLSLARNLPVATPDPGMFPCLHHSQNLLRAVS |
| | p35 | NMLQKARQTLEFYPCTSEEIDHEDITKDKTSTVEACLPLELTKNESCLNSR |
| | | ETSFITNGSCLASRKTSFMMALCLSSIYEDLKMYQVEFKTMNAKLLMDP |
| | | KRQIFLDQNMLAVIDELMQALNFNSETVPQKSSLEEPDFYKTKIKLCILLH |
| | | AFRIRAVTIDRVMSYLNAS |
| 22 | C terminus | RNLPVATPDPGMFPCLHHSQNLLRAVSNMLQKARQTLEFYPCTSEEIDHE |
| | p35 | DITKDKTSTVEACLPLELTKNESCLNSRETSFITNGSCLASRKTSFMMALC |
| | | LSSIYEDLKMYQVEFKTMNAKLLMDPKRQIFLDQNMLAVIDELMQALNF |
| | | NSETVPQKSSLEEPDFYKTKIKLCILLHAFRIRAVTIDRVMSYLNAS |
| 23 | 15 amino acid | GGGGSGGGGSGGGGS |
| | linker | |
| 24 | 10 amino acid | GGGGSGGGGS |
| | linker | |
| 25 | 7 amino acid | GGGGGGS |
| | linker | |
| 26 | 5 amino acid | GGGGS |
| | linker | |

TABLE 5
DNA Sequences of scIL-12 Variant Domains

| SEQ ID | | |
|---|---|---|
| NO: | Description | Sequence |
| 27 | N terminus | ATGTGTCACCAGCAGTTGGTCATCTCTTGGTTTTCCCTGGTTTTTCTGG |
| | p40 | CATCTCCCCTCGTGGCCATATGGGAACTGAAGAAAGATGTTTATGTCG |
| | | TAGAATTGGATTGGTATCCGGATGCCCCTGGAGAAATGGTGGTCCTC |
| | | ACCTGTGACACCCCTGAAGAAGATGGTATCACCTGGACCTTGGACCA |
| | | GAGCAGTGAGGTCTTAGGCTCTGGCAAAACCCTGACCATCCAAGTCA |
| | | AAGAGTTTGGAGATGCTGGCCAGTACACCTGTCACAAAGGAGGCGAG |
| | | GTTCTAAGCCATTCGCTCCTGCTGCTTCACAAAAAGGAAGATGGAATT |
| | | TGGTCCACTGATATTTTAAAGGACCAGAAAGAACCCAAAAATAAGAC |
| | | CTTTCTAAGATGCGAGGCCAAGAATTATTCTGGACGTTTCACCTGCTG |
| | | GTGGCTGACGACAATCAGTACTGATTTGACATTCAGTGTCAAAAGCA |
| | | GCAGAGGCTCTTCTGACCCCCAAGGGGTGACGTGCGGAGCTGCTACA |
| | | CTCTCTGCAGAGAGAGTCAGAGGGGACAACAAGGAGTATGAGTACTC |
| | | AGTGGAGTGCCAGGAGGACAGTGCCTGCCCAGCTGCTGAGGAGAGTC |
| | | TGCCCATTGAGGTCATGGTGGATGCCGTTCACAAGCTCAAGTATGAA |
| | | AACTACACCAGCAGCTTCTTCATCAGGGACATCATCAAACCTGACCC |
| | | ACCCAAGAACTTGCAGCTGAAGCCATTAAAGAATTCTCGGCAGGTGG |
| | | AGGTCAGCTGGGAGTACCCTGACACCTGGAGTACTCCACATTCCTACT |
| | | TCTCCCTGACATTCTGCGTTCAGGTCCAGGGCAAGAGCAAGAGAGAA |
| | | AAGAAAGATAGAGTCTTCACGGACAAGACCTCAGCCACGGTCATCTG |
| | | CCGCAAAAATGCCAGCATTAGCGTGCGGGCCCAGGACCGCTACTATA |
| | | GCTCATCTTGGAGCGAATGGGCATCTGTGCCCTGCAGT |
| 28 | C terminus | ATATGGGAACTGAAGAAAGATGTTTATGTCGTAGAATTGGATTGGTA |
| | p40 | TCCGGATGCCCCTGGAGAAATGGTGGTCCTCACCTGTGACACCCCTGA |
| | | AGAAGATGGTATCACCTGGACCTTGGACCAGAGCAGTGAGGTCTTAG |
| | | GCTCTGGCAAAACCCTGACCATCCAAGTCAAAGAGTTTGGAGATGCT |
| | | GGCCAGTACACCTGTCACAAAGGAGGCGAGGTTCTAAGCCATTCGCT |
| | | CCTGCTGCTTCACAAAAAGGAAGATGGAATTTGGTCCACTGATATTTT |
| | | AAAGGACCAGAAAGAACCCAAAAATAAGACCTTTCTAAGATGCGAG |
| | | GCCAAGAATTATTCTGGACGTTTCACCTGCTGGTGGCTGACGACAATC |
| | | AGTACTGATTTGACATTCAGTGTCAAAAGCAGCAGAGGCTCTTCTGAC |
| | | CCCCAAGGGGTGACGTGCGGAGCTGCTACACTCTCTGCAGAGAGAGT |
| | | CAGAGGGGACAACAAGGAGTATGAGTACTCAGTGGAGTGCCAGGAG |
| | | GACAGTGCCTGCCCAGCTGCTGAGGAGAGTCTGCCCATTGAGGTCAT |
| | | GGTGGATGCCGTTCACAAGCTCAAGTATGAAAACTACACCAGCAGCT |
| | | TCTTCATCAGGGACATCATCAAACCTGACCCACCCAAGAACTTGCAG |
| | | CTGAAGCCATTAAAGAATTCTCGGCAGGTGGAGGTCAGCTGGGAGTA |
| | | CCCTGACACCTGGAGTACTCCACATTCCTACTTCTCCCTGACATTCTG |
| | | CGTTCAGGTCCAGGGCAAGAGCAAGAGAGAAAAGAAAGATAGAGTC |
| | | TTCACGGACAAGACCTCAGCCACGGTCATCTGCCGCAAAAATGCCAG |
| | | CATTAGCGTGCGGGCCCAGGACCGCTACTATAGCTCATCTTGGAGCG |
| | | AATGGGCATCTGTGCCCTGCAGTTGA |
| 29 | N terminus | ATGTGTCCAGCGCGCAGCCTCCTCCTTGTGGCTACCCTGGTCCTCCTG |
| | p35 | GACCACCTCAGTTTGGCCAGAAACCTCCCCGTGGCCACTCCAGACCC |
| | | AGGAATGTTCCCATGCCTTCACCACTCCCAAAACCTGCTGAGGGCCGT |
| | | CAGCAACATGCTCCAGAAGGCCAGACAAACTCTAGAATTTTACCCTT |
| | | GCACTTCTGAAGAGATTGATCATGAAGATATCACAAAAGATAAAACC |
| | | AGCACAGTGGAGGCCTGTTTACCATTGGAATTAACCAAGAATGAGAG |
| | | TTGCCTAAATTCCAGAGAGACCTCTTTCATAACTAATGGGAGTTGCCT |
| | | GGCCTCCAGAAAGACCTCTTTTATGATGGCCCTGTGCCTTAGTAGTAT |
| | | TTATGAAGACTTGAAGATGTACCAGGTGGAGTTCAAGACCATGAATG |
| | | CAAAGCTTCTGATGGATCCTAAGAGGCAGATCTTTCTAGATCAAAAC |
| | | ATGCTGGCAGTTATTGATGAGCTGATGCAGGCCCTGAATTTCAACAGT |
| | | GAGACTGTGCCACAAAAATCCTCCCTTGAAGAACCGGATTTTTATAA |
| | | AACTAAAATCAAGCTCTGCATACTTCTTCATGCTTTCAGAATTCGGGC |
| | | AGTGACTATTGATAGAGTGATGAGCTATCTGAATGCTTCC |
| 30 | C terminus | AGAAACCTCCCCGTGGCCACTCCAGACCCAGGAATGTTCCCATGCCTT |
| | p35 | CACCACTCCCAAAACCTGCTGAGGGCCGTCAGCAACATGCTCCAGAA |
| | | GGCCAGACAAACTCTAGAATTTTACCCTTGCACTTCTGAAGAGATTGA |
| | | TCATGAAGATATCACAAAAGATAAAACCAGCACAGTGGAGGCCTGTT |
| | | TACCATTGGAATTAACCAAGAATGAGAGTTGCCTAAATTCCAGAGAG |
| | | ACCTCTTTCATAACTAATGGGAGTTGCCTGGCCTCCAGAAAGACCTCT |
| | | TTTATGATGGCCCTGTGCCTTAGTAGTATTTATGAAGACTTGAAGATG |
| | | TACCAGGTGGAGTTCAAGACCATGAATGCAAAGCTTCTGATGGATCC |
| | | TAAGAGGCAGATCTTTCTAGATCAAAACATGCTGGCAGTTATTGATG |
| | | AGCTGATGCAGGCCCTGAATTTCAACAGTGAGACTGTGCCACAAAAA |
| | | TCCTCCCTTGAAGAACCGGATTTTTATAAAACTAAAATCAAGCTCTGC |
| | | ATACTTCTTCATGCTTTCAGAATTCGGGCAGTGACTATTGATAGAGTG |
| | | ATGAGCTATCTGAATGCTTCCTGA |
| 31 | 15 amino acid | GGT GGC GGT GGA TCT GGC GGT GGC GGT TCT GGC GGT GGA GGC |
| | linker | TCT |
| 32 | 10 amino acid | GGT GGC GGT GGA TCT GGC GGT GGC GGT TCT |
| | linker | |
| 33 | 7 amino acid | GGC GGA GGT GGA GGC GGT TCT |
| | linker | |
| 34 | 5 amino acid | GGC GGA GGT GGA TCT |
| | linker | |

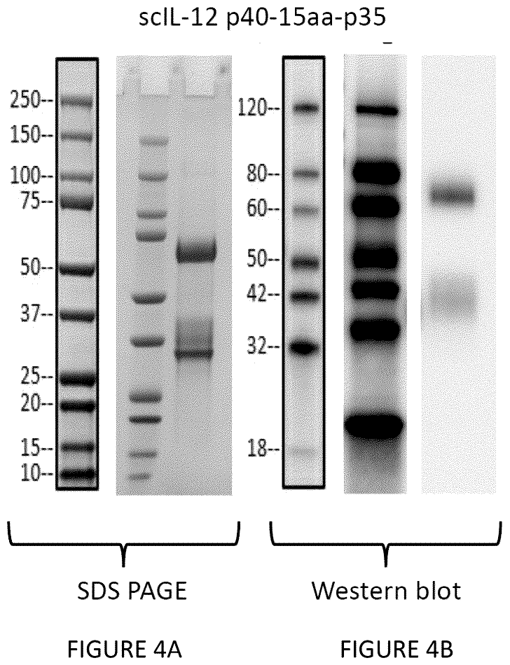

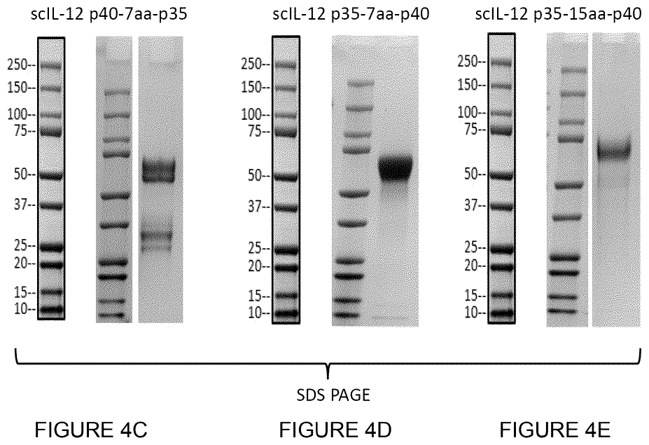

Recombinant IL-12 single chain variants were produced in CHO cells. The gene of interest was cloned into the pcDNA expression vector containing a strong promoter and a C-terminal His tag. The vector was transfected into CHO cells, and supernatant harvested after 5 days. Protein was purified using a His-tag affinity chromatography column. The purified protein was quantified, and its purity analyzed by SDS PAGE and Western blotting (FIG. 4A-4E). Detection of the antibodies was carried out using a Rabbit anti-His antibody (genscript). Western blot confirmed the identity of the proteins, and SDS PAGE confirmed that the proteins were at their expected molecular weight (approximately 70 kDa).

The constructs listed in Table 2-3 are humanized construct. Murine constructs were also generated; CM133 is the murine version of CM068, CM131 is the murine version of CM065, and CM134 is the murine version of CM064. Their sequences are as shown in Tables 6-9.

TABLE 6
Amino Acid Sequences of Murine scIL-12 Variants

| SEQ ID | | |
|---|---|---|
| NO: | Description | Sequence |
| 35 | CM133 | MCPQKLTISWFAIVLLVSPLMAMWELEKDVYVVEVDWTPDAPGETVNL |
| | | TCDTPEEDDITWTSDQRHGVIGSGKTLTITVKAALDAGQYTCHKGGETLS |
| | | HSHLLLHaKENGIWSTEILKNFKNKTFLKCEAPNYSGRFTCSWLVQRNM |
| | | DLKFNIKSSSSSPDSRAVTCGMASLSAEKVTLDQRDYEKYSVSCQEDVTC |
| | | PTAEETLPIELALEARQQNKYENYSTSFFIRDIIKPDPPKNLQMKPLKNSQV |
| | | EVSWEYPDSWSTPHSYFSLKFFVRIQRKKEKMKETEEGCNQKGAFLVEK |
| | | TSTEVQCKGGNVCVQAQDRYYNSSCSKWACVPCRVRSGGGGSGGGGS |
| | | GGGGSRVIPVSGPARCLSQSRNLLKTTDDMVKTAREKLKHYSCTAEDID |
| | | HEDITRDQTSTLKTCLPLELHKNESCLATRETSSTTRGSCLPPQKTSLMMT |
| | | LCLGSIYEDLKMYQTEFQAINAALQNHNHQQIILDKGMLVAIDELMQSL |
| | | NHNGETLRQKPPVGEADPYRVKMKLCILLHAFSTRVVTINRVMGYLSSA |
| 36 | CM131 | MCPQKLTISWFAIVLLVSPLMAMWELEKDVYVVEVDWTPDAPGETVNL |
| | | TCDTPEEDDITWTSDQRHGVIGSGKTLTITVKAALDAGQYTCHKGGETLS |
| | | HSHLLLHKKENGIWSTEILKNFKNKTFLKCEAPNYSGRFTCSWLVQRNM |
| | | DLKFNIKSSSSSPDSRAVTCGMASLSAEKVTLDQRDYEKYSVSCQEDVTC |
| | | PTAEETLPIELALEARQQNAYENYSTSFFIRDIIKPDPPKNLQMKPLKNSQ |
| | | VEVSWEYPDSWSTPHSYFSLKFFVRIQRKKEKMKETEEGCNQKGAFLVE |
| | | KTSTEVQCKGGNVCVQAQDRYYNSSCSKWACVPCRVRSGGGGSGGGG |
| | | SGGGGSRVIPVSGPARCLSQSRNLLKTTDDMVKTAREKLKHYSCTAEDI |
| | | DHEDITRDQTSTLKTCLPLELHKNESCLATRETSSTTRGSCLPPQKTSLM |
| | | MTLCLGSIYEDLKMYQTEFQAINAALQNHNHQQIILDKGMLVAIDELMQ |
| | | SLNHNGETLRQKPPVGEADPYRVKMKLCILLHAFSTRVVTINRVMGYLS |
| | | SA |
| 37 | CM134 | MCQSRYLLFLATLALLNHLSLARVIPVSGPARCLSQSRNLLKTTDDMVK |
| | | TAREKLKHYSCTAEDIDHEDITRDQTSTLKTCLPLELHKNESCLATRETSS |
| | | TTRGSCLPPQKTSLMMTLCLGSIYEDLKMYQTEFQAINAALQNHNHQQII |
| | | LDKGMLVAIDELMQSLNHNGETLRQKPPVGEADPYRVKMKLCILLHAFS |
| | | TRVVTINRVMGYLSSAGGGGGGSMWELEKDVYVVEVDWTPDAPGETV |
| | | NLTCDTPEEDDITWTSDQRHGVIGSGKTLTITVKEFLDAGQYTCHKGGET |
| | | LSHSHLLLHKKENGIWSTEILKNFKNKTFLKCEAPNYSGRFTCSWLVQRN |
| | | MDLKFNIKSSSSSPDSRAVTCGMASLSAEKVTLDQRDYEKYSVSCQEDV |
| | | TCPTAEETLPIELALEARQQNKYENYSTSFFIRDIIKPDPPKNLQMKPLKNS |
| | | QVEVSWEYPDSWSTPHSYFSLKFFVRIQRKKEKMKETEEGCNQKGAFLV |
| | | EKTSTEVQCKGGNVCVQAQDRYYNSSCSKWACVPCRVRS |

TABLE 7
DNA Sequences of Murine scIL-12 Variants

| SEQ ID | | |
|---|---|---|
| NO: | Description | Sequence |
| 38 | CM133 | ATGTGTCCTCAGAAGCTAACCATCTCCTGGTTTGCCATCGTTTTGCTG |
| | | GTGTCTCCACTCATGGCCATGTGGGAGCTGGAGAAAGACGTTTATGTT |
| | | GTAGAGGTGGACTGGACTCCCGATGCCCCTGGAGAAACAGTGAACCT |
| | | CACCTGTGACACGCCTGAAGAAGATGACATCACCTGGACCTCAGACC |
| | | AGAGACATGGAGTCATAGGCTCTGGAAAGACCCTGACCATCACTGTC |
| | | AAAgctgccCTAGATGCTGGCCAGTACACCTGCCACAAAGGAGGCGAGA |
| | | CTCTGAGCCACTCACATCTGCTGCTCCACAAGAAGGAAAATGGAATT |
| | | TGGTCCACTGAAATTTTAAAAAATTTCAAAAACAAGACTTTCCTGAAG |
| | | TGTGAAGCACCAAATTACTCCGGACGGTTCACGTGCTCATGGCTGGTG |
| | | CAAAGAAACATGGACTTGAAGTTCAACATCAAGAGCAGTAGCAGTTC |
| | | CCCTGACTCTCGGGCAGTGACATGTGGAATGGCGTCTCTGTCTGCAGA |
| | | GAAGGTCACACTGGACCAAAGGGACTATGAGAAGTATTCAGTGTCCT |
| | | GCCAGGAGGATGTCACCTGCCCAACTGCCGAGGAGACCCTGCCCATT |
| | | GAACTGGCGTTGGAAGCACGGCAGCAGAATgctTATGAGAACTACAGC |
| | | ACCAGCTTCTTCATCAGGGACATCATCAAACCAGACCCGCCCAAGAA |
| | | CTTGCAGATGAAGCCTTTGAAGAACTCACAGGTGGAGGTCAGCTGGG |
| | | AGTACCCTGACTCCTGGAGCACTCCCCATTCCTACTTCTCCCTCAAGT |
| | | TCTTTGTTCGAATCCAGCGCAAGAAAGAAAAGATGAAGGAGACAGAG |
| | | GAGGGGTGTAACCAGAAAGGTGCGTTCCTCGTAGAGAAGACATCTAC |
| | | CGAAGTCCAATGCAAAGGCGGGAATGTCTGCGTGCAAGCTCAGGATC |
| | | GCTATTACAATTCCTCATGCAGCAAGTGGGCATGTGTTCCCTGCAGGG |
| | | TCCGATCC GGT GGC GGT GGA TCT GGC GGT GGC GGT TCT GGC GGT |
| | | GGA GGC |
| | | TCTAGGGTCATTCCAGTCTCTGGACCTGCCAGGTGTCTTAGCCAGTCC |
| | | CGAAACCTGCTGAAGACCACAGATGACATGGTGAAGACGGCCAGAG |
| | | AAAAACTGAAACATTATTCCTGCACTGCTGAAGACATCGATCATGAA |
| | | GACATCACACGGGACCAAACCAGCACATTGAAGACCTGTTTACCACT |
| | | GGAACTACACAAGAACGAGAGTTGCCTGGCTACTAGAGAGACTTCTT |
| | | CCACAACAAGAGGGAGCTGCCTGCCCCCACAGAAGACGTCTTTGATG |
| | | ATGACCCTGTGCCTTGGTAGCATCTATGAGGACTTGAAGATGTACCAG |
| | | ACAGAGTTCCAGGCCATCAACGCAGCACTTCAGAATCACAACCATCA |
| | | GCAGATCATTCTAGACAAGGGCATGCTGGTGGCCATCGATGAGCTGA |
| | | TGCAGTCTCTGAATCATAATGGCGAGACTCTGCGCCAGAAACCTCCTG |
| | | TGGGAGAAGCAGACCCTTACAGAGTGAAAATGAAGCTCTGCATCCTG |
| | | CTTCACGCCTTCAGCACCCGCGTCGTGACCATCAACAGGGTGATGGG |
| | | CTATCTGAGCTCCGCCTAA |
| 39 | CM131 | ATGTGTCCTCAGAAGCTAACCATCTCCTGGTTTGCCATCGTTTTGCTG |
| | | GTGTCTCCACTCATGGCCATGTGGGAGCTGGAGAAAGACGTTTATGTT |
| | | GTAGAGGTGGACTGGACTCCCGATGCCCCTGGAGAAACAGTGAACCT |
| | | CACCTGTGACACGCCTGAAGAAGATGACATCACCTGGACCTCAGACC |
| | | AGAGACATGGAGTCATAGGCTCTGGAAAGACCCTGACCATCACTGTC |
| | | AAAGAGTTTCTAGATGCTGGCCAGTACACCTGCCACAAAGGAGGCGA |
| | | GACTCTGAGCCACTCACATCTGCTGCTCCACAAGAAGGAAAATGGAA |
| | | TTTGGTCCACTGAAATTTTAAAAAATTTCAAAAACAAGACTTTCCTGA |
| | | AGTGTGAAGCACCAAATTACTCCGGACGGTTCACGTGCTCATGGCTG |
| | | GTGCAAAGAAACATGGACTTGAAGTTCAACATCAAGAGCAGTAGCAG |
| | | TTCCCCTGACTCTCGGGCAGTGACATGTGGAATGGCGTCTCTGTCTGC |
| | | AGAGAAGGTCACACTGGACCAAAGGGACTATGAGAAGTATTCAGTGT |
| | | CCTGCCAGGAGGATGTCACCTGCCCAACTGCCGAGGAGACCCTGCCC |
| | | ATTGAACTGGCGTTGGAAGCACGGCAGCAGAATAAATATGAGAACTA |
| | | CAGCACCAGCTTCTTCATCAGGGACATCATCAAACCAGACCCGCCCA |
| | | AGAACTTGCAGATGAAGCCTTTGAAGAACTCACAGGTGGAGGTCAGC |
| | | TGGGAGTACCCTGACTCCTGGAGCACTCCCCATTCCTACTTCTCCCTC |
| | | AAGTTCTTTGTTCGAATCCAGCGCAAGAAAGAAAAGATGAAGGAGAC |
| | | AGAGGAGGGGTGTAACCAGAAAGGTGCGTTCCTCGTAGAGAAGACAT |
| | | CTACCGAAGTCCAATGCAAAGGCGGGAATGTCTGCGTGCAAGCTCAG |
| | | GATCGCTATTACAATTCCTCATGCAGCAAGTGGGCATGTGTTCCCTGC |
| | | AGGGTCCGATCC GGT GGC GGT GGA TCT GGC GGT GGC GGT TCT |
| | | GGC GGT GGA GGC |
| | | TCTAGGGTCATTCCAGTCTCTGGACCTGCCAGGTGTCTTAGCCAGTCC |
| | | CGAAACCTGCTGAAGACCACAGATGACATGGTGAAGACGGCCAGAG |
| | | AAAAACTGAAACATTATTCCTGCACTGCTGAAGACATCGATCATGAA |
| | | GACATCACACGGGACCAAACCAGCACATTGAAGACCTGTTTACCACT |
| | | GGAACTACACAAGAACGAGAGTTGCCTGGCTACTAGAGAGACTTCTT |
| | | CCACAACAAGAGGGAGCTGCCTGCCCCCACAGAAGACGTCTTTGATG |
| | | ATGACCCTGTGCCTTGGTAGCATCTATGAGGACTTGAAGATGTACCAG |
| | | ACAGAGTTCCAGGCCATCAACGCAGCACTTCAGAATCACAACCATCA |
| | | GCAGATCATTCTAGACAAGGGCATGCTGGTGGCCATCGATGAGCTGA |
| | | TGCAGTCTCTGAATCATAATGGCGAGACTCTGCGCCAGAAACCTCCTG |
| | | TGGGAGAAGCAGACCCTTACAGAGTGAAAATGAAGCTCTGCATCCTG |
| | | CTTCACGCCTTCAGCACCCGCGTCGTGACCATCAACAGGGTGATGGG |
| | | CTATCTGAGCTCCGCCTAA |
| 40 | CM134 | ATGTGTCAATCACGCTACCTCCTCTTTTTGGCCACCCTTGCCCTCCTAA |
| | | ACCACCTCAGTTTGGCCAGGGTCATTCCAGTCTCTGGACCTGCCAGGT |
| | | GTCTTAGCCAGTCCCGAAACCTGCTGAAGACCACAGATGACATGGTG |
| | | AAGACGGCCAGAGAAAAACTGAAACATTATTCCTGCACTGCTGAAGA |
| | | CATCGATCATGAAGACATCACACGGGACCAAACCAGCACATTGAAGA |
| | | CCTGTTTACCACTGGAACTACACAAGAACGAGAGTTGCCTGGCTACT |
| | | AGAGAGACTTCTTCCACAACAAGAGGGAGCTGCCTGCCCCCACAGAA |
| | | GACGTCTTTGATGATGACCCTGTGCCTTGGTAGCATCTATGAGGACTT |
| | | GAAGATGTACCAGACAGAGTTCCAGGCCATCAACGCAGCACTTCAGA |
| | | ATCACAACCATCAGCAGATCATTCTAGACAAGGGCATGCTGGTGGCC |
| | | ATCGATGAGCTGATGCAGTCTCTGAATCATAATGGCGAGACTCTGCG |
| | | CCAGAAACCTCCTGTGGGAGAAGCAGACCCTTACAGAGTGAAAATGA |
| | | AGCTCTGCATCCTGCTTCACGCCTTCAGCACCCGCGTCGTGACCATCA |
| | | ACAGGGTGATGGGCTATCTGAGCTCCGCC GGC GGA GGT GGA GGC |
| | | GGT |
| | | TCTATGTGGGAGCTGGAGAAAGACGTTTATGTTGTAGAGGTGGACTG |
| | | GACTCCCGATGCCCCTGGAGAAACAGTGAACCTCACCTGTGACACGC |
| | | CTGAAGAAGATGACATCACCTGGACCTCAGACCAGAGACATGGAGTC |
| | | ATAGGCTCTGGAAAGACCCTGACCATCACTGTCAAAGAGTTTCTAGA |
| | | TGCTGGCCAGTACACCTGCCACAAAGGAGGCGAGACTCTGAGCCACT |
| | | CACATCTGCTGCTCCACAAGAAGGAAAATGGAATTTGGTCCACTGAA |
| | | ATTTTAAAAAATTTCAAAAACAAGACTTTCCTGAAGTGTGAAGCACC |
| | | AAATTACTCCGGACGGTTCACGTGCTCATGGCTGGTGCAAAGAAACA |
| | | TGGACTTGAAGTTCAACATCAAGAGCAGTAGCAGTTCCCCTGACTCTC |
| | | GGGCAGTGACATGTGGAATGGCGTCTCTGTCTGCAGAGAAGGTCACA |
| | | CTGGACCAAAGGGACTATGAGAAGTATTCAGTGTCCTGCCAGGAGGA |
| | | TGTCACCTGCCCAACTGCCGAGGAGACCCTGCCCATTGAACTGGCGTT |
| | | GGAAGCACGGCAGCAGAATAAATATGAGAACTACAGCACCAGCTTCT |
| | | TCATCAGGGACATCATCAAACCAGACCCGCCCAAGAACTTGCAGATG |
| | | AAGCCTTTGAAGAACTCACAGGTGGAGGTCAGCTGGGAGTACCCTGA |
| | | CTCCTGGAGCACTCCCCATTCCTACTTCTCCCTCAAGTTCTTTGTTCGA |
| | | ATCCAGCGCAAGAAAGAAAAGATGAAGGAGACAGAGGAGGGGTGTA |
| | | ACCAGAAAGGTGCGTTCCTCGTAGAGAAGACATCTACCGAAGTCCAA |
| | | TGCAAAGGCGGGAATGTCTGCGTGCAAGCTCAGGATCGCTATTACAA |
| | | TTCCTCATGCAGCAAGTGGGCATGTGTTCCCTGCAGGGTCCGATCCTA |
| | | A |

TABLE 8
Amino Acid Sequences of Murine scIL-12 Variant Domains

| SEQ ID | | |
|---|---|---|
| NO: | Description | Sequence |
| 41 | N Terminus | MCPQKLTISWFAIVLLVSPLMAMWELEKDVYVVEVDWTPDAPGETVNL |
| | Murine p40 | TCDTPEEDDITWTSDQRHGVIGSGKTLTITVKAALDAGQYTCHKGGETLS |
| | (E81A, F82A, | HSHLLLHaKENGIWSTEILKNFKNKTFLKCEAPNYSGRFTCSWLVQRNM |
| | K106A) | DLKFNIKSSSSSPDSRAVTCGMASLSAEKVTLDQRDYEKYSVSCQEDVTC |
| | | PTAEETLPIELALEARQQNKYENYSTSFFIRDIIKPDPPKNLQMKPLKNSQV |
| | | EVSWEYPDSWSTPHSYFSLKFFVRIQRKKEKMKETEEGCNQKGAFLVEK |
| | | TSTEVQCKGGNVCVQAQDRYYNSSCSKWACVPCRVRS |
| 42 | C Terminus | RVIPVSGPARCLSQSRNLLKTTDDMVKTAREKLKHYSCTAEDIDHEDITR |
| | Murine p35 | DQTSTLKTCLPLELHKNESCLATRETSSTTRGSCLPPQKTSLMMTLCLGSI |
| | | YEDLKMYQTEFQAINAALQNHNHQQIILDKGMLVAIDELMQSLNHNGET |
| | | LRQKPPVGEADPYRVKMKLCILLHAFSTRVVTINRVMGYLSSA |
| 43 | N Terminus | MCPQKLTISWFAIVLLVSPLMAMWELEKDVYVVEVDWTPDAPGETVNL |
| | Murine p40 | TCDTPEEDDITWTSDQRHGVIGSGKTLTITVKEFLDAGQYTCHKGGETLS |
| | | HSHLLLHKKENGIWSTEILKNFKNKTFLKCEAPNYSGRFTCSWLVQRNM |
| | | DLKFNIKSSSSSPDSRAVTCGMASLSAEKVTLDQRDYEKYSVSCQEDVTC |
| | | PTAEETLPIELALEARQQNKYENYSTSFFIRDIIKPDPPKNLQMKPLKNSQ |
| | | VEVSWEYPDSWSTPHSYFSLKFFVRIQRKKEKMKETEEGCNQKGAFLVE |
| | | KTSTEVQCKGGNVCVQAQDRYYNSSCSKWACVPCRVRS |
| 44 | N Terminus | MCQSRYLLFLATLALLNHLSLARVIPVSGPARCLSQSRNLLKTTDDMVK |
| | Murine p35 | TAREKLKHYSCTAEDIDHEDITRDQTSTLKTCLPLELHKNESCLATRETSS |
| | | TTRGSCLPPQKTSLMMTLCLGSIYEDLKMYQTEFQAINAALQNHNHQQII |
| | | LDKGMLVAIDELMQSLNHNGETLRQKPPVGEADPYRVKMKLCILLHAFS |
| | | TRVVTINRVMGYLSSA |
| 45 | C Terminus | MWELEKDVYVVEVDWTPDAPGETVNLTCDTPEEDDITWTSDQRHGVIG |
| | Murine p40 | SGKTLTITVKEFLDAGQYTCHKGGETLSHSHLLLHKKENGIWSTEILKNF |
| | | KNKTFLKCEAPNYSGRFTCSWLVQRNMDLKFNIKSSSSSPDSRAVTCGM |
| | | ASLSAEKVTLDQRDYEKYSVSCQEDVTCPTAEETLPIELALEARQQNKYE |
| | | NYSTSFFIRDIIKPDPPKNLQMKPLKNSQVEVSWEYPDSWSTPHSYFSLKF |
| | | FVRIQRKKEKMKETEEGCNQKGAFLVEKTSTEVQCKGGNVCVQAQDRY |
| | | YNSSCSKWACVPCRVRS |

TABLE 9
DNA Sequences of Murine scIL-12 Variant Domains

| SEQ ID | | |
|---|---|---|
| NO: | Description | Sequence |
| 46 | N Terminus | ATGTGTCCTCAGAAGCTAACCATCTCCTGGTTTGCCATCGTTTTGCTG |
| | Murine p40 | GTGTCTCCACTCATGGCCATGTGGGAGCTGGAGAAAGACGTTTATGTT |
| | (E81A, F82A, | GTAGAGGTGGACTGGACTCCCGATGCCCCTGGAGAAACAGTGAACCT |
| | K106A) | CACCTGTGACACGCCTGAAGAAGATGACATCACCTGGACCTCAGACC |
| | | AGAGACATGGAGTCATAGGCTCTGGAAAGACCCTGACCATCACTGTC |
| | | AAAgctgccCTAGATGCTGGCCAGTACACCTGCCACAAAGGAGGCGAGA |
| | | CTCTGAGCCACTCACATCTGCTGCTCCACGCCAAGGAAAATGGAATTT |
| | | GGTCCACTGAAATTTTAAAAAATTTCAAAAACAAGACTTTCCTGAAGT |
| | | GTGAAGCACCAAATTACTCCGGACGGTTCACGTGCTCATGGCTGGTG |
| | | CAAAGAAACATGGACTTGAAGTTCAACATCAAGAGCAGTAGCAGTTC |
| | | CCCTGACTCTCGGGCAGTGACATGTGGAATGGCGTCTCTGTCTGCAGA |
| | | GAAGGTCACACTGGACCAAAGGGACTATGAGAAGTATTCAGTGTCCT |
| | | GCCAGGAGGATGTCACCTGCCCAACTGCCGAGGAGACCCTGCCCATT |
| | | GAACTGGCGTTGGAAGCACGGCAGCAGAATAAATATGAGAACTACA |
| | | GCACCAGCTTCTTCATCAGGGACATCATCAAACCAGACCCGCCCAAG |
| | | AACTTGCAGATGAAGCCTTTGAAGAACTCACAGGTGGAGGTCAGCTG |
| | | GGAGTACCCTGACTCCTGGAGCACTCCCCATTCCTACTTCTCCCTCAA |
| | | GTTCTTTGTTCGAATCCAGCGCAAGAAAGAAAAGATGAAGGAGACAG |
| | | AGGAGGGGTGTAACCAGAAAGGTGCGTTCCTCGTAGAGAAGACATCT |
| | | ACCGAAGTCCAATGCAAAGGCGGGAATGTCTGCGTGCAAGCTCAGGA |
| | | TCGCTATTACAATTCCTCATGCAGCAAGTGGGCATGTGTTCCCTGCAG |
| | | GGTCCGATCC |
| 47 | C Terminus | AGGGTCATTCCAGTCTCTGGACCTGCCAGGTGTCTTAGCCAGTCCCGA |
| | Murine p35 | AACCTGCTGAAGACCACAGATGACATGGTGAAGACGGCCAGAGAAA |
| | | AACTGAAACATTATTCCTGCACTGCTGAAGACATCGATCATGAAGAC |
| | | ATCACACGGGACCAAACCAGCACATTGAAGACCTGTTTACCACTGGA |
| | | ACTACACAAGAACGAGAGTTGCCTGGCTACTAGAGAGACTTCTTCCA |
| | | CAACAAGAGGGAGCTGCCTGCCCCCACAGAAGACGTCTTTGATGATG |
| | | ACCCTGTGCCTTGGTAGCATCTATGAGGACTTGAAGATGTACCAGAC |
| | | AGAGTTCCAGGCCATCAACGCAGCACTTCAGAATCACAACCATCAGC |
| | | AGATCATTCTAGACAAGGGCATGCTGGTGGCCATCGATGAGCTGATG |
| | | CAGTCTCTGAATCATAATGGCGAGACTCTGCGCCAGAAACCTCCTGTG |
| | | GGAGAAGCAGACCCTTACAGAGTGAAAATGAAGCTCTGCATCCTGCT |
| | | TCACGCCTTCAGCACCCGCGTCGTGACCATCAACAGGGTGATGGGCT |
| | | ATCTGAGCTCCGCCTAA |
| 48 | N Terminus | ATGTGTCCTCAGAAGCTAACCATCTCCTGGTTTGCCATCGTTTTGCTG |
| | Murine p40 | GTGTCTCCACTCATGGCCATGTGGGAGCTGGAGAAAGACGTTTATGTT |
| | | GTAGAGGTGGACTGGACTCCCGATGCCCCTGGAGAAACAGTGAACCT |
| | | CACCTGTGACACGCCTGAAGAAGATGACATCACCTGGACCTCAGACC |
| | | AGAGACATGGAGTCATAGGCTCTGGAAAGACCCTGACCATCACTGTC |
| | | AAAGAGTTTCTAGATGCTGGCCAGTACACCTGCCACAAAGGAGGCGA |
| | | GACTCTGAGCCACTCACATCTGCTGCTCCACAAGAAGGAAAATGGAA |
| | | TTTGGTCCACTGAAATTTTAAAAAATTTCAAAAACAAGACTTTCCTGA |
| | | AGTGTGAAGCACCAAATTACTCCGGACGGTTCACGTGCTCATGGCTG |
| | | GTGCAAAGAAACATGGACTTGAAGTTCAACATCAAGAGCAGTAGCAG |
| | | TTCCCCTGACTCTCGGGCAGTGACATGTGGAATGGCGTCTCTGTCTGC |
| | | AGAGAAGGTCACACTGGACCAAAGGGACTATGAGAAGTATTCAGTGT |
| | | CCTGCCAGGAGGATGTCACCTGCCCAACTGCCGAGGAGACCCTGCCC |
| | | ATTGAACTGGCGTTGGAAGCACGGCAGCAGAATAAATATGAGAACTA |
| | | CAGCACCAGCTTCTTCATCAGGGACATCATCAAACCAGACCCGCCCA |
| | | AGAACTTGCAGATGAAGCCTTTGAAGAACTCACAGGTGGAGGTCAGC |
| | | TGGGAGTACCCTGACTCCTGGAGCACTCCCCATTCCTACTTCTCCCTC |
| | | AAGTTCTTTGTTCGAATCCAGCGCAAGAAAGAAAAGATGAAGGAGAC |
| | | AGAGGAGGGGTGTAACCAGAAAGGTGCGTTCCTCGTAGAGAAGACAT |
| | | CTACCGAAGTCCAATGCAAAGGCGGGAATGTCTGCGTGCAAGCTCAG |
| | | GATCGCTATTACAATTCCTCATGCAGCAAGTGGGCATGTGTTCCCTGC |
| | | AGGGTCCGATCC |
| 49 | N Terminus | ATGTGTCAATCACGCTACCTCCTCTTTTTGGCCACCCTTGCCCTCCTAA |
| | Murine p35 | ACCACCTCAGTTTGGCCAGGGTCATTCCAGTCTCTGGACCTGCCAGGT |
| | | GTCTTAGCCAGTCCCGAAACCTGCTGAAGACCACAGATGACATGGTG |
| | | AAGACGGCCAGAGAAAAACTGAAACATTATTCCTGCACTGCTGAAGA |
| | | CATCGATCATGAAGACATCACACGGGACCAAACCAGCACATTGAAGA |
| | | CCTGTTTACCACTGGAACTACACAAGAACGAGAGTTGCCTGGCTACT |
| | | AGAGAGACTTCTTCCACAACAAGAGGGAGCTGCCTGCCCCCACAGAA |
| | | GACGTCTTTGATGATGACCCTGTGCCTTGGTAGCATCTATGAGGACTT |
| | | GAAGATGTACCAGACAGAGTTCCAGGCCATCAACGCAGCACTTCAGA |
| | | ATCACAACCATCAGCAGATCATTCTAGACAAGGGCATGCTGGTGGCC |
| | | ATCGATGAGCTGATGCAGTCTCTGAATCATAATGGCGAGACTCTGCG |
| | | CCAGAAACCTCCTGTGGGAGAAGCAGACCCTTACAGAGTGAAAATGA |
| | | AGCTCTGCATCCTGCTTCACGCCTTCAGCACCCGCGTCGTGACCATCA |
| | | ACAGGGTGATGGGCTATCTGAGCTCCGCC |
| 50 | C Terminus | ATGTGGGAGCTGGAGAAAGACGTTTATGTTGTAGAGGTGGACTGGAC |
| | Murine p40 | TCCCGATGCCCCTGGAGAAACAGTGAACCTCACCTGTGACACGCCTG |
| | | AAGAAGATGACATCACCTGGACCTCAGACCAGAGACATGGAGTCATA |
| | | GGCTCTGGAAAGACCCTGACCATCACTGTCAAAGAGTTTCTAGATGCT |
| | | GGCCAGTACACCTGCCACAAAGGAGGCGAGACTCTGAGCCACTCACA |
| | | TCTGCTGCTCCACAAGAAGGAAAATGGAATTTGGTCCACTGAAATTTT |
| | | AAAAAATTTCAAAAACAAGACTTTCCTGAAGTGTGAAGCACCAAATT |
| | | ACTCCGGACGGTTCACGTGCTCATGGCTGGTGCAAAGAAACATGGAC |
| | | TTGAAGTTCAACATCAAGAGCAGTAGCAGTTCCCCTGACTCTCGGGC |
| | | AGTGACATGTGGAATGGCGTCTCTGTCTGCAGAGAAGGTCACACTGG |
| | | ACCAAAGGGACTATGAGAAGTATTCAGTGTCCTGCCAGGAGGATGTC |
| | | ACCTGCCCAACTGCCGAGGAGACCCTGCCCATTGAACTGGCGTTGGA |
| | | AGCACGGCAGCAGAATAAATATGAGAACTACAGCACCAGCTTCTTCA |
| | | TCAGGGACATCATCAAACCAGACCCGCCCAAGAACTTGCAGATGAAG |
| | | CCTTTGAAGAACTCACAGGTGGAGGTCAGCTGGGAGTACCCTGACTC |
| | | CTGGAGCACTCCCCATTCCTACTTCTCCCTCAAGTTCTTTGTTCGAATC |
| | | CAGCGCAAGAAAGAAAAGATGAAGGAGACAGAGGAGGGGTGTAACC |
| | | AGAAAGGTGCGTTCCTCGTAGAGAAGACATCTACCGAAGTCCAATGC |
| | | AAAGGCGGGAATGTCTGCGTGCAAGCTCAGGATCGCTATTACAATTC |
| | | CTCATGCAGCAAGTGGGCATGTGTTCCCTGCAGGGTCCGATCCTAA |

Modifications to the sequence orientation and linker length were then tested for impact on IL-12 signaling, as measured through cell-based reporter assays.

The effect of linker length and subdomain orientation on the signaling capacity of scIL 12 was tested. scIL-12 protein constructs were generated with alternative linker compositions, as shown in the above Tables 4-5. The linkers ranged from 4 amino acids to 15 amino acids in length.

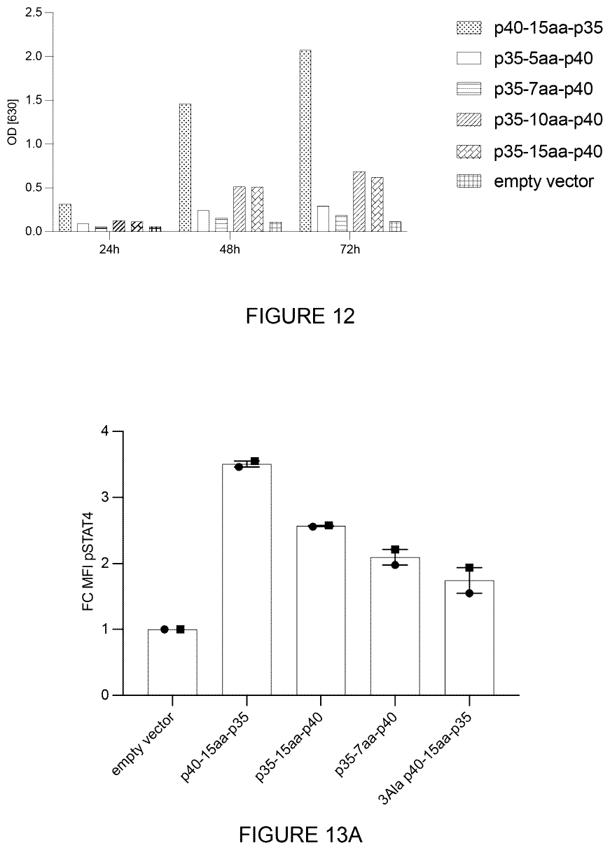

The signaling capacity of these scIL12 variants were observed over time (24, 48, and 72 hours) (FIG. 12). Briefly, 0.18×10⁶ IL-12 reporter cells were seeded in a 24 well plate. After 6 hours, 0.5×10⁶ Jurkat cells expressing scIL-12 variants were added to 0.4 μM cell culture inserts and co-cultured with IL-12 reporter cells. After 24 h, 48 h, and 72 h incubation, the levels of SEAP were determined using QUANTI-Blue™ Solution, a SEAP detection reagent, and by reading the optical density (OD) at 630 nm. Raw data was extracted into Excel files and plotted using Graph Pad.

High levels of signaling were observed from co-cultures in which Jurkat cells expressing and secreting scIL-12 in the p40-p35 orientation were used. Co-cultures in which Jurkat cells expressed and secreted scIL-12 in the p35-p40 orientation showed reduced signaling with a correlation between the length of the linker used and the total signaling observed. Cells expressing scIL-12 p35-7aa-p40 demonstrated the lowest levels of signaling.

The linkers will be further tested for differing structural rigidity and charge compositions in order to observe how these impact on protein conformation and epitope masking.

The inventors hypothesize that when placed in the p40-linker-p35 orientation, the length on the linker will not impair scIL-12 protein function as there is no constraint placed on the protein and the binding interface on the p40 domain remains intact (FIG. 5B-5C). However, when placed in the p35-linker-p40 orientation, the scIL-12 protein is predicted to become constrained, whereby the IL-12 R β1 binding interface is interrupted (FIG. 5D-5E). Thus, interaction with the receptor is predicted to be hindered. Greater levels of constraint are predicted to occur the shorter the linker between the two domains, concomitant with a greater degree of hindrance.

### Example 2: Effect of scIL-12 Variants on Primary T-Cells

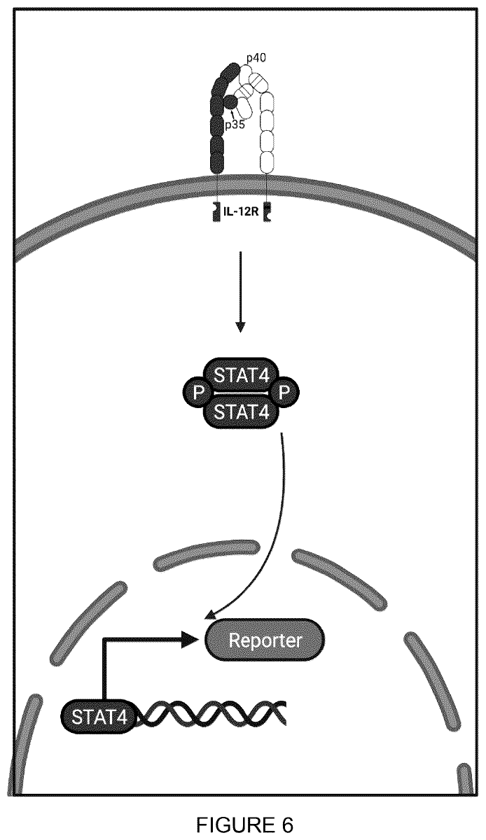

A cell based reported assay was used to demonstrate the signaling capacity of primary T-cell expressing the scIL-12 variants (FIG. 6). It was expected that stimulation through the T-cell receptor (TCR) would enhance IL-12 sensitivity through the upregulation of IL-12 receptor subunits.

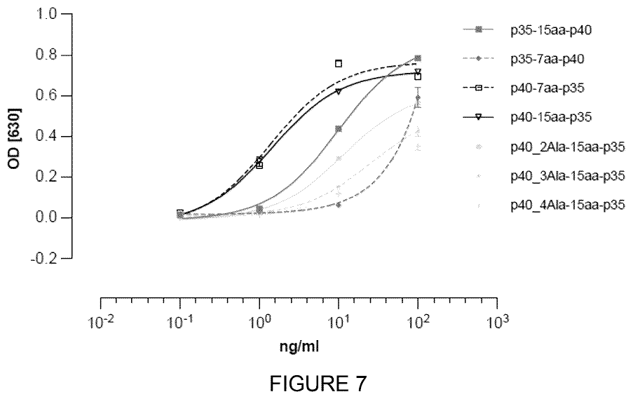

The cell based reported assay was conducted using purified recombinant proteins. 0.5×10⁵ IL12 reporter cells were seeded in a 96-well plate and cultured in a media containing the indicated scIL-12 purified recombinant proteins. Proteins were normalized to the same starting concentration followed by serial dilution. After a 24 hour incubation, the levels of SEAP were determined using QUANTI-Blue™ Solution, a SEAP detection reagent, and by reading the optical density (OD) at 630 nm. Raw data were extracted into Excel files and plotted using GraphPad Prism 9.0. No change in signaling was observed with scIL-12 proteins produced in the p40-p35 orientation (SEQ ID NOs: 1 and 2), despite changes in linker length between the two domains (FIG. 7). When placed in the p35-p40 orientation, scIL-12 p35-15aa-p40 (SEQ ID NO: 3) demonstrated reduced signaling when compared to the p40-15AA-p35 construct (EC50 of 9.1 and 1.4 ng/ml respectively). The scIL-12 p35-p40 orientated construct demonstrated further reduction in its signaling capacity (EC50 2099.3 ng/ml) when limited to a 7 amino acid linker (p35-7AA-p40, or SEQ ID NO: 5).

Prior studies (Glassman et al 2021) demonstrated that mutating residues in the p40 domain of IL-12 that interface with IL-12 RB1 can abrogate binding, leading to decreased IL-12 signaling and lower toxicity in animal models. Three such affinity detuned variants, with threshold affinities low enough to obviate toxicity, were produced and tested in scIL-12 (p40-15AAp35) format. These constructs were SEQ ID NO: 7-9, and included either two, three, or four alanine substitutions. EC50 values of affinity detuned IL-12 were 10, 25, and 27.2 ng/ml for two, three, or four alanine substituted variants respectively.

Stimulation through the T cell receptor (TCR) enhances IL-12 sensitivity through upregulation of IL-12 receptor subunits. To further characterize scIL-12 variants, IFNγ measured by intracellular cytokine stain and supernatant ELISA and STAT4 phosphorylation were quantified in stimulated primary T cells following exposure to scIL-12 variants.

0.18×10⁶ IL-12 reporter cells were seeded in a 24 well plate. After 6 hours, 106 primary T cells expressing scIL-12 variants were added to 0.4 μM cell culture inserts and co-cultured with IL-12 reporter cells. After 24 h, 48 h, and 72 h incubation, the levels of SEAP were determined using QUANTI-Blue™ Solution, a SEAP detection reagent, and by reading the optical density (OD) at 630 nm (FIG. 11). Raw data was extracted into Excel files and plotted using Graph Pad.

The signaling capacity of scIL12 variants was observed over time (24, 48 and 72 hours). High levels of signaling were observed from co-cultures in primary T cells expressing and secreting scIL-12 in the p40-p35 orientation. Co-cultures in which primary T cells expressed and secreted scIL-12 in the p35-p40 orientation showed reduced signaling with a correlation between the length of the linker used and the total signaling observed. Cells expressing scIL-12 p35-7aa-p40 demonstrated the lowest levels of signaling.

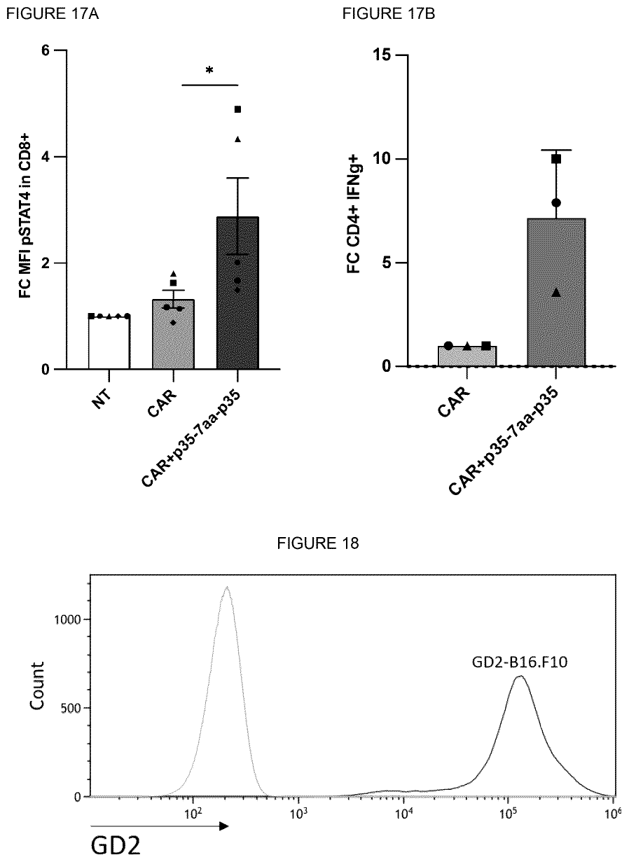

Primary T cells transduced with a CAR expressing vector were exposed to scIL-12 p35-7aa-p40 for 72 h (FIGS. 17A-17B). Cells were stained through their cytoplasms for the marker of interest and analyzed on a MacsQuant Analyzer (Miltenyi) flow cytometer. At least 1×10⁴ events were collected for each sample. FlowJo software (FlowJo, LLC) was used for data analysis, with serial gating. High levels of pSTAT4 signaling were observed in CD8+ primary CAR-T cells following exposure to scIL-12 p35-7aa-p40 (FIG. 17A). Similarly, high levels of IFNγ signaling were observed in CD4+ primary CAR-T cells following exposure to scIL-12 p35-7aa-p40, suggesting a differentiation of CD4 cells towards Th1 (FIG. 17B).

As IL-12 is known to support Th1 polarization, naïve primary CD4+ T cells activated with αCD3 and αCD28 antibodies will also be quantified for Th1 induction in response to scIL-12 variants.

### Example 3: Detection of Modified scIL-12 Variants Secreted from Cells

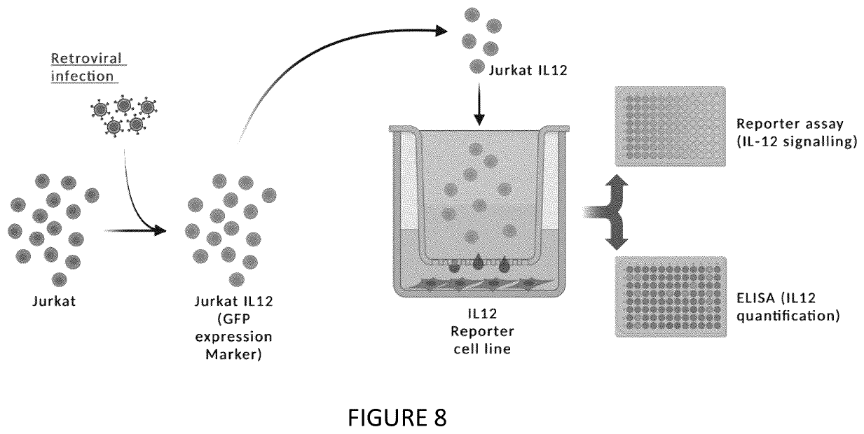

Jurkat cells were transduced using a retroviral vector encoding the sc1L-12 variants along with GFP as a marker for transduction in a single bicistronic cassette. Jurkat cells expressing scIL-12 were then used in transwell assays as depicted in FIG. 8 to measure the signaling capacity of scIL-12 secreted by a T-cell line. The quantity of scIL-12 secreted from each cell line was further evaluated by ELISA.

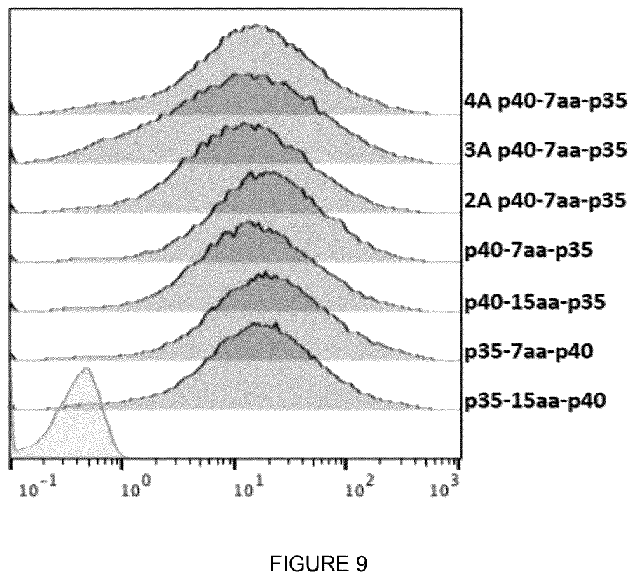

Constructs also included a GFP sequence, such that GFP expression can be used as a marker for transduction and expression in cells. Transduction of cells expressing scIL-12 variants demonstrated comparable levels of GFP expression (92-96% GFP expressing cells), suggesting that all the constructs where highly transduced within the target cells (FIG. 9).

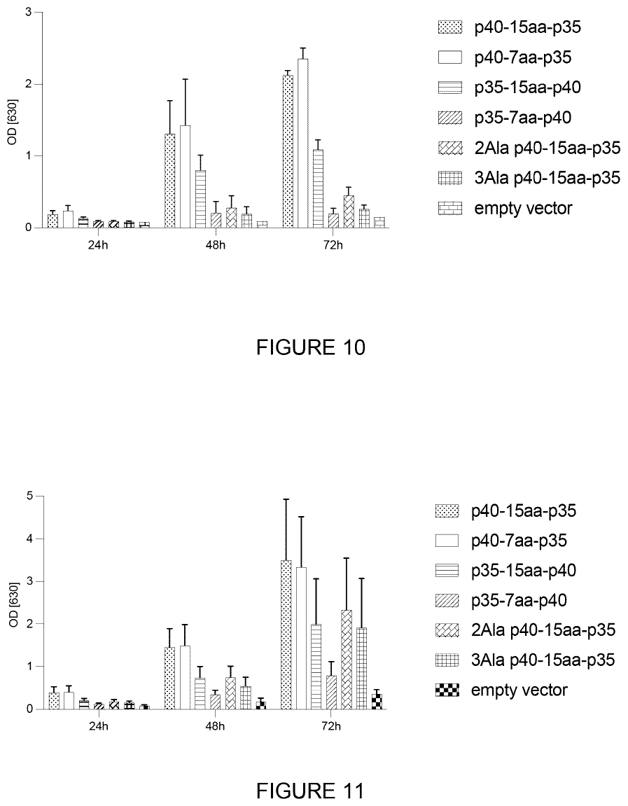

0.18×10⁶ IL-12 reporter cells were seeded in a 24 well plate. After 6 hours, 0.5×10⁶ Jurkat expressing scIL-12 variants were added to 0.4 μM cell culture inserts and co-cultured with IL-12 reporter cells. After 24 h, 48 h, and 72 h incubation, the levels of SEAP were determined using QUANTI-Blue™ Solution, a SEAP detection reagent, and by reading the optical density (OD) at 630 nm (FIG. 10). Raw data was extracted into Excel files and plotted using Graph Pad.

High levels of signaling were observed from co-cultures in which Jurkat cells expressing and secreting scIL-12 in the p40-p35 orientation were used. The strength of signaling observed in this orientation was independent of the linker length used. Co-cultures in which Jurkat cells expressed and secreted scIL-12 in the p35-p40 orientation showed reduced signaling with a correlation between the length of the linker used and the total signaling observed. Cells expressing scIL-12 p35-7AA-p40 (SEQ ID NO: 5) demonstrated the lowest levels of signaling, comparable with affinity detuned mutations with preferable toxicity profiles.

### Example 4: Effect of scIL-12 Variants on NK Cells

Wild type IL-12 signaling is known to result in the induction of IFNγ in both T-cells and NK cells, leading to toxicity associated with heightened production. One goal of the mutated IL-12 protein constructs of the present disclosure is to reduce and/or eliminate toxicity. Given that, IL-12 mutants were screened for their ability to reduce induction of IFNγ and effect phosphorylation of STAT4 (pSTAT4).

In order to characterize IFNγ levels in purified NK cells, the latter was co-cultured with primary T-cells expressing and secreting wild type or scIL-12 variants.

0.5×10⁶ primary NK cells were isolated and cultured using the CellXVivo Human NK Cell Expansion Kit (Bio-Techne). Following culture, cells were seeded in a 24-well plate. 106 primary T cells expressing scIL-12 variants were added to 0.4 μM cell culture inserts and co-cultured with NK cells for 3 days. NK cells were then stained for CD56 and subsequently fixed and permeabilized using the Phosflow Perm Buffer III (BDBiosciences) to allow for pSTAT4 (BDBiosciences) and IFNγ (Biolegend) staining. The levels of pSTAT4 were determined using the MACSQuant® Analyzer 10 Flow Cytometer and analyzed using the FlowJo (v10) software. Raw data from each population was extracted into Excel files and plotted using Graph Pad Prism 9.0. Supernatant from the co-culture was used for IFNγ ELISA detection using the ELISAMAX™ Deluxe Set Human IFN-γ kit from Biolegend.

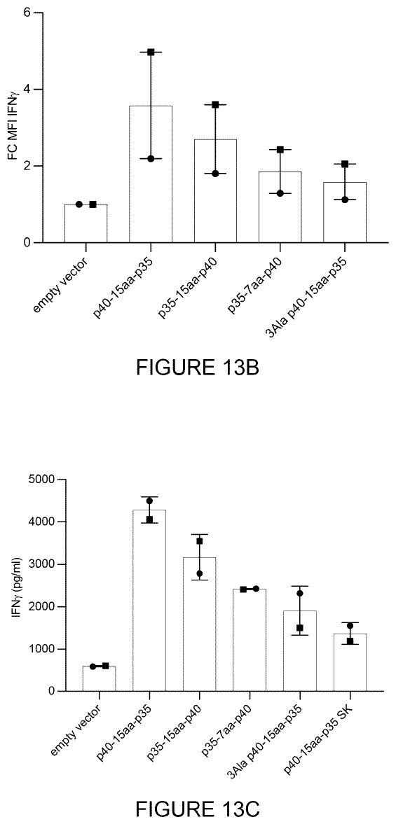

After 96 hours of coculturing NK cells with primary T cells expressing and secreting scIL-12 variants, pSTAT4 and IFN-γ were measured by ELISA and intracellular staining (FIGS. 13A-13C).

Given that IL-12 is known to support Th1 polarization, naive primary CD4+ T-cells are predicted to be activated with αCD3 and αCD28 antibodies. This will be quantified by monitoring Th1 induction in response to scIL-12 variants.

### Example 5: Augmentation of CAR T-Cell Efficacy Using scIL-12 in an In-Vivo Setting

Constructs will be generated for expressing GD2 CAR, GD2 CAR/IL-12 and GD2 CAR/scIL-12. The GD2 CAR represents a murine version of the IL-12 constructs. The GD2 CAR construct is comprised as shown in Tables 9-10, and as follows:
GD2 CAR:
IgG HV region signal peptide (SEQ ID NO: 48)—VH Sequence (SEQ ID NO: 49)—VL Sequence (SEQ ID NO: 50)—CD8 Hinge Sequence (SEQ ID NO: 51)—CD28 Transmembrane Sequence (SEQ ID NO: 52)—CD28 Co-Stimulatory Domain (SEQ ID NO: 53)—CD35 (SEQ ID NO: 54)

TABLE 10
Amino Acid Sequences of GD2 CAR Domains

| SEQ ID | | |
|---|---|---|
| NO: | Description | Sequence |
| 51 | IgG HV | MGWSCIILFLVATATGVHS |
| | region signal | |
| | peptide | |
| 52 | VH | QVQLKESGPVLVAPSQTLSITCTVSGFSLASYNTHWVRQPPGKGLEWLG |
| | | VIWAGGSTNYNSALMSRLSISKDNSKSQVFLQMNSLQTDDTAMYYCAK |
| | | RSDDYSWFAYWGQGTLVTVSA |
| 53 | VL | ENVLTQSPAIMSASPGEKVTMTCRASSSVSSSYLHWYQQKSGASPKVWI |
| | | YSTSNLASGVPGRFSGSGSGTSYSLTISSVEAEDAATYYCQQYSGYPITFG |
| | | AGTKVEVK |
| 54 | CD8 Hinge | AKPTTTPAPRPPTPAPTIASQPLSLRPEACRPAAGGAVHTRGLDFACD |
| 55 | CD28 | FWVLVVVGGVLACYSLLVTVAFIIFWV |
| | Transmembrane | |
| 56 | CD28 Co- | RSKRSRLLHSDYMNMTPRRPGPTRKHYQPYAPPRDFAAYRS |
| | Stimulatory | |
| 57 | CD3ζ | RVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLST |
| | | ATKDTYDALHMQALPPR |

TABLE 9
DNA Sequences of GD2 CAR Domains

| SEQ ID | | |
|---|---|---|
| NO: | Description | Sequence |
| 58 | IgG HV | ATGGGCTGGAGCTGCATCATCCTGTTCCTGGTGGCCACCGCCACCGGC |
| | region signal | GTGCACAGC |
| | peptide | |
| 59 | VH | CAGGTTCAGCTTAAGGAAAGCGGGCCTGTCCTTGTAGCTCCCTCCCAG |
| | | ACACTTTCAATCACATGCACCGTATCAGGGTTTTCCCTCGCTAGCTAC |
| | | AATACCCATTGGGTACGGCAACCACCCGGCAAAGGACTTGAGTGGTT |
| | | GGGCGTTATTTGGGCTGGCGGTTCTACAAATTACAATAGTGCACTGAT |
| | | GTCCCGCCTCAGCATAAGTAAAGACAACTCAAAATCTCAGGTATTTTT |
| | | GCAGATGAACAGTCTTCAGACAGATGACACCGCCATGTACTATTGCG |
| | | CAAAACGATCAGACGATTACTCTTGGTTCGCTTACTGGGGGCAGGGC |
| | | ACATTGGTTACCGTCTCTGCT |
| 60 | VL | GAAAATGTCTTGACTCAATCCCCTGCCATAATGAGTGCAAGTCCAGG |
| | | AGAAAAGGTCACTATGACCTGCCGGGCTTCAAGTTCAGTTAGTTCAA |
| | | GTTATTTGCACTGGTATCAACAAAAAAGTGGGGCCTCACCCAAGGTA |
| | | TGGATATACAGCACATCCAACCTTGCTTCTGGTGTGCCAGGTAGGTTT |
| | | TCTGGTTCCGGTAGCGGGACCTCTTACTCCTTGACCATTAGCTCCGTA |
| | | GAGGCAGAGGACGCTGCTACCTACTACTGTCAACAGTACTCTGGTTAT |
| | | CCCATAACATTTGGAGCCGGTACTAAAGTTGAAGTCAAG |
| 61 | CD8 Hinge | gcgaagcccaccacgacgccagcgccgcgaccaccaacaccggcgcccaccatcgcgtcgcagcc |
| | | cctgtccctgcgcccagaggcgtgccggccagcggcggggggcgcagtgcacacgagggggctgg |
| | | acttcgcctgtgat |
| 62 | CD28 | ttttgggtgctggtggtggttggtggagtcctggcttgctatagcttgctagtaacagtggcctt |
| | Transmembrane | tattattttctgggtg |
| 63 | CD28 Co- | aggagtaagaggagcaggctcctgcacagtgactacatgaacatgactccccgccgccccgggcc |
| | Stimulatory | cacccgcaagcattaccagccctatgccccaccacgcgacttcgcagcctatcgctcc |
| 64 | CD3ζ | agagtgaagttcagcaggagcgcagacgcccccgcgtaccagcagggccagaaccagctctataa |
| | | cgagctcaatctaggacgaagagaggagtacgatgttttggacaagaggcgtggccgggaccctg |
| | | agatggggggaaagccgagaaggaagaaccctcaggaaggcctgtacaatgaactgcagaaagat |
| | | aagatggcggaggcctacagtgagattgggatgaaaggcgagcgccggaggggcaaggggcacga |
| | | tggcctttaccagggcctcagtacagccaccaaggacacctacgacgcccttcacatgcaggccc |
| | | tgccccctcgc |

The in vivo efficacy of the constructs will then be quantified in a B16.F10 melanoma immunocompetent mouse model. C57BL/6 mice will be injected subcutaneously with 1×10⁵ B16.F10 melanoma cells, and transduced with a gamma-retroviral vector encoding the enzymes GD2 and GD3 synthases to synthesize ganglioside GD2, before being allowed to engram for 6 days prior to 5 Gy total body irradiation. On day 7, non-transduced and transduced CAR cohorts of C57Bl/6 splenocytes will be intravenously injected into mice. Tumor growth and survival will be monitored for a determined number of days post engraftment. Serum cytokine levels will be detected from bloods collected by tail vein bleeds.

It is expected that prolonged survival and enhanced tumor clearance will only be demonstrated in mice receiving GD2 CAR/scIL-12 cells. Mice receiving non-transduced and GD2 CAR cells are expected to fail to clear tumors, and will need to be culled due to tumor burden. Clearance is expected to be observed in GD2 CAR-IL-12 treated mice, but they will also need to be culled due to ill health and decreased weight.

### Example 6: Enhanced Safety of IL-12 Demonstrated in an In-Vivo Setting

Constructs will be generated expressing GD2 CAR, GD2 CAR/IL-12 and GD2 CAR/scIL-12. The in vivo efficacy of the constructs will then be quantified in a B16.F10 melanoma immunocompetent mouse model. C57BL/6 mice will be injected subcutaneously with 1×10⁵ B16.F10 melanoma cells, and transduced with a gamma-retroviral vector encoding the enzymes GD2 and GD3 synthases to synthesize ganglioside GD2. They then will be allowed to engram for 6 days prior to 5 Gy total body irradiation. On day 7, non-transduced and transduced CAR cohorts of C57Bl/6 splenocytes will be intravenously injected into mice. Tumor growth and survival will be monitored for a determined number of days post engraftment.

It is predicted that the GD2 CAR/scIL-12 cohorts will show enhanced tumor clearance, which will be evident through the decreased tumor volume during the study period. It is also predicted that the GD2 CAR/scIL-12 cohorts will have an enhanced survival rate compared to mice receiving non-transduced cells and GD2 CAR cells and GD2 CAR/IL-12 cells.

### Example 7: Impact of scIL-12 Variations on STAT4 Phosphorylation and IFN-γ

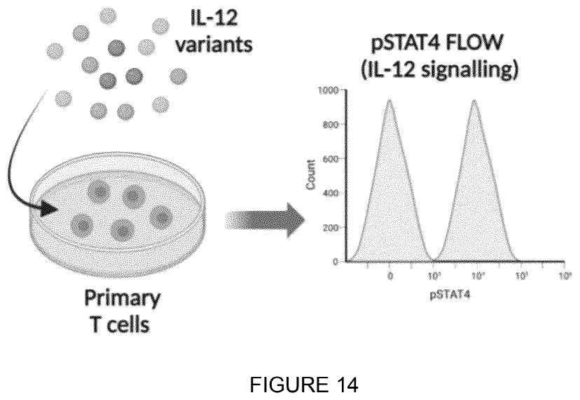

Purified scIL-12 purified recombinant protein were purified and added to activated T cells to determine the impact of the scIL-12 variants on STAT4 phosphorylation and IFN-γ production, a schematic of which is depicted in FIG. 14.

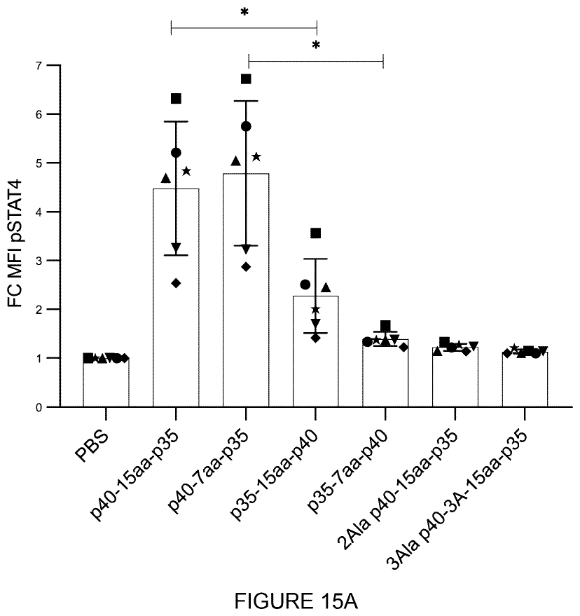

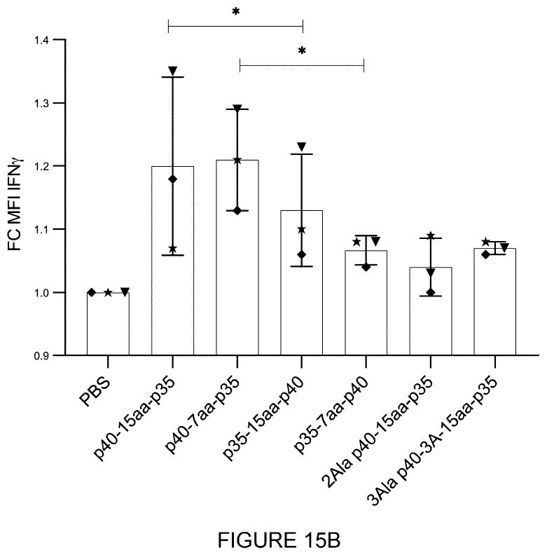

In order to test for pSTAT4 levels, 0.25×10⁶ activated primary T cells were seeded in a 96-well plate and cultured in a media containing 100 ng/μL of the indicated scIL-12 purified recombinant proteins (FIG. 15A). Each protein was normalized to the same concentration and added to T cells that were previously activated with anti-CD3/CD28 in the presence of IL-2. After a 30-minute incubation, the cells were stained for CD8 and subsequently fixed and permeabilized using the Phosflow Perm Buffer III (BD Biosciences) to allow for pSTAT4 (BD Biosciences) staining. The levels of pSTAT4 were determined using the MACSQuant® Analyzer 10 Flow Cytometer and analyzed using the FlowJo (v10) software. Raw data from each population was extracted into Excel files and plotted using GraphPad Prism 9.0.

No change in signaling was observed with scIL-12 proteins produced in the p40-p35 orientation despite changes in linker length between the two domains (FIG. 15A). When placed in the p35-p40 orientation, however, scIL-12 p35-15aa-p40 demonstrated reduced signaling when compared to the p40-15AA-p35 construct. ScIL-12 p35-7AA-p40 demonstrated a further reduction in signaling capacity.

In order to test for IFN-γ levels, 0.25×10⁶ activated primary T cells were seeded in a 96-well plate and cultured in a media containing of the indicated scIL-12 purified recombinant proteins (FIG. 15B). Each protein was normalized to the same concentration and added to T cells that were previously activated with anti-CD3/CD28 in the presence of IL-2. After a 24 hour incubation, the cells were stained for CD8 and subsequently fixed and permeabilized using the Phosflow Perm Buffer III (BD Biosciences) to allow for IFN-γ (Biolegend) staining. The levels of IFN-γ were determined using the MACSQuant® Analyzer 10 Flow Cytometer and analyzed using the FlowJo (v10) software. Raw data from each population was extracted into Excel files and plotted using GraphPad Prism 9.0.

No change in signaling was observed with scIL-12 proteins produced in the p40-p35 orientation despite changes in linker length between the two domains (FIG. 15B). When placed in the p35-p40 orientation, however, scIL-12 p35-15aa-p40 demonstrated reduced signaling when compared to the p40-15AA-p35 construct. ScIL-12 p35-7AA-p40 demonstrated a further reduction in signaling capacity.

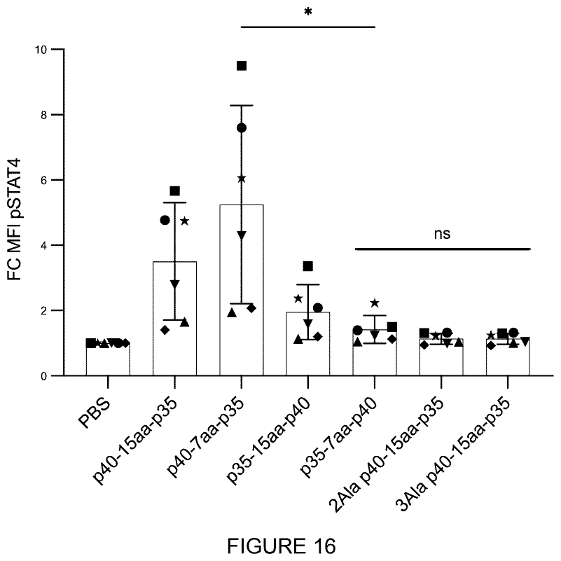

Next, the pSTAT4 levels were assessed in the presence of NK cells. 0.25×10⁶ activated primary NK cells were isolated and cultured using the CellXVivo Human NK Cell Expansion Kit (Bio-Techne). These cells were then seeded in a 96-well plate and cultured in a media containing 20 ng/μL of the indicated scIL-12 purified recombinant proteins (FIG. 16). Each protein was normalized to the same concentration and added to NK cells that were previously isolated from healthy donors. After a 30-minute incubation, the cells were stained for CD56 and subsequently fixed and permeabilized using the Phosflow Perm Buffer III (BD Biosciences) to allow for pSTAT4 (BD Biosciences) staining. The levels of pSTAT4 were determined using the MACSQuant® Analyzer 10 Flow Cytometer and analyzed using the FlowJo (v10) software. Raw data from each population was extracted into Excel files and plotted using GraphPad Prism 9.0.

No change in signaling was observed with scIL-12 proteins produced in the p40-p35 orientation despite changes in linker length between the two domains (FIG. 16). When placed in the p35-p40 orientation, however, scIL-12 p35-15aa-p40 demonstrated reduced signaling when compared to the p40-15AA-p35 construct. ScIL-12 p35-7AA-p40 demonstrated a further reduction in signaling capacity.

### Example 8: Augmentation of CAR T Cell Efficacy Using scIL-12 in an In-Vivo Setting

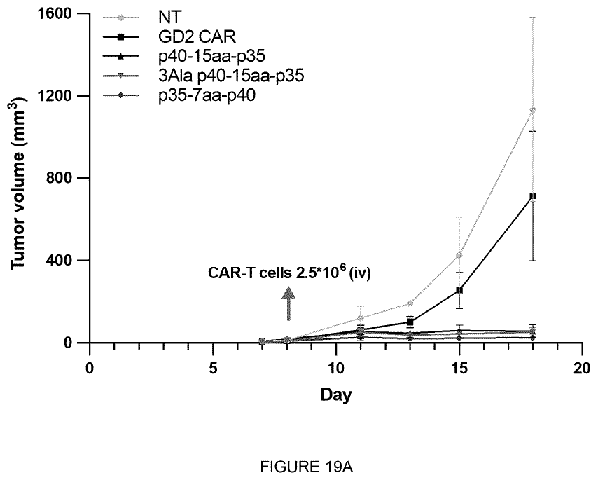

B16.F10 melanoma cell lines were engineered to express the enzymes GD2 and GD3 synthases, which function to synthesize ganglioside GD2. The over-expression of ganglioside GD2 in B16.F10 melanoma cell line was confirmed by flow cytometry (FIG. 18).

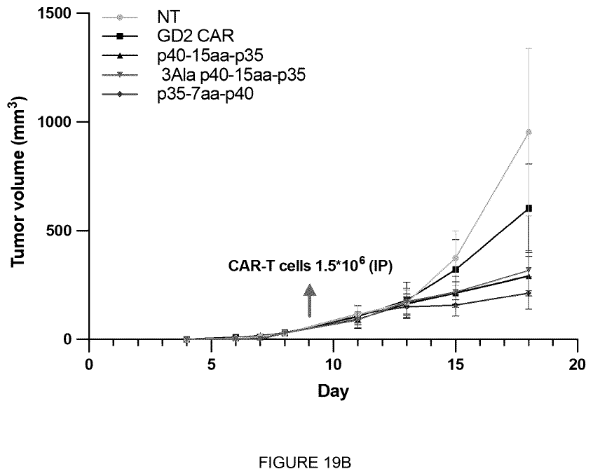

C57BL/6 mice were injected subcutaneously with 1×10⁵ of these engineered B16.F10 melanoma cells and allowed to engraft for 8 days. On day 8, each mouse was subject to CAR injection. Non-transduced and transduced CAR cohorts of C57Bl/6 splenocytes were intravenously (2.5×10⁶ CAR cells) or intraperitoneally (1.5×10⁶ CAR cells) injected into mice. Tumours were measured 3 times a week and tumour volumes were estimated using the formula 0.5 (LxW2) by measuring the tumour in two dimensions using electronic calipers for the duration of the study (FIGS. 19A-19B). Prolonged survival and enhanced tumour clearance was only demonstrated in mice receiving GD2 CAR/scIL-12 cells. Mice receiving non-transduced or GD2 CAR cells by intravenous (FIG. 19A) or intraperitoneal (FIG. 19B) administration failed to clear their tumours, whereas mice receiving GD2 CAR/scIL-12 cells in each group showed a significantly reduced tumour burden.

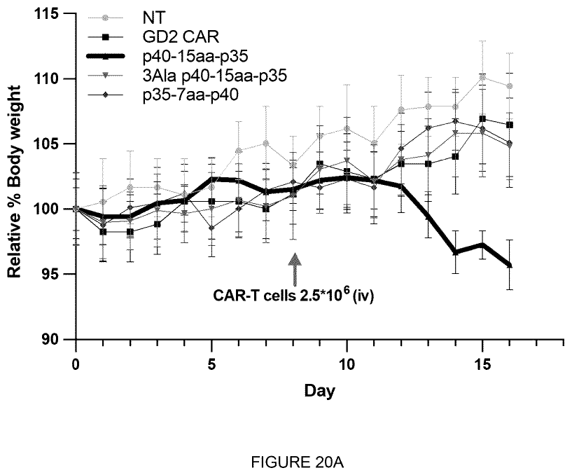

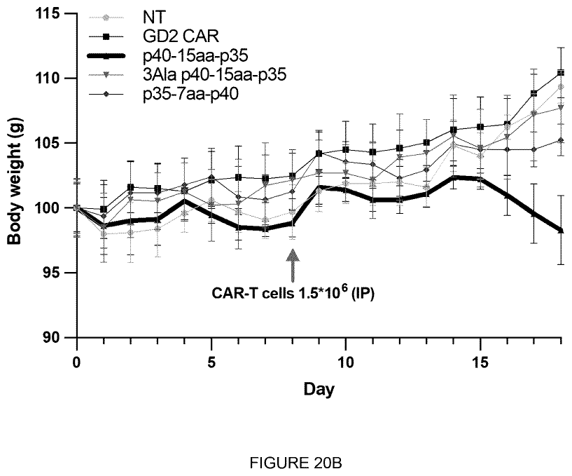

Following CAR injection, mice were weighed and monitored daily for any sign of distress (FIGS. 20A-20B). Mice receiving CAR/scIL-12 p40-15aa-p35 cells showed reduced body weight, whereas mice receiving CAR with modified scIL-12 show no sign of body weight loss.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through second aspects is applicable to all aspects and embodiments identified herein. Moreover, any of the features of an embodiment of the first through third aspects is independently combinable, partly or wholly with other embodiments described herein in any way, e.g., one, two, or three or more embodiments may be combinable in whole or in part. Further, any of the features of an embodiment of the first through third aspects may be made optional to other aspects or embodiments.

1. A modified IL-12 protein construct comprising a p35 subdomain, a linker, and a p40 subdomain.

2. The modified IL-12 protein construct of claim 1, wherein the linker is from 4 to 15 amino acids in length.

3. The modified IL-12 protein construct of claim 1, wherein the p35 subdomain is on the C terminus, and the p40 subdomain is on the N terminus.

4. The modified IL-12 protein construct of claim 1, wherein the p35 subdomain is on the N terminus, and the p40 subdomain is on the C terminus.

5. The modified IL-12 protein construct of claim 1, wherein the linker comprises a sequence with at least 80% identity to any one of the sequences of SEQ ID NOs: 23-26.

6. The modified IL-12 protein construct of claim 1, wherein the p40 subdomain comprises a sequence with at least 80% identity to any one of the sequences of SEQ ID NOs: 19 or 20.

7. The modified IL-12 protein construct of claim 6, wherein the p40 subdomain further comprises one, two, three, or four mutations.

8. The modified IL-12 protein construct of claim 7, wherein the mutations comprise mutations of E81A, F82A, K106A, or K219A.

9. The modified IL-12 protein construct of claim 1, wherein the p35 subdomain comprises a sequence with at least 80% identity to any one of the sequences of SEQ ID NOs: 21 or 22.

10. The modified IL-12 protein construct of claim 1, wherein the construct comprises a sequence with at least 80% identity to any one of the sequences of SEQ ID NOs: 1-9.

11-15. (canceled)

16. A cell containing and/or secreting a modified IL-12 protein construct comprising a p35 subdomain, a linker, and a p40 subdomain.

17. The cell of claim 16, wherein the cell is a lymphocyte and/or leukocyte.

18. The cell of claim 17, wherein the cell is a T lymphocyte or a cytotoxic T lymphocyte.

19. The cell of claim 16, wherein the modified IL-12 protein has a reduced affinity for IL-12 receptors compared to wild type IL-12.

20. The cell of claim 16, wherein the modified IL-12 protein has a reduced stability and/or half-life compared to wild type IL-12.

21. The cell of claim 16, wherein the modified IL-12 protein has a reduced expression compared to wild type IL-12.

22. The cell of claim 21, wherein the reduced expression comprises reduced concentration of IL-12 in a cell, a tissue, an organ, a blood sample, a plasma sample, a system, and/or a subject.

23. The cell of claim 16, wherein the cell leads to decreased level of IFNγ production by effector cells when compared to a cell expressing wild type IL-12.

24-26. (canceled)

27. A method of treating cancer in a subject, the method comprising administering to the subject a modified IL-12 protein construct comprising a p35 subdomain, a linker, and a p40 subdomain.

28-36. (canceled)

37. The method of claim 27, wherein the cancer is pancreatic cancer, prostate cancer, breast cancer, ovarian cancer, colorectal cancer, lung cancer, renal cell carcinoma, bladder cancer, medulloblastoma or neuroblastoma.

38-71. (canceled)
